(19) United States
(12) Patent Application Publication
(10) Pub. No.: US 2018/0133252 A9
(43) Pub. Date: May 17, 2018

(54) CHIMERIC RECEPTORS AND USES THEREOF IN IMMUNE THERAPY

(71) Applicant: Unum Therapeutics Inc., Cambridge, MA (US)

(72) Inventors: Charles Wilson, Cambridge, MA (US); Kathleen McGinness, Cambridge, MA (US)

(73) Assignee: Unum Therapeutics Inc., Cambridge, MA (US)

(21) Appl. No.: 15/509,133

(22) PCT Filed: Sep. 9, 2015
(86) PCT No.: PCT/US2015/049126

§ 371 Date: Mar. 6, 2017

Related U.S. Application Data
(60) Provisional application No. 62/047,916, filed on Sep. 9, 2014.

(51) Int. Cl. A61K 35/17; C07K 14/735; A61K 39/395; C12N 15/86; C12N 5/0783; C07K 16/32; C07K 16/30

(52) U.S. Cl. CPC A61K 35/17; C07K 14/70535; A61K 39/395; C12N 15/86; C12N 5/0636; C07K 16/32; A61K 2039/505; C07K 2319/03; C07K 2319/02; C07K 2319/70; C12N 2740/10043; C12N 2510/00; C07K 2317/732; C07K 16/3084

(57) ABSTRACT

Disclosed herein are chimeric receptors comprising an extracellular domain with affinity and specific for the Fc portion of an immunoglobulin molecule (Ig), an Fc-binding domain; a transmembrane domain; at least one co-stimulatory signaling domain; and a cytoplasmic signaling domain comprising an immunoreceptor tyrosine-based activation motif (ITAM). Also provided herein are nucleic acids encoding such chimeric receptors and immune cells expressing the chimeric receptors. Such immune cells can be used to enhance antibody-dependent cell-mediated cytotoxicity and/or to enhance antibody-based immunotherapy, such as cancer immunotherapy.

### CROSS REFERENCE TO RELATED APPLICATIONS

This application claims the benefit of U.S. Provisional Application No. 62/047,916, filed Sep. 9, 2014, under 35 U.S.C. §119, the entire content of which is herein incorporated by reference.

### BACKGROUND OF DISCLOSURE

Cancer immunotherapy, including cell-based therapy, antibody therapy and cytokine therapy, is used to provoke immune responses attacking tumor cells while sparing normal tissues. It is a promising option for treating various types of cancer because of its potential to evade genetic and cellular mechanisms of drug resistance, and to target tumor cells while sparing normal tissues. T-lymphocytes can exert major anti-tumor effects as demonstrated by results of allogeneic hematopoietic stem cell transplantation (HSCT) for hematologic malignancies, where T-cell-mediated graft-versus-host disease (GvHD) is inversely associated with disease recurrence, and immunosuppression withdrawal or infusion of donor lymphocytes can contain relapse. Weiden et al., NEnglJ Med. 1979;300(19):1068-1073; Porter et al., NEnglJ Med. 1994;330(2):100-106; Kolb et al., Blood. 1995;86(5):2041-2050; Slavin et al., Blood. 1996;87(6):2195-2204; and Appelbaum, Nature. 2001;411(6835):385-389.

Cell-based therapy may involve cytotoxic T cells having reactivity skewed toward cancer cells. Eshhar et al., Proc. Natl. Acad. Sci. U. S. A.; 1993;90(2):720-724; Geiger et al., J Immunol. 1999;162(10):5931-5939; Brentjens et al., Nat. Med. 2003;9(3):279-286; Cooper et al., Blood. 2003;101(4):1637-1644; and Imai et al., Leukemia. 2004;18:676-684. One approach is to express a chimeric antigen receptor having an antigen-binding domain fused to one or more T cell activation signaling domains. Binding of a cancer antigen via the antigen-binding domain results in T cell activation and triggers cytotoxicity. Recent results of clinical trials with infusions of chimeric receptor-expressing autologous T lymphocytes provided compelling evidence of their clinical potential. Pule et al., Nat. Med. 2008;14(11):1264-1270; Porter et al., N Engl J Med; 2011; 25;365(8):725-733; Brentjens et al., Blood. 2011;118(18):4817-4828; Till et al., Blood. 2012;119(17):3940-3950; Kochenderfer et al., Blood. 2012;119(12):2709-2720; and Brentjens et al., Sci Transl Med. 2013;5(177):177ra138.

Antibody-based immunotherapies, such as monoclonal antibodies, antibody-fusion proteins, and antibody drug conjugates (ADCs) are used to treat a wide variety of diseases, including many types of cancer. Such therapies may depend on recognition of cell surface molecules that are differentially expressed on cells for which elimination is desired (e.g., target cells such as cancer cells) relative to normal cells (e.g., non-cancer cells). Binding of an antibody-based immunotherapy to a cancer cell can lead to cancer cell death via various mechanisms, e.g., antibody-dependent cell-mediated cytotoxicity (ADCC), complement-dependent cytotoxicity (CDC), or direct cytotoxic activity of the payload from an antibody-drug conjugate (ADC).

### SUMMARY OF DISCLOSURE

The present disclosure is based on the design of chimeric receptors comprising an extracellular domain with affinity and specificity for the Fc portion of an immunoglobulin (Ig), such as an IgG antibody, a transmembrane domain, at least one co-stimulatory signaling domain, and a cytoplasmic signaling domain that comprises an immunoreceptor tyrosine-based activation motif (ITAM). Immune cells expressing such a chimeric receptor construct would enhance efficacy of immune therapy such as antibody-based immunotherapies via, e.g., enhancing ADCC activity.

Accordingly, one aspect of the present disclosure features a chimeric receptor that comprises (a) an extracellular domain that binds to the Fc portion of an immunoglobulin (an Fc-binding domain), e.g., binds the Fc portion of an IgG; (b) a transmembrane domain; (c) at least one co-stimulatory signaling domain; and (d) a cytoplasmic signaling domain that comprises an ITAM. Either the at least one co-stimulatory signaling domain or the cytoplasmic signaling domain that comprises an ITAM can be localized at the C-terminus of a chimeric receptor construct as described herein. In some embodiments, the ITAM-containing cytoplasmic signaling domain is located at the C-terminus of a chimeric receptor construct. In some embodiments, (a) is an extracellular ligand-binding domain of CD16 (e.g., CD16A or CD16B) and (d) does not comprise an ITAM of an Fc receptor. In some embodiments, (d) is a cytoplasmic signaling domain of CD3ξ or FcζR1γ. Any of the chimeric receptors described herein may further comprise (e) a hinge domain, which can be located at the C-terminus of (a) and the N-terminus of (b).

In some embodiments, (a) of the chimeric receptor construct described herein is an extracellular ligand-binding domain of an Fc receptor such as Fc-gamma receptor, an Fc-alpha receptor, or an Fc-epsilon receptor. For example, (a) can be an extracellular ligand-binding domain of CD16 (e.g., CD16A or CD16B), CD32 (e.g., CD32A, or CD32B), or CD64 (e.g., CD64A, CD64B, or CD64C). In some examples, (a) is not the extracellular ligand-binding domain of CD16. In other embodiments, (a) is an extracellular ligand-binding domain of CD32 (e.g., CD32A, or CD32B).

In other embodiments, (a) is of a non-Fc receptor naturally-occurring protein capable of binding to the Fc portion of an Ig molecule, such as an IgG molecule. For example, (a) may be all or part of protein A or protein G. Alternatively, (a) may be an antibody fragment that binds the Fc portion of an IgG molecule, including, but not limited to a single-chain variable fragment (scFv), or a domain antibody, a nanobody.

In yet other embodiments, (a) is a designed (e.g., non-naturally occurring) peptide capable of binding to the Fc portion of an IgG molecule, including a Kunitz domain peptide, a small modular immunopharmaceutical (SMIP), an adnectin, an avimer, an affibody, a DARPin, or an anticalin.

Alternatively or in addition, the transmembrane domain of the chimeric receptor of (b) can be of a single-pass membrane protein, including, but not limited to, CD8α, CD8β, 4-1BB, CD28, CD34, CD4, FcδRIγ, CD16 (e.g., CD16A or CD16B), OX40, CD3ζ, CD3ϵ, CD3γ, CD3δ, TCRα, CD32 (e.g., CD32A or CD32B), CD64 (e.g., CD64A, CD64B, or CD64C), VEGFR2, FAS, and FGFR2B. In some examples, the membrane protein is not CD8α. The transmembrane domain may also be a non-naturally occurring hydrophobic protein segment.

In any of the chimeric receptor constructs described herein, the at least one co-stimulatory signaling domain of the chimeric receptor described herein may be of a co-stimulatory molecule such as 4-1BB (also known as CD137), CD28, CD28_{LL}_{_{→}}_{GG} variant, OX40, ICOS, CD27, GITR, HVEM, TIM1, LFA1, or CD2. In some examples, the at least one co-stimulatory signaling domain is not from 4-1BB. In some examples, the chimeric receptor comprises two co-stimulatory signaling domains, e.g., CD28 and 4-1BB, or CD28_{LL}_{_{→}}_{GG} variant and 4-1BB.

In any of the chimeric receptors described herein, the hinge domain can be of a protein such as CD8α, or IgG. For example, the hinge domain can be a fragment of the transmembrane or hinge domain of CD8α. In some examples, the hinge domain is not the hinge domain of CD8α. In some examples, the hinge domain is a non-naturally occurring peptide, such as an polypeptide consisting of hydrophilic residues of varying length (XTEN) or a (Gly₄Ser)ₙ polypeptide, in which n is an integer of 3-12, inclusive.

In some embodiments, any of the chimeric receptors described herein may further comprise a signal peptide at its N-terminus, e.g., the signal peptide of CD8α, which may comprise the amino acid sequence of SEQ ID NO:61.

Examples of the chimeric receptors described herein may comprise components (a)-(e) as shown in Table 3, Table 4, and Table 5. In some examples, the chimeric receptor comprises the amino acid sequence selected from SEQ ID NOs:2-30 and 32-56, or a fragment thereof which excludes the signal peptide of a reference sequence.

In specific embodiments, the chimeric receptors described herein may comprise an extracellular ligand-binding domain of F158 FCGR3A (F158 CD16A) or the V158 FCGR3A variant (V158 CD16A). Such an extracellular ligand-binding domain may comprise the amino acid sequence of SEQ ID NO:70 and SEQ ID NO:57, respectively.

In other specific embodiments, the chimeric receptor described herein may comprise a hinge and transmembrane domain of CD8α, which may comprise the amino acid sequence of SEQ ID NO:58.

Alternatively or in addition, the chimeric receptor described herein may comprise a co-stimulatory signaling domain of 4-1BB, which may comprise the amino acid sequence of SEQ ID NO:59.

In yet other specific embodiments, the chimeric receptor described herein may comprise a cytoplasmic signaling domain of CD3ζ, which may comprise the amino acid sequence of SEQ ID NO: 60.

In some examples, the chimeric receptor described here is not a receptor that comprises a signal peptide of CD8α, an extracellular domain of F158 CD16A or V158 CD16A, a hinge and transmembrane domain of CD8α, a co-stimulatory signaling domain of 4-1BB, and a cytoplasmic signaling domain of CD3ζ. In particular examples, the chimeric receptor described herein does not comprise either the amino acid sequence of SEQ ID NO:1 or SEQ ID NO:31.

In another aspect, the present disclosure features a nucleic acid (e.g., a DNA molecule or an RNA molecule) comprising a nucleotide sequence encoding any of the chimeric receptors described herein; vectors (e.g., expression vectors) comprising the nucleic acid; and host cells (e.g., immune cells such as natural killer cells, macrophages, neutrophils, eosinophils, and T cells). In some embodiments, the vector is a viral vector, e.g., a lentiviral vector or a retroviral vector. In some embodiments, the vector is a transposon or contains a transposon.

In some embodiments, the host cell that expresses any of the chimeric receptors described herein is a T lymphocyte or an NK cell., both of which may be activated and/or expanded ex vivo. In some examples, the T lymphocyte or NK cell is an autologous T lymphocyte or an autologous NK cell isolated from a patient (e.g., a human patient) having a cancer. In some examples, the T lymphocyte or NK cell is an allogenic T lymphocyte or an allogenic NK cell. The T lymphocyte may be an allogeneic T lymphocyte, in which the expression of the endogenous T cell receptor has been inhibited or eliminated. Alternatively or in addition, the T lymphocyte can be activated in the presence of one or more agents selected from the group consisting of anti-CD3/CD28, IL-2, and phytohemoagglutinin. The NK cell can be activated in the presence of one or more agents selected from the group consisting of CD137 ligand protein, CD137 antibody, IL-15 protein, IL-15 receptor antibody, IL-2 protein, IL-12 protein, IL-21 protein, and K562 cell line.

In yet another aspect, described herein are pharmaceutical compositions that comprise (a) any of the nucleic acids or host cells described herein, and (b) a pharmaceutically acceptable carrier. In some examples, the composition may further comprise an Fc-containing protein such as an antibody (e.g., an IgG antibody) or an Fc-fusion protein. In some examples, the antibody is cytotoxic to cancer cells. Such an antibody may comprise a human or humanized Fc portion which binds to human CD16 (FCGR3A). Therapeutic antibody, including, but not limited to, Adalimumab, Ado-Trastuzumab emtansine, Alemtuzumab, Basiliximab, Bevacizumab, Belimumab, Brentuximab, Canakinumab, Cetuximab, Daclizumab, Denosumab, Dinutuximab, Eculizumab, Efalizumab, Epratuzumab, Gemtuzumab, Golimumab, Infliximab, Ipilimumab, Labetuzumab, Natalizumab, Obinutuzumab, Ofatumumab, Omalizumab, Palivizumab, Panitumumab, Pertuzumab, Ramucirumab, Ritutimab, Tocilizumab, Tratuzumab, Ustekinumab, or Vedolizumab.

Also provided herein are kits comprising (a) a first pharmaceutical composition that comprises any of the nucleic acids or host cells described herein, and a pharmaceutically acceptable carrier; and (b) a second pharmaceutical composition that comprises an Fc-containing protein such as an antibody (e.g., an IgG antibody) or an Fc-fusion protein (e.g., those described herein) and a pharmaceutically acceptable carrier.

Further, the present disclosure provides methods for enhancing antibody-dependent cell-mediated cytotoxicity (ADCC) in a subject. The method comprises administering to a subject in need of the treatment (e.g., a human cancer patient) an effective amount of host cells that express any of the chimeric receptors provided herein. In some embodiments, the host cells are immune cells such as natural killer cells, macrophages, neutrophils, eosinophils, T cells, or a combination thereof. In some examples, the host immune cells are autologous. In other examples, the host immune cells are allogeneic. Any of the host immune cells may be activated, expanded, or both ex vivo.

The subject may be subjected to treatment by an anti-cancer antibody, which may comprise a human or humanized Fc portion that binds to human CD16. The subject may be a patient having a cancer, such as carcinoma, lymphoma, sarcoma, blastomas, and leukemia. For example, the patient may have a cancer of B-cell origin, breast cancer, gastric cancer, neuroblastoma, osteosarcoma, lung cancer, melanoma, prostate cancer, colon cancer, renal cell carcinoma, ovarian cancer, rhabdomyosarcoma, leukemia, and Hodgkin's lymphoma. Cancers of B-cell origin include, but not limited to, B-lineage acute lymphoblastic leukemia, B-cell chronic lymphocytic leukemia, and B-cell non-Hodgkin's lymphoma.

In another aspect, the present disclosure is related to methods for enhancing efficacy of an antibody-based immunotherapy. The method comprises administering an effective amount of host cells that express any of the chimeric receptors provided herein to a subject who has been treated or is being treated with a therapeutic antibody (e.g., any of the therapeutic antibodies described herein). Exemplary host immune cells include, but are not limited to, natural killer cells, macrophages, neutrophils, eosinophils, T cells, or a combination thereof. In some examples, the host immune cells are autologous. In other examples, the host immune cells are allogeneic. Any of the host immune cells may be activated, expanded, or both ex vivo.

In some examples, the host cells bearing the chimeric receptor are co-administered with an Fc-containing protein, e.g., those described herein. In some examples, host cells bearing the chimeric receptor are administered before or after the Fc-containing protein. In some examples, host cells bearing the chimeric receptor are administered first and Fc-containing protein is subsequently administered stepwise to increase concentration until a therapeutic response is observed.

In any of the methods provided herein, the subject may be a human patient suffering from a cancer and the therapeutic antibody is for treating the cancer. In some examples, the cancer is lymphoma, breast cancer, gastric cancer, neuroblastoma, osteosarcoma, lung cancer, skin cancer, prostate cancer, colon cancer, renal cell carcinoma, ovarian cancer, rhabdomyosarcoma, leukemia, mesothelioma, pancreatic cancer, head and neck cancer, retinoblastoma, glioma, glioblastoma, or thyroid cancer.

Also within the scope of the present disclosure are (a) pharmaceutical compositions for use in enhancing ADCC activity and/or enhancing efficacy of antibody therapy of subject in need, such as a human cancer patient, the pharmaceutical composition comprising immune cells as described herein that express any of the chimeric receptor constructs described herein and a pharmaceutically acceptable carrier; and (b) use of such immune cells for manufacturing a medicament for use in the intended treatment. Any of the pharmaceutical compositions may further comprise or be co-used with an Fc-containing therapeutic agent, such as an antibody or an Fc-fusion protein.

Further, present disclosure provides methods for preparing immune cells expressing a chimeric receptor as described herein. The method comprises (i) providing a population of immune cells; (ii) introducing into the immune cells a vector (e.g., a viral vector such as a lentiviral vector or a retroviral vector, a transposon or a vector that contains a transposon sequence) or a naked nucleic acid (e.g., an mRNA) encoding any of the chimeric receptors provided herein; and (iii) culturing the immune cells under conditions allowing for expression of the chimeric receptor. Such a method may further comprise (iv) activating the immune cells expressing the chimeric receptor. In examples in which the immune cells comprise T cells, the T cells may be activated in the presence of one or more of anti-CD3 antibody, anti-CD28 antibody, IL-2, and phytohemoagglutinin. The T cells may be engineered such that the expression of the endogenous T cell receptors are inhibited or eliminated. In examples in which the immune cells comprise natural killer cells, the natural killer cells may be activated in the presence of one or more of 4-1BB ligand, anti-4-1BB antibody, IL-15, anti-IL-15 receptor antibody, IL-2, IL-12, IL-21 and K562 cells.

In some embodiments, the population of immune cells is derived from peripheral blood mononuclear cells (PBMC). Exemplary immune cells include, but are not limited to, natural killer cells, macrophages, neutrophils, eosinophils, T cells, or a combination thereof. In some embodiments, the immune cells (e.g., PBMCs) are derived from a human cancer patient. In some embodiments, the immune cells are derived from a human donor. In some embodiments, the immune cells are differentiated from stem cells or stem-like cells derived from a human patient or a human donor. In some embodiments, the immune cells are established cell lines such as NK-92 cells.

In any of the methods provided herein, the vector may be introduced into the immune cells by lentiviral transduction, retroviral transduction, DNA electroporation, or RNA electroporation. In other examples, an RNA molecule encoding a chimeric receptor described herein may be introduced into the immune cells for expression.

The details of one of more embodiments of the disclosure are set forth in the description below. Other features or advantages of the present disclosure will be apparent from the detailed description of several embodiments and also from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 is a diagram demonstrating expression of CD16V-BB-ζ receptors in T cells. A: a schematic representation of the CD16V-BB-ζ receptor construct. B: graphs showing expression of CD16V-BB-ζ receptors in peripheral blood T lymphocytes. Flow cytometric dot plots illustrate expression of CD16 (B73.1 antibody) in combination with GFP or CD3ζ in activated T lymphocytes transduced with a vector containing GFP alone (Mock) or GFP an CD16V-BB-ζ. Percentage of positive cells in each quadrant is shown. C: a photo showing a representative Western blot of cell lysates from T lymphocytes transduced with GFP alone or CD16V-BB-ζ. The membranes were probed with an anti-CD3ζ antibody.

FIG. 2 shows expression of CD16V-BB-ζ receptor in T-cell subsets. A: activated CD3+ T lymphocytes were transduced with a vector containing GFP alone (Mock) and with a vector containing the CD16V-BB-ζ construct. Expression of CD16 was tested in CD4+ and CD8+ cells by flow cytometry. Dot plots show results of one representative experiment. B: a summary of results (mean±SD) obtained with T lymphocytes from 3 donors (P=N.S.).

FIG. 3 demonstrates antibody-binding capacity of CD16V-BB-ζ receptors. A: T lymphocytes transduced with a vector containing GFP (Mock) or GFP and CD16V-BB-ζ and incubated with Rituximab for 30 minutes; the amount of antibody bound to the cell surface was visualized with a goat-anti human IgG antibody conjugated to phycoerythrin (GAH IgG) and flow cytometry. B: Jurkat cells transduced with CD16V-BB-ζ (V158) or CD16F-BB-ζ (F158) incubated with Rituximab for 30 minutes. The plot compares the relationship between mean fluorescence intensity (MFI) of GFP and MFI of GAH IgG obtained with cells expressing the two receptors. C: Jurkat cells mock-transduced or transduced with CD16V-BB-ζ co-cultured with Daudi cells labeled with calcein AM orange-red in the presence of Rituximab. Cell aggregates are quantified in the upper right quadrants of each dot plot. D: a summary of the aggregation assays illustrated in panel C. Bars show mean±SD of 3 experiments. Aggregation measured with Jurkat cells transduced with CD16V-BB-ζ in the presence of Rituximab (“Ab”) was significantly higher than that measured in the 3 other culture conditions (P<0.001 by t test).

FIG. 4 shows the relative capacity of CD16V-BB-ζ and CD16F-BB-ζ receptors to bind Trastuzumab and human IgG. Jurkat cells transduced with CD16V-BB-ζ (V158; black symbols) or CD16F-BB-ζ (F158; white symbols) were incubated with Trastuzumab or human IgG for 30 minutes. The plots compare the relation between mean fluorescence intensity (MFI) of GFP and MFI of goat-anti human (GAH) IgG conjugated to PE obtained with cells expressing either receptor (P<0.0001 for both Trastuzumab and IgG).

FIG. 5 demonstrates that immunoglobulin binding to CD16V-BB-ζ receptors induces T cell activation, exocytosis of lytic granules, and cell proliferation. A: T lymphocytes transduced with a vector containing GFP (Mock) or GFP and CD16V-BB-ζ were cultured in microtiter plates coated with Rituximab for 48 hours without IL-2; expression of CD25 was measured by flow cytometry. B: a summary of the results of the test illustrated in A, bars show CD25 expression in GFP+ cells (mean±SD of experiments with T cells from 3 donors); CD25 expression was significantly higher in T lymphocytes transduced with CD16V-BB-ζ in the presence of Rituximab (“Ab”) than in the other experimental conditions (P≤0.003). C: T lymphocytes from 4 donors transduced with a vector containing GFP (Mock) or GFP and CD16V-BB-ζ were cultured as in A (n=3) or with Daudi cells (n=3) for 4 hours; CD107a staining was measured by flow cytometry. Bars show mean±SD of the 6 experiments; CD107a expression was significantly higher in T lymphocytes transduced with CD16V-BB-ζ in the presence of Rituximab (“Ab”) than in the other experimental conditions (P<0.0001). D: mock- or CD16V-BB-ζ transduced T lymphocytes cultured alone, or with Rituximab with or without Daudi cells for up to 4 weeks. Symbols indicate percentage of cell recovery as compared to the number of input cells (mean±SD of experiments with T cells from 3 donors).

FIG. 6 demonstrates antibody-dependent cell cytotoxicity mediated by CD16V-BB-ζ T lymphocytes in vitro. A: representative examples of cytotoxicity against cancer cell lines mediated by mock- or CD16V-BB-ζ transduced T lymphocytes in the presence of the corresponding antibody. Each symbol indicates the mean of triplicate cultures (P<0.01 by paired t test for all 3 comparisons). The full set of data is shown in FIG. 7. B: Cytotoxicity of mock- or CD16V-BB-ζ transduced T lymphocytes, with or without Rituximab (“Ab”), against primary cells from patients with chronic lymphocytic leukemia (CLL). Each bar (with a different shade for each patient) corresponds to mean (±SD) cytotoxicity as determined in triplicate 4-hour assays at 2:1 E:T ratio. Cytotoxicity with CD16V-BB-ζ T cells and antibody was significantly higher than that measured in any of the other 3 conditions (P<0.0001 by t test); with mock-transduced T cells, the addition of antibody increased cytotoxicity (P=0.016); all other comparisons: P>0.05. C: cytotoxicity against the same CLL samples tested in panel B after 24 hours at 1:2 E:T in the presence of mesenchymal stromal cells (MSC). Each bar corresponds to the average of two tests. Cytotoxicity with CD16V-BB-ζ T cells plus antibody was significantly higher than that with antibody alone (P=0.0002) or cells alone (P<0.0001); cytotoxicity with antibody alone was significantly higher than that with cells alone (P=0.0045).

FIG. 7 shows the collective results of 4-hour in vitro cytotoxicity assays. Mock- or CD16V-BB-ζ T lymphocytes were cocultured with the cell lines shown and either non-reactive human immunoglobulin (“No Ab”) or the corresponding antibody (“Ab”). This was Rituximab for Daudi and Ramos, Trastuzumab for MCF-7, SKBR-3, and MKN-7, and hu14.18K322A for CHLA-255, NB1691, SK-N-SH and U-2 OS. Shown are cytotoxicities at 2:1 ratio (4:1 for CHLA-255) as compared to tumor cells cultured without T cells and/or antibody. Results correspond to mean (±SD) cytotoxicity of triplicate experiments performed with T lymphocytes of 3 donors for NB1691 and SK-BR-3, and of 1 donor for the remaining cell lines; results of Daudi are mean (±SD) cytotoxicity of triplicate measurements from 2 donors and single measurements with T lymphocytes from 4 additional donors. Mean cytotoxicity of Rituximab, Trastuzumab or hu14.18K322A when added to cultures in the absence of T cells was <10%.

FIG. 8 demonstrates that cytotoxicity of CD16V-BB-ζ T lymphocytes is powerful, specific and is not affected by unbound IgG. A: CD16V-BB-ζ T lymphocytes cocultured with the neuroblastoma cell line NB1691 with either non-reactive human immunoglobulin (“No Ab”) or the hu14.18K322A antibody (“Ab”) for 24 hours. Results correspond to mean (±SD) cytotoxicity of triplicate experiments. Cytotoxicity remained significantly higher with CD16V-BB-ζ cells plus hu14.18K322A antibody as compared to CD16V-BB-ζ T cells alone even at 1:8 E:T (P =0.0002). B: mock- or CD16V-BB-ζ-transduced T lymphocytes cocultured with the B-cell lymphoma cell line Daudi for 4 hours at 2:1 E:T in the presence of Rituximab or the non-reactive antibodies Trastuzumab or hu14.18K322A. Results correspond to mean (±SD) cytotoxicity of triplicate experiments (“Mock” results are the aggregate of triplicate experiments with each antibody). Cytotoxicity with Rituximab was significantly higher than those in all other experimental conditions (P<0.0001 for all comparisons). C: cytotoxicity of T lymphocytes expressing CD16V-BB-ζ against tumor cell lines at 8 : 1 E : T in the presence of various concentrations of immunotherapeutic antibodies and competing unbound IgG (added simultaneously to the antibody). Symbols correspond to mean (±SD) of at least triplicate measurement for each antibody concentration. For each cell line, cytotoxicities were not statistically different, regardless of the amount of unbound IgG present.

FIG. 9 demonstrates that T lymphocytes expressing CD16V-BB-ζ receptors exert anti-tumor activity in vivo. NOD-SCID-IL2RGnull mice were injected i.p. with 3×10⁵ Daudi cells labeled with luciferase. Rituximab (150 μg) was injected i.p. once weekly for 4 weeks starting on day 4. In 4 mice, no other treatment was given, while in 5 other mice, the first Rituximab injection was followed by T lymphocytes expressing CD16V-BB-ζ receptors (1×10⁷ i.p.; n=5) on days 5 and 6; other 2 groups of 4 mice each received CD16V-BB-ζ T lymphocytes preceded by i.p. injection of RPMI-1640 instead of Rituximab, or i.p. injection of RPMI-1640 medium only (“Control”). A: results of in vivo imaging of tumor growth. Each symbol corresponds to one bioluminescence measurement; lines connect all measurements in one mouse. B: representative mice (2 per group) are shown for each experimental condition. Ventral images on day 3 were processed with enhanced sensitivity to demonstrate the presence of tumors in mice of the CD16V-BB-ζ+Rituximab group. Mice were euthanized when bioluminescence reached 5×10¹⁰ photons/second. C: overall survival comparisons of mice in the different treatment groups.

FIG. 10 confirms that T lymphocytes expressing CD16V-BB-ζ receptors exert anti-tumor activity in vivo. NOD-SCID-IL2RGnull mice were injected i.p. with 3×10⁵ NB1691 cells labeled with luciferase. Hu14.18K322A antibody (25 μg) was injected i.p. once weekly for 4 weeks starting on day 5. In 4 mice, no other treatment was given, while in 4 other mice, the first antibody injection was followed by T lymphocytes expressing CD16V-BB-ζ receptors (1×10⁷ i.p.; n=4) on days 6 and 7; other 2 groups of 4 mice each received CD16V-BB-ζ T lymphocytes preceded by i.p. injection of RPMI-1640 instead of antibody, or i.p. injection of RPMI-1640 medium only (“Control”). A: results of in vivo imaging of tumor growth. Each symbol corresponds to one bioluminescence measurement; lines connect all measurements in one mouse. B: images of all mice for each experimental condition. Mice were euthanized when bioluminescence reached 1×10¹⁰ photons/second. C: overall survival comparisons of mice in the different treatment groups.

FIG. 11 demonstrates functional differences between T lymphocytes expressing CD16V-BB-ζ and CD16F-BB-ζ receptors. A: flow cytometric dot plots show expression of CD16 (detected with the B73.1 antibody) and green fluorescent protein (GFP) in T lymphocytes transduced with CD16V-BB-ζ or CD16F-BB-ζ. Percentage of positive cells in each quadrant is shown. B: T lymphocytes transduced with either CD16V or CD16F receptor were cultured with Daudi, SK-BR-3 or NB1691 cells in the presence of Rituximab, Trastuzumab and hu14.18K322A, respectively. All antibodies were used at 0.1 μg/mL. Symbols indicate percentage of cell recovery as compared to the number of input cells (mean±SD of 3 experiments); cell counts for weeks 1-3 of culture were significantly different by paired t test for all 3 cultures (Daudi, P=0.0007; SK-BR-3, P=0.0164; NB1691, P=0.022). C: antibody-dependent cell cytotoxicity mediated by T lymphocytes expressing either CD16V-BB-ζ or CD16F-BB-ζ receptors against Daudi cells in the presence of various concentrations of Rituximab. Each symbol indicates the mean±SD of triplicate cultures at 8:1 (left) or 2:1 (right) E:T. Cytotoxicities of T cells with CD16V-BB-ζ were significantly higher than those of T cells with CD16F-BB-ζ (P<0.001 for either E:T).

FIG. 12 shows a schematic representation of CD16 chimeric receptors used in this study.

FIG. 13 shows expression of CD16V receptors with different signaling domains. Flow cytometric dot plots illustrate expression of CD16 (detected with the 3G8 antibody) in combination with GFP in activated T lymphocytes transduced with a vector containing green fluorescent protein (GFP) alone (Mock) or different CD16V constructs. Percentage of positive cells in each quadrant is shown.

FIG. 14 demonstrates that CD16V-BB-ζ induces higher T cell activation, proliferation and cytotoxicity than CD16V receptors with different signaling properties. A: CD25 mean fluorescence intensity (MFI) by flow cytometry plotted against green fluorescent protein (GFP) MFI in T lymphocytes expressing different chimeric receptors after 48-hour co-culture with Daudi cells and Rituximab (0.1 μg/mL). CD25 expression with CD16V-BB-ζ was significantly higher than that triggered by CD16V-ζ, CD16V-FcϵRIγ or CD16V with no signaling capacity (“CD16V-trunc.”) (P<0.0001 by linear regression analysis). B: T lymphocytes transduced with various CD16V receptors were cultured with Daudi, SK-BR-3 or NB1691 cells in the presence of Rituximab, Trastuzumab and hu14.18K322A, respectively. All antibodies were used at 0.1 μg/mL. Symbols indicate percentage of cell recovery as compared to number of input cells (mean±SD of 3 experiments); cell counts for weeks 1-3 of culture were significantly higher with CD16V-BB-ζ receptors that with all other receptors by paired t test for all 3 cultures (P<0.0001). C: ADCC of T lymphocytes expressing various CD16V receptors or mock-transduced T cells against Daudi, SK-BR-3 and NB1691 in the presence of Rituximab, Trastuzumab and hu14.18K322A, respectively. Symbols are mean±SD of triplicate cultures at the E:T shown. Cytotoxicities with CD16V-BB-ζ receptors were significantly higher than those with all other receptors (P<0.0001 by t test in all comparisons) while cytotoxicities of lymphocytes mock-transduced or transduced with the CD16V-truncated receptor were not significantly different (P>0.05) from each other; cytotoxicity with CD16V-FcϵRIϵ was significantly higher than that with CD3-ζ against Daudi (P=0.006) and SK-BR-3 (P=0.019); lymphocytes expressing either receptor had higher cytotoxicities than those mock-transduced or transduced with CD16V-truncated (P<0.01 for all comparisons).

FIG. 15 demonstrates expression of CD16V-BB-ζ receptors by mRNA electroporation. A: activated T lymphocytes were electroporated with CD16V-BB-ζ mRNA or without mRNA (Mock); expression of CD16 was tested 24 hours later by flow cytometry. B: cytotoxicity of mock or CD16V-BB-ζ electroporated T cells was tested against the Ramos cell line in the presence of Rituximab. Symbols show mean±SD percent cytotoxicity (n=3; P<0.01 for comparisons at all E:T ratios).

FIG. 16 shows binding of Rituxan to Jurkat cells with and without expression of the chimeric receptor SEQ ID NO: 1. Jurkat cells were electroporated in the presence of no mRNA (panel A) or mRNA encoding chimeric receptor SEQ ID NO: 1 (panel B), incubated with Rituxan, stained with a PE-labeled goat-anti-human antibody to detect bound Rituxan, and analyzed by flow cytometry. In panels A and B, the same quadrant gate was applied to each set of data and the percentage of cells in each quadrant is shown, with the top right quadrant representing Rituxan-positive cells. In panel C, cell number is plotted as a function of Rituxan staining for mock-electroporated cells (no fill) and cells electroporated with mRNA encoding chimeric receptor SEQ ID NO: 1 (gray).

FIG. 17 shows the presence of CD25 on Jurkat cells, with or without expression of chimeric receptor SEQ ID NO: 1, in the presence of Rituxan and target Daudi cells. Jurkat cells were electroporated in the presence of no mRNA (panel A) or mRNA encoding chimeric receptor SEQ ID NO: 1(panel 17B), and subsequently incubated with Rituxan and target Daudi cells. Cells were stained with a PE-labeled anti-CD7 antibody to isolate Jurkat cells and APC-labeled anti-C25 antibody to detect CD25 expression, and analyzed by flow cytometry. CD7-positive cells are shown in panels A and B, and the same quadrant gate was applied to each set of data. The percentage of cells in each quadrant is shown, with the top right quadrant representing CD25-positive cells. Panel C shows a histogram of data from the same experiment. The number of CD7-positive cells is plotted as a function of CD25 staining for mock-electroporated cells (no fill) and cells electroporated with mRNA encoding chimeric receptor SEQ ID NO: 1(gray) are plotted as a function of CD25 staining.

FIG. 18 shows the presence of CD69 on Jurkat cells, with or without expression of chimeric receptor SEQ ID NO: 1, in the presence of Rituxan and target Daudi cells. Jurkat cells were electroporated in the presence of no mRNA (panel A) or mRNA encoding SEQ ID NO: 1 (panel B), and subsequently incubated with Rituxan and target Daudi cells. Cells were stained with a PE-labeled anti-CD7 antibody to isolate Jurkat cells and APC-labeled anti-C69 antibody to detect CD69 expression, and analyzed by flow cytometry. CD7-positive cells are shown in panels A and B, and the same quadrant gate was applied to each set of data. The percentage of cells in each quadrant is shown, with the top right quadrant representing CD69-positive cells. Panel C shows a histogram of data from the same experiment. The number of CD7-positive cells is plotted as a function of CD69 staining for mock-electroporated cells (no fill) and cells electroporated with mRNA encoding chimeric receptor SEQ ID NO: 1 (gray).

FIG. 19 shows a representative anti-CD3ζ western blot analysis of chimeric receptors. Jurkat cells were electroporated without mRNA (lane 1) or with mRNA encoding chimeric receptor SEQ ID NO: 1 (lane 2), SEQ ID NO: 3 (lane 3), SEQ ID NO: 10 (lane 4), SEQ ID NO: 11 (lane 5), SEQ ID NO: 14 (lane 6), SEQ ID NO: 2 (lane 7), SEQ ID NO: 4 (lane 8), SEQ ID NO: 5 (lane 9), SEQ ID NO: 7 (lane 10), SEQ ID NO: 8 (lane 11), SEQ ID NO: 9 (lane 12), or SEQ ID NO: 6 (lane 13). Cells were harvested, lysed, and analyzed by Western blot analysis with an anti-CD3ζ antibody. Chimeric receptor proteins were detected in lysates from all cells that were electroporated with chimeric receptor mRNA.

### DETAILED DESCRIPTION OF DISCLOSURE

Antibody-based immunotherapies are used to treat a wide variety of diseases, including many types of cancer. Such a therapy often depends on recognition of cell surface molecules that are differentially expressed on cells for which elimination is desired (e.g., target cells such as cancer cells) relative to normal cells (e.g., non-cancer cells) (Weiner et al. Cell (2012) 148(6): 1081-1084). Several antibody-based immunotherapies have been shown in vitro to facilitate antibody-dependent cell-mediated cytotoxicity of target cells (e.g. cancer cells), and for some it is generally considered that this is the mechanism of action in vivo, as well. ADCC is a cell-mediated innate immune mechanism whereby an effector cell of the immune system, such as natural killer (NK) cells, T cells, monocyte cells, macrophages, or eosinophils, actively lyses target cells (e.g., cancer cells) recognized by specific antibodies.

The chimeric receptors described herein would confer a number of advantages. For example, via the extracellular domain that binds Fc, the chimeric receptor constructs described herein can bind to the Fc portion of antibodies or other Fc-containing molecules, rather than directly binding a specific target antigen (e.g., a cancer antigen). Thus, immune cells expressing the chimeric receptor constructs described herein would be able to induce cell death of any type of cells that are bound by an antibody or another Fc-containing molecule.

The present disclosure provides chimeric receptors capable of binding to Fc-containing molecules (e.g., antibodies or Fc fusion proteins), immune cells expressing such, and methods of using the immune cells to enhance ADCC effects against target cells (e.g., cancer cells). As used herein, a chimeric receptor refers to a non-naturally occurring molecule that can be expressed on the surface of a host cell and comprises an extracellular domain capable of binding to a target molecule containing an Fc portion and one or more cytoplasmic signaling domains for triggering effector functions of the immune cell expressing the chimeric receptor, wherein at least two domains of the chimeric receptor are derived from different molecules.

Fc-containing molecules such as antibodies proteins can bind to a target such as a cell surface molecule, receptor, or carbohydrate on the surface of a target cell (e.g., a cancer cell). Immune cells that express receptors capable of binding such Fc-containing molecules, for example the chimeric receptor molecules described herein, recognize the target cell-bound antibodies and this receptor/antibody engagement stimulates the immune cell to perform effector functions such as release of cytotoxic granules or expression of cell-death-inducing molecules, leading to cell death of the target cell recognized by the Fc-containing molecules.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

In the context of the present disclosure insofar as it relates to any of the disease conditions recited herein, the terms “treat”, “treatment”, and the like mean to relieve or alleviate at least one symptom associated with such condition, or to slow or reverse the progression of such condition. Within the meaning of the present disclosure, the term “treat” also denotes to arrest, delay the onset (i.e., the period prior to clinical manifestation of a disease) and/or reduce the risk of developing or worsening a disease. For example, in connection with cancer the term “treat” may mean eliminate or reduce a patient's tumor burden, or prevent, delay or inhibit metastasis, etc.

As used herein the term “therapeutically effective” applied to dose or amount refers to that quantity of a compound or pharmaceutical composition (e.g., a composition comprising immune cells such as T lymphocytes and/or NK cells) comprising a chimeric receptor of the disclosure, and optionally further comprising a tumor-specific cytotoxic monoclonal antibody or another anti-tumor molecule comprising the Fc portion (e.g., a composite molecule constituted by a ligand (e.g., cytokine, immune cell receptor) binding a tumor surface receptor combined with the Fc-portion of an immunoglobulin or Fc-containing DNA or RNA)) that is sufficient to result in a desired activity upon administration to a subject in need thereof. Within the context of the present disclosure, the term “therapeutically effective” refers to that quantity of a compound or pharmaceutical composition that is sufficient to delay the manifestation, arrest the progression, relieve or alleviate at least one symptom of a disorder treated by the methods of the present disclosure. Note that when a combination of active ingredients is administered the effective amount of the combination may or may not include amounts of each ingredient that would have been effective if administered individually.

The phrase “pharmaceutically acceptable”, as used in connection with compositions of the present disclosure, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

As used herein, the term “subject” refers to any mammal. In a preferred embodiment, the subject is human.

As used in this specification and the appended claims, the singular forms “a,” “an,” and “the” include plural references unless the context clearly dictates otherwise.

### I. Chimeric Receptors

The chimeric receptors described herein comprise an extracellular domain with binding affinity and specificity for the Fc portion of an immunoglobulin (“Fc binder”), a transmembrane domain, at least one co-stimulatory signaling domain, and a cytoplasmic signaling domain comprising an ITAM. The chimeric receptors are configured such that, when expressed on a host cell, the extracellular ligand-binding domain is located extracellularly for binding to a target molecule (e.g., an antibody or a Fc-fusion protein) and the co-stimulatory signaling domain and the ITAM-containing cytoplasmic signaling domain are located in the cytoplasm for triggering activation and/or effector signaling. In some embodiments, a chimeric receptor construct as described herein comprises, from N-terminus to C-terminus, the Fc binder, the transmembrane domain, the at least one co-stimulatory signaling domain, and the ITAM-containing cytoplasmic signaling domain. In other embodiments, a chimeric receptor construct as described herein comprises, from N-terminus to C-terminus, the Fc binder, the transmembrane domain, the ITAM-containing cytoplasmic signaling domains, and the at least one co-stimulatory signaling domain.

Any of the chimeric receptors described herein may further comprise a hinge domain, which may be located at the C-terminus of the Fc binder and the N-terminus of the transmembrane domain. Alternatively or in addition, the chimeric receptor constructs described herein may contain two or more co-stimulatory signaling domains, which may link to each other or be separated by the ITAM-containing cytoplasmic signaling domain. The extracellular Fc binder, transmembrane domain, co-stimulatory signaling domain(s), and ITAM-containing cytoplasmic signaling domain in a chimeric receptor construct may be linked to each other directly, or via a peptide linker. In some embodiments, any of the chimeric receptors described herein comprises a signal sequence at the N-terminus.

A. Fc binders

The chimeric receptor constructs described herein comprises an extracellular domain that is an Fc binder, i.e., capable of binding to the Fc portion of an immunoglobulin (e.g., IgG, IgA, IgM, or IgE) of a suitable mammal (e.g., human, mouse, rat, goat, sheep, or monkey). Suitable Fc binders may be derived from naturally occurring proteins such as mammalian Fc receptors or certain bacterial proteins (e.g., protein A, protein G). Additionally, Fc binders may be synthetic polypeptides engineered specifically to bind the Fc portion of any of the Ig molecules described herein with high affinity and specificity. For example, such an Fc binder can be an antibody or an antigen-binding fragment thereof that specifically binds the Fc portion of an immunoglobulin. Examples include, but are not limited to, a single-chain variable fragment (scFv), a domain antibody, or a nanobody. Alternatively, an Fc binder can be a synthetic peptide that specifically binds the Fc portion, such as a Kunitz domain, a small modular immunopharmaceutical (SMIP), an adnectin, an avimer, an affibody, a DARPin, or an anticalin, which may be identified by screening a peptide combinatory library for binding activities to Fc.

In some embodiments, the Fc binder is an extracellular ligand-binding domain of a mammalian Fc receptor. As used herein, an “Fc receptor” is a cell surface bound receptor that is expressed on the surface of many immune cells (including B cells, dendritic cells, natural killer (NK) cells, macrophage, neutorphils, mast cells, and eosinophils) and exhibits binding specificity to the Fc domain of an antibody. Fc receptors are typically comprised of at least 2 immunoglobulin (Ig)-like domains with binding specificity to an Fc (fragment crystallizable) portion of an antibody. In some instances, binding of an Fc receptor to an Fc portion of the antibody may trigger antibody dependent cell-mediated cytotoxicity (ADCC) effects. The Fc receptor used for constructing a chimeric receptor as described herein may be a naturally-occurring polymorphism variant (e.g., the CD16 V158 variant), which may have increased or decreased affinity to Fc as compared to a wild-type counterpart. Alternatively, the Fc receptor may be a functional variant of a wild-type counterpart, which carry one or more mutations (e.g., up to 10 amino acid residue substitutions) that alter the binding affinity to the Fc portion of an Ig molecule. In some instances, the mutation may alter the glycosylation pattern of the Fc receptor and thus the binding affinity to Fc.

The table below lists a number of exemplary polymorphisms in Fc receptor extracellular domains (see, e.g., Kim et al., J. Mol. Evol. 53:1-9, 2001):

TABLE 1
Exemplary Polymorphisms in Fc Receptors

| | Amino Acid | | | | | | | | | |
|---|---|---|---|---|---|---|---|---|---|---|
| | Number | | | | | | | | | |
| | 19 | 48 | 65 | 89 | 105 | 130 | 134 | 141 | 142 | 158 |
| FCR10 | R | S | D | I | D | G | F | Y | T | V |
| P08637 | R | S | D | I | D | G | F | Y | I | F |
| S76824 | R | S | D | I | D | G | F | Y | I | V |
| J04162 | R | N | D | V | D | D | F | H | I | V |
| M31936 | S | S | N | I | D | D | F | H | I | V |
| M24854 | S | S | N | I | E | D | S | H | I | V |
| X07934 | R | S | N | I | D | D | F | H | I | V |
| X14356 (FcγRII) | N | N | N | S | E | S | S | S | I | I |
| M31932 (FcγRI) | S | T | N | R | E | A | F | T | I | G |
| X06948 (FcαϵI) | R | S | E | S | Q | S | E | S | I | V |

Fc receptors are classified based on the isotype of the antibody to which it is able to bind. For example, Fc-gamma receptors (FcyR) generally bind to IgG antibodies, such as one or more subtype thereof (i.e., IgG1, IgG2, IgG3, IgG4); Fc-alpha receptors (FcaR) generally bind to IgA antibodies; and Fc-epsilon receptors (FccR) generally bind to IgE antibodies. In some embodiments, the Fc receptor is an Fc-gamma receptor, an Fc-alpha receptor, or an Fc-epsilon receptor. Examples of Fc-gamma receptors include, without limitation, CD64A, CD64B, CD64C, CD32A, CD32B, CD16A, and CD16B. An example of an Fc-alpha receptor is FcaRl/CD89. Examples of Fc-epsilon receptors include, without limitation, FcϵRI and FcϵRII/CD23. The table below lists exemplary Fc receptors for use in constructing the chimeric receptors described herein and their binding activity to corresponding Fc domains:

TABLE 2
Exemplary Fc Receptors

| | Principal | |
|---|---|---|
| Receptor name | antibody ligand | Affinity for ligand |
| FcγRI (CD64) | IgG1 and IgG3 | High (Kd~10⁻⁹M) |
| FcγRIIA (CD32) | IgG | Low (Kd >10⁻⁷M) |
| FcγRIIB1 (CD32) | IgG | Low (Kd >10⁻⁷M) |
| FcγRIIB2 (CD32) | IgG | Low (Kd >10⁻⁷M) |
| FcγRIIIA (CD16a) | IgG | Low (Kd >10⁻⁶M) |
| FcγRIIIB (CD16b) | IgG | Low (Kd >10⁻⁶M) |
| FcϵRI | IgE | High (Kd~10⁻¹⁰M) |
| FcϵRII (CD23) | IgE | Low (Kd >10⁻⁷M) |
| FcαRI (CD89) | IgA | Low (Kd >10⁻⁶M) |
| Fcα/μR | IgA and IgM | High for IgM, Mid for IgA |
| FcRn | IgG | |

Selection of the ligand binding domain of an Fc receptor for use in the chimeric receptors described herein will be apparent to one of skill in the art. For example, it may depend on factors such as the isotype of the antibody to which binding of the Fc receptor is desired and the desired affinity of the binding interaction.

In some examples, (a) is the extracellular ligand-binding domain of CD16, which may incorporate a naturally occurring polymorphism that may modulate affinity for Fc. In some examples, (a) is the extracellular ligand-binding domain of CD16 incorporating a polymorphism at position 158 (e.g., valine or phenylalanine). In some embodiments, (a) is produced under conditions that alter its glycosylation state and its affinity for Fc.

In some embodiments, (a) is the extracellular ligand-binding domain of CD16 incorporating modifications that render the chimeric receptor incorporating it specific for a subset of IgG antibodies. For example, mutations that increase or decrease the affinity for an IgG subtype (e.g., IgG1) may be incorporated.

In some examples, (a) is the extracellular ligand-binding domain of CD32, which may incorporate a naturally occurring polymorphism that may modulate affinity for Fc. In some embodiments, (a) is produced under conditions that alter its glycosylation state and its affinity for Fc.

In some embodiments, (a) is the extracellular ligand-binding domain of CD32 incorporating modifications that render the chimeric receptor incorporating it specific for a subset of IgG antibodies. For example, mutations that increase or decrease the affinity for an IgG subtype (e.g., IgG1) may be incorporated.

In some examples, (a) is the extracellular ligand-binding domain of CD64, which may incorporate a naturally occurring polymorphism that may modulate affinity for Fc. In some embodiments, (a) is produced under conditions that alter its glycosylation state and its affinity for Fc.

In some embodiments, (a) is the extracellular ligand-binding domain of CD64 incorporating modifications that render the chimeric receptor incorporating it specific for a subset of IgG antibodies. For example, mutations that increase or decrease the affinity for an IgG subtype (e.g., IgG1) may be incorporated.

In other embodiments, the Fc binder is derived from a naturally occurring bacterial protein that is capable of binding to the Fc portion of an IgG molecule. A Fc binder for use in constructing a chimeric receptor as described herein can be a full-length protein or a functional fragment thereof. Protein A is a 42 kDa surface protein originally found in the cell wall of the bacterium Staphylococcus aureus. It is composed of five domains that each fold into a three-helix bundle and are able to bind IgG through interactions with the Fc region of most antibodies as well as the Fab region of human VH3 family antibodies. Protein G is an approximately 60-kDa protein expressed in group C and G Streptococcal bacteria that binds to both the Fab and Fc region of mammalian IgGs. While native protein G also binds albumin, recombinant variants have been engineered that eliminate albumin binding.

Fc binders for use in chimeric receptors may also be created de novo using combinatorial biology or directed evolution methods. Starting with a protein scaffold (e.g., an scFv derived from IgG, a Kunitz domain derived from a Kunitz-type protease inhibitor, an ankyrin repeat, the Z domain from protein A, a lipocalin, a fibronectin type III domain, an SH3 domain from Fyn, or others), amino acid side chains for a set of residues on the surface may be randomly substituted in order to create a large library of variant scaffolds. From large libraries it is possible to isolate rare variants with affinity for a target like the Fc domain by first selecting for binding, followed by amplification by phage, ribosome or cell display. Repeated rounds of selection and amplification can be used to isolate those proteins with the highest affinity for the target. Fc-binding peptides are known in the art, e.g., DeLano et al., Science, 287:5456 (2000); Jeong et al., Peptides, 31(2):202-206 (2009); and Krook et al., J. Immunological Methods, 221(1-2):151-157 (1998). Exemplary Fc-binding peptides may comprise the amino acid sequence of ETQRCTWHMGELVWCEREHN (SEQ ID NO:85), KEASCSYWLGELVWCVAGVE (SEQ ID NO:86), or DCAWHLGELVWCT (SEQ ID NO:87).

Any of the Fc binders described herein may have a suitable binding affinity for the Fc portion of a therapeutic antibody. As used herein, “binding affinity” refers to the apparent association constant or K_{A}. The K_{A} is the reciprocal of the dissociation constant, K_{D}. The extracellular ligand-binding domain of an Fc receptor domain of the chimeric receptors described herein may have a binding affinity K_{D} of at least 10⁻⁵, 10⁻⁶, 10⁻⁷, 10⁻⁸, 10⁻⁹, 10⁻¹⁰M or lower for the Fc portion of antibody. In some embodiments, the Fc binder has a high binding affinity for antibody, isotype of antibodies, or subtype(s) thereof, as compared to the binding affinity of the Fc binder to another antibody, isotype of antibodies or subtypes thereof. In some embodiments, the extracellular ligand-binding domain of an Fc receptor has specificity for an antibody, isotype of antibodies, or subtype(s) thereof, as compared to binding of the extracellular ligand-binding domain of an Fc receptor to another antibody, isotype of antibodies, or subtypes thereof. Fc-gamma receptors with high affinity binding include CD64A, CD64B, and CD64C. Fc-gamma receptors with low affinity binding include CD32A, CD32B, CD16A, and CD16B. An Fc-epsilon receptor with high affinity binding is FccRI, and an Fc-epsilon receptor with low affinity binding is FcϵRII/CD23.

The binding affinity or binding specificity for an Fc receptor or a chimeric receptor comprising an Fc binder (e.g., an extracellular ligand-binding domain of an Fc receptor) can be determined by a variety of methods including equilibrium dialysis, equilibrium binding, gel filtration, ELISA, surface plasmon resonance, or spectroscopy.

In some embodiments, the extracellular ligand-binding domain of an Fc receptor comprises an amino acid sequence that is at least 90% (e.g., 91, 92, 93, 94, 95, 96, 97, 98, 99%) identical to the amino acid sequence of the extracellular ligand-binding domain of a naturally-occurring Fc-gamma receptor, an Fc-alpha receptor, or an Fc-epsilon receptor. The “percent identity” of two amino acid sequences can be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the disclosure. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

Also within the scope of the present disclosure are variants of the extracellular ligand-binding domains of Fc receptors, such as those described herein. In some embodiments, the variant extracellular ligand-binding domain may comprise up to 10 amino acid residue variations (e.g., 1, 2, 3, 4, or 5) relative to the amino acid sequence of the reference extracellular ligand-binding domain. In some embodiments, the variant can be a naturally-occurring variant due to gene polymorphism. In other embodiments, the variant can be a non-naturally occurring modified molecule. For examples, mutations may be introduced into the extracellular ligand-binding domain of an Fc receptor to alter its glycosylation pattern and thus its binding affinity to the corresponding Fc domain.

In some examples, the Fc receptor can be CD16A, CD16B, CD32A, CD32B, CD32C, CD64A, CD64B, CD64C, or a variant thereof as described herein. The extracellular ligand-binding domain of an Fc receptor may comprise up to 10 amino acid residue variations (e.g., 1, 2, 3, 4, 5, or 8) relative to the amino acid sequence of the extracellular ligand-binding domain of CD16A, CD16B, CD32A, CD32B, CD32C, CD64A, CD64B, CD64C as described herein. Such Fc domains comprising one or more amino acid variations may be referred to as a variant. Mutation of amino acid residues of the extracellular ligand-binding domain of an Fc receptor may result in an increase in binding affinity for the Fc receptor domain to bind to an antibody, isotype of antibodies, or subtype(s) thereof relative to Fc receptor domains that do not comprise the mutation. For example, mutation of residue 158 of the Fc-gamma receptor CD16A may result in an increase in binding affinity of the Fc receptor to an Fc portion of an antibody. In some embodiments, the mutation is a substitution of a phenylalanine to a valine at residue 158 of the Fc-gamma receptor CD16A, referred to as a CD16A V158 variant. The amino acid sequence of human CD16A V158 variant is provided below with the V158 residue highlighted in bold/face (signal peptide italicized):

(SEQ ID NO: 75)
MWQLLLPTALLLLVSAGMRTEDLPKAVVFLEPQWYRVLEKDSVTLKCQGA
YSPEDNSTQWFHNESLISSQASSYFIDAATVDDSGEYRCQTNLSTLSDPV
QLEVHIGWLLLQAPRWVFKEEDPIHLRCHSWKNTALHKVTYLQNGKGRKY
FHHNSDFYIPKATLKDSGSYFCRGLVGSKNVSSETVNITITQGLAVSTIS
SFFPPGYQVSFCLVMVLLFAVDTGLYFSVKTNIRSSTRDWKDHKFKWRKD
PQDK

Alternative or additional mutations that can be made in the extracellular ligand-binding domain of an Fc receptor that may enhance or reduce the binding affinity to an Fc portion of a molecule such as an antibody will be evident to one of ordinary skill in the art. In some embodiments, the Fc receptor is CD16A, CD16A V158 variant, CD16B, CD32A, CD32B, CD32C, CD64A, CD64B, or CD64C. In some embodiments, the extracellular ligand-binding domain of the chimeric receptor constructs described herein is not the extracellular ligand-binding domain of CD16A or CD16A V158 variant.

### B. Transmembrane Domain

The transmembrane domain of the chimeric receptors described herein can be in any form known in the art. As used herein, a “transmembrane domain” refers to any protein structure that is thermodynamically stable in a cell membrane, preferably a eukaryotic cell membrane. Transmembrane domains compatible for use in the chimeric receptors used herein may be obtained from a naturally occurring protein. Alternatively, it can be a synthetic, non-naturally occurring protein segment, e.g., a hydrophobic protein segment that is thermodynamically stable in a cell membrane.

Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times).

Membrane proteins may be defined as Type I, Type II or Type III depending upon the topology of their termini and membrane-passing segment(s) relative to the inside and outside of the cell. Type I membrane proteins have a single membrane-spanning region and areoriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-termini.

In some embodiments, the transmembrane domain of the chimeric receptor described herein is derived from a Type I single-pass membrane protein. Single-pass membrane proteins include, but are not limited to, CD8α, CD8β, 4-1BB/CD137, CD28, CD34, CD4, FcϵRIγ, CD16, OX40/CD134, CD3ζ, CD3ϵ, CD3γ, CD3δ, TCRα, TCRβ, TCRζ, CD32, CD64, CD64, CD45, CDS, CD9, CD22, CD37, CD80, CD86, CD40, CD4OL/CD154, VEGFR2, FAS, and FGFR2B. In some embodiments, the transmembrane domain is from a membrane protein selected from the following: CD8α, CD8β, 4-1BB/CD137, CD28, CD34, CD4, FcϵRIγ, CD16, OX40/CD134, CD3ζ, CD3ϵ, CD3γ, CD3δ, TCRα, CD32, CD64, VEGFR2, FAS, and FGFR2B. In some examples, the transmembrane domain is of CD8a. In some examples, the transmembrane domain is of 4-1BB/CD137. In other examples, the transmembrane domain is of CD28 or CD34. In yet other examples, the transmembrane domain is not derived from human CD8α. In some embodiments, the transmembrane domain of the chimeric receptor is a single-pass alpha helix.

Transmembrane domains from multi-pass membrane proteins may also be compatible for use in the chimeric receptors described herein. Multi-pass membrane proteins may comprise a complex (at least 2, 3, 4, 5, 6, 7 or more) alpha helices or a beta sheet structure. Preferably, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side. Either one or multiple helix passes from a multi-pass membrane protein can be used for constructing the chimeric receptor described herein.

Transmembrane domains for use in the chimeric receptors described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 B1 and PCT Publication No. WO 2000/032776 A2, the relevant disclosures of which are incorporated by reference herein.

In some embodiments, the amino acid sequence of the transmembrane domain does not comprise cysteine residues. In some embodiments, the amino acid sequence of the transmembrane domain comprises one cysteine residue. In some embodiments, the amino acid sequence of the transmembrane domain comprises two cysteine residues. In some embodiments, the amino acid sequence of the transmembrane domain comprises more than two cysteine residues (e.g., 3, 4, 5 or more).

The transmembrane domain may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence.

The hydropathy, or hydrophobic or hydrophilic characteristics of a protein or protein segment, can be assessed by any method known in the art, for example the Kyte and Doolittle hydropathy analysis.

### C. Co-stimulatory Signaling Domains

Many immune cells require co-stimulation, in addition to stimulation of an antigen-specific signal, to promote cell proliferation, differentiation and survival, as well as to activate effector functions of the cell. The chimeric receptors described herein comprise at least one co-stimulatory signaling domain. The term “co-stimulatory signaling domain,” as used herein, refers to at least a portion of a protein that mediates signal transduction within a cell to induce an immune response such as an effector function. The co-stimulatory signaling domain of the chimeric receptor described herein can be a cytoplasmic signaling domain from a co-stimulatory protein, which transduces a signal and modulates responses mediated by immune cells, such as T cells, NK cells, macrophages, neutrophils, or eosinophils.

Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory molecule may be compatible for use in the chimeric receptors described herein. The type(s) of co-stimulatory signaling domain is selected based on factors such as the type of the immune cells in which the chimeric receptors would be expressed (e.g., T cells, NK cells, macrophages, neutrophils, or eosinophils) and the desired immune effector function (e.g., ADCC effect). Examples of co-stimulatory signaling domains for use in the chimeric receptors can be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g.,4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACl/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMFS, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thyl, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C. In some embodiments, the co-stimulatory signaling domain is of 4-1BB, CD28, OX40, ICOS, CD27, GITR, HVEM, TIM1, LFA1(CD11a) or CD2, or any variant thereof. In other embodiments, the co-stimulatory signaling domain is not derived from 4-1BB.

Also within the scope of the present disclosure are variants of any of the co-stimulatory signaling domains described herein, such that the co-stimulatory signaling domain is capable of modulating the immune response of the immune cell. In some embodiments, the co-stimulatory signaling domains comprises up to 10 amino acid residue variations (e.g., 1, 2, 3, 4, 5, or 8) as compared to a wild-type counterpart. Such co-stimulatory signaling domains comprising one or more amino acid variations may be referred to as variants.

Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation. For example, mutation of residues 186 and 187 of the native CD28 amino acid sequence may result in an increase in co-stimulatory activity and induction of immune responses by the co-stimulatory domain of the chimeric receptor. In some embodiments, the mutations are substitution of a lysine at each of positions 186 and 187 with a glycine residue of the CD28 co-stimulatory domain, referred to as a CD28_{LL→GG} variant. Additional mutations that can be made in co-stimulatory signaling domains that may enhance or reduce co-stimulatory activity of the domain will be evident to one of ordinary skill in the art. In some embodiments, the co-stimulatory signaling domain is of 4-1BB, CD28, OX40, or CD28_{LL→GG} variant. In some embodiments, the co-stimulatory signaling domain is not of 4-1BB.

In some embodiments, the chimeric receptors may comprise more than one co-stimulatory signaling domain (e.g., 2, 3 or more). In some embodiments, the chimeric receptor comprises two or more of the same co-stimulatory signaling domains, for example, two copies of the co-stimulatory signaling domain of CD28. In some embodiments, the chimeric receptor comprises two or more co-stimulatory signaling domains from different co-stimulatory proteins, such as any two or more co-stimulatory proteins described herein. Selection of the type(s) of co-stimulatory signaling domains may be based on factors such as the type of host cells to be used with the chimeric receptors (e.g., immune cells such as T cells, NK cells, macrophages, neutrophils, or eosinophils) and the desired immune effector function. In some embodiments, the chimeric receptor comprises two co-stimulatory signaling domains. In some embodiments, the two co-stimulatory signaling domains are CD28 and 4-1BB. In some embodiments, the two co-stimulatory signaling domains are CD28_{LL→GG} variant and 4-1BB.

### D. Cytoplasmic Signaling Domain Comprising an Immunoreceptor Tyrosine-based Activation Motif (ITAM)

Any cytoplasmic signaling domain comprising an immunoreceptor tyrosine-based activation motif (ITAM) can be used to construct the chimeric receptors described herein. An “ITAM,” as used herein, is a conserved protein motif that is generally present in the tail portion of signaling molecules expressed in many immune cells. The motif may comprises two repeats of the amino acid sequence YxxL/I separated by 6-8 amino acids, wherein each x is independently any amino acid, producing the conserved motif YxxL/Ix₍₆₋₈₎YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways. In some examples, the cytoplasmic signaling domain comprising an ITAM is of CD3ζ or FcϵR1γ. In other examples, the ITAM-containing cytoplasmic signaling domain is not derived from human CD3ζ. In yet other examples, the ITAM-containing cytoplasmic signaling domain is not derived from an Fc receptor, when the extracellular ligand-binding domain of the same chimeric receptor construct is derived from CD16A.

In one specific embodiment, several signaling domains can be fused together for additive or synergistic effect. Non-limiting examples of useful additional signaling domains include part or all of one or more of TCR Zeta chain, CD28, OX40/CD134, 4-1BB/CD137, FcϵRIγ, ICOS/CD278, ILRB/CD122, IL-2RG/CD132, and CD40.

### E. Hinge Domain

In some embodiments, the chimeric receptors described herein further comprise a hinge domain that is located between the extracellular ligand-binding domain and the transmembrane domain. A hinge domain is an amino acid segment that is generally found between two domains of a protein and may allow for flexibility of the protein and movement of one or both of the domains relative to one another. Any amino acid sequence that provides such flexibility and movement of the extracellular ligand-binding domain of an Fc receptor relative to the transmembrane domain of the chimeric receptor can be used.

The hinge domain may contain about 10-100 amino acids, e.g., 15-75 amino acids, 20-50 amino acids, or 30-60 amino acids. In some embodiments, the hinge domain may be of 10, 11, 12, 13, 14,15, 16, 17, 18, 19, 20, 21, 22, 23, 24, 25, 26, 27, 28, 29, 30, 35, 40, 45, 50, 55, 60, 65, 70, or 75 amino acids in length.

In some embodiments, the hinge domain is a hinge domain of a naturally occurring protein. Hinge domains of any protein known in the art to comprise a hinge domain are compatible for use in the chimeric receptors described herein. In some embodiments, the hinge domain is at least a portion of a hinge domain of a naturally occurring protein and confers flexibility to the chimeric receptor. In some embodiments, the hinge domain is of CD8α. In some embodiments, the hinge domain is a portion of the hinge domain of CD8α, e.g., a fragment containing at least 15 (e.g., 20, 25, 30, 35, or 40) consecutive amino acids of the hinge domain of CD8α.

Hinge domains of antibodies, such as an IgG, IgA, IgM, IgE, or IgD antibodies, are also compatible for use in the chimeric receptors described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

Non-naturally occurring peptides may also be used as hinge domains for the chimeric receptors described herein. In some embodiments, the hinge domain between the C-terminus of the extracellular ligand-binding domain of an Fc receptor and the N-terminus of the transmembrane domain is a peptide linker, such as a (GlyₓSer)ₙ linker, wherein x and n, independently can be an integer between 3 and 12, including 3, 4, 5, 6, 7, 8, 9, 10, 11, 12, or more. In some embodiments, the hinge domain is (Gly₄Ser)ₙ (SEQ ID NO: 76), wherein n can be an integer between 3 and 60, including 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more. In some embodiments, the hinge domain is (Gly₄Ser)₃ (SEQ ID NO: 77). In some embodiments, the hinge domain is (Gly₄Ser)₆ (SEQ ID NO: 78). In some embodiments, the hinge domain is (Gly₄Ser)₉ (SEQ ID NO: 79). In some embodiments, the hinge domain is (Gly₄Ser)₁₂ (SEQ ID NO: 80). In some embodiments, the hinge domain is (Gly₄Ser)₁₅ (SEQ ID NO: 81). In some embodiments, the hinge domain is (Gly₄Ser)₃₀ (SEQ ID NO: 82). In some embodiments, the hinge domain is (Gly₄Ser)₄₅ (SEQ ID NO: 83). In some embodiments, the hinge domain is (Gly₄Ser)₆₀ (SEQ ID NO: 84).

In other embodiments, the hinge domain is an extended recombinant polypeptide (XTEN), which is an unstructured polypeptide consisting of hydrophilic residues of varying lengths (e.g., 10-80 amino acid residues). Amino acid sequences of XTEN peptides will be evident to one of skill in the art and can be found, for example, in U.S. Pat. No. 8,673,860, which is herein incorporated by reference. In some embodiments, the hinge domain is an XTEN peptide and comprises 60 amino acids. In some embodiments, the hinge domain is an XTEN peptide and comprises 30 amino acids. In some embodiments, the hinge domain is an XTEN peptide and comprises 45 amino acids. In some embodiments, the hinge domain is an XTEN peptide and comprises 15 amino acids.

### F. Signal Peptide

In some embodiments, the chimeric receptor also comprises a signal peptide (also known as a signal sequence) at the N-terminus of the polypeptide. In general, signal sequences are peptide sequences that target a polypeptide to the desired site in a cell. In some embodiments, the signal sequence targets the chimeric receptor to the secretory pathway of the cell and will allow for integration and anchoring of the chimeric receptor into the lipid bilayer. Signal sequences including signal sequences of naturally occurring proteins or synthetic, non-naturally occurring signal sequences, that are compatible for use in the chimeric receptors described herein will be evident to one of skill in the art. In some embodiments, the signal sequence from CD8α. In some embodiments, the signal sequence is from CD28. In other embodiments, the signal sequence is from the murine kappa chain. In yet other embodiments, the signal sequence is from CD16.

### G. Examples of Chimeric Receptors

Tables 3-5 provide exemplary chimeric receptors described herein. This exemplary constructs have, from N-terminus to C-terminus in order, the signal sequence, the Fc binder (e.g., an extracellular domain of an Fc receptor), the hinge domain, and the transmembrane, while the positions of the co-stimulatory domain and the cytoplasmic signaling domain can be switched.

TABLE 3
Exemplary chimeric receptors.

| Exemplary | | | | | | |
|---|---|---|---|---|---|---|
| AA | | | | | | |
| Sequence | | Extracellular | | | Co- | Cytoplasmic |
| (SEQ ID | Signal | domain of | Hinge | Transmembrane | stimulatory | Signaling |
| NO) | Sequence | Fc receptor | domain | domain | domain | domain |
| 2 | CD8α | CD16A- | CD8α | 4-1BB (CD137) | 4-1BB | CD3ζ |
| | | V158 | | | (CD137) | |
| 3 | CD8α | CD16A- | CD8α | CD28 | 4-1BB | CD3ζ |
| | | V158 | | | (CD137) | |
| 4 | CD8α | CD16A- | CD8α | CD34 | 4-1BB | CD3ζ |
| | | V158 | | | (CD137) | |
| 5 | CD8α | CD16A- | CD8α | Designed | 4-1BB | CD3ζ |
| | | V158 | | hydrophobic | (CD137) | |
| | | | | TM domain | | |
| 6 | CD8α | CD32A | CD8α | CD8α | 4-1BB | CD3ζ |
| | | | | | (CD137) | |
| 7 | CD8α | CD16A- | CD8α | CD8α | CD28 | CD3ζ |
| | | V158 | | | | |
| 8 | CD8α | CD16A- | CD8α | CD8α | OX40 | CD3ζ |
| | | V158 | | | (CD134) | |
| 9 | CD8α | CD16A- | CD8α | CD8α | CD28 + | CD3ζ |
| | | V158 | | | 4-1BB | |
| 10 | CD8α | CD16A- | None | CD8α | 4-1BB | CD3ζ |
| | | V158 | | | (CD137) | |
| 11 | CD8α | CD16A- | XTEN | CD8α | 4-1BB | CD3ζ |
| | | V158 | | | (CD137) | |

TABLE 4
Exemplary chimeric receptors.

| Exemplary | | | | | | |
|---|---|---|---|---|---|---|
| AA | | | | | | |
| Sequence | | Extracellular | | | Co- | Cytoplasmic |
| (SEQ ID | Signal | domain of | Hinge | Transmembrane | stimulatory | Signaling |
| NO) | Sequence | Fc receptor | domain | domain | domain | domain |
| 12 | CD8α | CD16A- | CD8α | CD8α | CD28 LL | CD3ζ |
| | | V158 | | | to GG | |
| | | | | | mutant | |
| 13 | CD8α | CD16A- | CD8α | CD8α | CD28 LL | CD3ζ |
| | | V158 | | | to GG | |
| | | | | | mutant + | |
| | | | | | 4-1BB | |
| 14 | CD8α | CD16A- | CD8α | CD4 | 4-1BB | CD3ζ |
| | | V158 | | | (CD137) | |
| 15 | CD8α | CD16A- | CD8α | CD4 | CD28 LL | CD3ζ |
| | | V158 | | | to GG | |
| | | | | | mutant + | |
| | | | | | 4-1BB | |
| 16 | CD8α | CD16A- | CD8α | FcϵRIγ | 4-1BB | CD3ζ |
| | | V158 | | | (CD137) | |
| 17 | CD8α | CD16A- | CD8α | Designed | 4-1BB | CD3ζ |
| | | V158 | | hydrophobic TM | (CD137) | |
| | | | | domain, predicted | | |
| | | | | dimerization | | |

TABLE 5
Exemplary chimeric receptors.

| Exemplary | | | | | | |
|---|---|---|---|---|---|---|
| AA | | | | | | |
| Sequence | | Extracellular | | | Co- | |
| (SEQ ID | Signal | domain of | Hinge | Transmembrane | stimulatory | Signaling |
| NO) | Sequence | Fc receptor | domain | domain | domain | domain |
| 18 | CD8α | CD16A- | CD8α | CD8β | 4-1BB | CD3ζ |
| | | V158 | | | (CD137) | |
| 19 | CD8α | CD16A- | CD8α | C16α | 4-1BB | CD3ζ |
| | | V158 | | | (CD137) | |
| 20 | CD8α | CD16A- | CD8α | OX40 (CD134) | 4-1BB | CD3ζ |
| | | V158 | | | (CD137) | |
| 21 | CD8α | CD16A- | CD8α | CD3ζ | 4-1BB | CD3ζ |
| | | V158 | | | (CD137) | |
| 22 | CD8α | CD16A- | CD8α | CD3ϵ | 4-1BB | CD3ζ |
| | | V158 | | | (CD137) | |
| 23 | CD8α | CD16A- | CD8α | CD3γ | 4-1BB | CD3ζ |
| | | V158 | | | (CD137) | |
| 24 | CD8α | CD16A- | CD8α | CD3δ | 4-1BB | CD3ζ |
| | | V158 | | | (CD137) | |
| 25 | CD8α | CD16A- | CD8α | TCR-α | 4-1BB | CD3ζ |
| | | V158 | | | (CD137) | |
| 26 | CD8α | CD16A- | CD8α | CD32 | 4-1BB | CD3ζ |
| | | V158 | | | (CD137) | |
| 27 | CD8α | CD16A- | CD8α | CD64 | 4-1BB | CD3ζ |
| | | V158 | | | (CD137) | |
| 28 | CD8α | CD16A- | CD8α | VEGFR2 | 4-1BB | CD3ζ |
| | | V158 | | | (CD137) | |
| 29 | CD8α | CD16A- | CD8α | FAS | 4-1BB | CD3ζ |
| | | V158 | | | (CD137) | |
| 30 | CD8α | CD16A- | CD8α | FGFR2B | 4-1BB | CD3ζ |
| | | V158 | | | (CD137) | |
| 32 | CD8α | CD64A | CD8α | CD8α | 4-1BB | CD3ζ |
| | | | | | (CD137) | |
| 33 | CD8α | CD16A- | IgG1 (hinge- | CD8α | 4-1BB | CD3ζ |
| | | V158 | CH2—CH3) | | (CD137) | |
| 34 | CD8α | CD16A- | IgG1 (hinge- | CD8α | 4-1BB | CD3ζ |
| | | V158 | CH3) | | (CD137) | |
| 35 | CD8α | CD16A- | IgG1 (hinge) | CD8α | 4-1BB | CD3ζ |
| | | V158 | | | (CD137) | |
| 36 | CD8α | CD16A- | CD8-alpha | CD8α | 4-1BB | CD3ζ |
| | | V158 | fragment 1 | | (CD137) | |
| | | | (30 amino | | | |
| | | | acids) | | | |
| 37 | CD8α | CD16A- | CD8-alpha | CD8α | 4-1BB | CD3ζ |
| | | V158 | fragment 2 | | (CD137) | |
| | | | (15 amino | | | |
| | | | acids) | | | |
| 38 | CD8α | CD16A- | (Gly4Ser) × 3 | CD8α | 4-1BB | CD3ζ |
| | | V158 | (60 amino | | (CD137) | |
| | | | acids) | | | |
| 39 | CD8α | CD16A- | (Gly4Ser) × 6 | CD8α | 4-1BB | CD3ζ |
| | | V158 | (45 amino | | (CD137) | |
| | | | acids) | | | |
| 40 | CD8α | CD16A- | (Gly4Ser) × 9 | CD8α | 4-1BB | CD3ζ |
| | | V158 | (30 amino | | (CD137) | |
| | | | acids) | | | |
| 41 | CD8α | CD16A- | (Gly4Ser) × 12 | CD8α | 4-1BB | CD3ζ |
| | | V158 | (15 amino | | (CD137) | |
| | | | acids) | | | |
| 42 | CD8α | CD16A- | XTEN (60 | CD8α | 4-1BB | CD3ζ |
| | | V158 | amino acids) | | (CD137) | |
| 43 | CD8α | CD16A- | XTEN (30 | CD8α | 4-1BB | CD3ζ |
| | | V158 | amino acids) | | (CD137) | |
| 44 | CD8α | CD16A- | XTEN (15 | CD8α | 4-1BB | CD3ζ |
| | | V158 | amino acids) | | (CD137) | |
| 45 | CD28 | CD16A- | CD8α | CD8α | 4-1BB | CD3ζ |
| | | V158 | | | (CD137) | |
| 46 | Murine | CD16A- | CD8α | CD8α | 4-1BB | CD3ζ |
| | kappa | V158 | | | (CD137) | |
| | chain | | | | | |
| 47 | CD16 | CD16A- | CD8α | CD8α | 4-1BB | CD3ζ |
| | | V158 | | | (CD137) | |
| 48 | CD8α | CD16A- | CD8α | CD8α | ICOS | CD3ζ |
| | | V158 | | | | |
| 49 | CD8α | CD16A- | CD8α | CD8α | CD27 | CD3ζ |
| | | V158 | | | | |
| 50 | CD8α | CD16A- | CD8α | CD8α | GITR | CD3ζ |
| | | V158 | | | | |
| 51 | CD8α | CD16A- | CD8α | CD8α | HVEM | CD3ζ |
| | | V158 | | | | |
| 52 | CD8α | CD16A- | CD8α | CD8α | TIM1 | CD3ζ |
| | | V158 | | | | |
| 53 | CD8α | CD16A- | CD8α | CD8α | LFA1 | CD3ζ |
| | | V158 | | | (CD11a) | |
| 54 | CD8α | CD16A- | CD8α | CD8α | CD2 | CD3ζ |
| | | V158 | | | | |
| 55 | CD8α | CD16A- | CD8α | FcϵR1γ | 4-1BB | FcϵR1γ |
| | | V158 | | | (CD137) | |
| 56 | CD8α | CD16A- | CD8α | CD8α | 4-1BB | FcϵR1γ |
| | | V158 | | | (CD137) | |

Amino acid sequences of the example chimeric receptors are provided below (signal sequence italicized).

SEQ ID NO: 2:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIISFFLALTSTALLFLLFFLTLRFSVVKRGKRGRKKLLYIFK
QPFMRPVQTTQEEDGCSCRFPEEEEGGCELRVKFSRSADAPAYQQGQNQLYNELNLGRRE
EYDVLDKRRGRDPEMGGKPRRKNPQEGLYNELQKDKMAEAYSEIGMKGERRRGKGHDGLY
QGLSTATKDTYDALHMQALPPR
SEQ ID NO: 3:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDFWVLVVVGGVLACYSLLVTVAFIIFWVRSKKRGRKKLLYIFK
QPFMRPVQTTQEEDGCSCRFPEEEEGGCELRVKFSRSADAPAYQQGQNQLYNELNLGRRE
EYDVLDKRRGRDPEMGGKPRRKNPQEGLYNELQKDKMAEAYSEIGMKGERRRGKGHDGLY
QGLSTATKDTYDALHMQALPPR
SEQ ID NO: 4:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDLIALVTSGALLAVLGITGYFLMNRKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 5:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDLLAALLALLAALLALLAALLARSKKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 6:
MALPVTALLLPLALLLHAARPQAAAPPKAVLKLEPPWINVLQEDSVTLTCQGARSPESDS
IQWFHNGNLIPTHTQPSYRFKANNNDSGEYTCQTGQTSLSDPVHLTVLSEWLVLQTPHLE
FQEGETIMLRCHSWKDKPLVKVTFFQNGKSQKFSHLDPTFSIPQANHSHSGDYHCTGNIG
YTLFSSKPVTITVQVPSMGSSSPMGTTTPAPRPPTPAPTIASQPLSLRPEACRPAAGGAV
HTRGLDFACDIYIWAPLAGTCGVLLLSLVITLYCKRGRKKLLYIFKQPFMRPVQTTQEED
GCSCRFPEEEEGGCELRVKFSRSADAPAYQQGQNQLYNELNLGRREEYDVLDKRRGRDPE
MGGKPRRKNPQEGLYNELQKDKMAEAYSEIGMKGERRRGKGHDGLYQGLSTATKDTYDAL
HMQALPPR
SEQ ID NO: 7:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCRSKRSRLLHSDYMNMTPR
RPGPTRKHYQPYAPPRDFAAYRSRVKFSRSADAPAYQQGQNQLYNELNLGRREEYDVLDK
RRGRDPEMGGKPRRKNPQEGLYNELQKDKMAEAYSEIGMKGERRRGKGHDGLYQGLSTAT
KDTYDALHMQALPPR
SEQ ID NO: 8:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCALYLLRRDQRLPPDAHKP
PGGGSFRTPIQEEQADAHSTLAKIRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 9:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCRSKRSRLLHSDYMNMTPR
RPGPTRKHYQPYAPPRDFAAYRSKRGRKKLLYIFKQPFMRPVQTTQEEDGCSCRFPEEEE
GGCELRVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR
SEQ ID NO: 10:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQIYIWAPLAGTCGVLLLSLVITLYCKRG
RKKLLYIFKQPFMRPVQTTQEEDGCSCRFPEEEEGGCELRVKFSRSADAPAYQQGQNQLY
NELNLGRREEYDVLDKRRGRDPEMGGKPRRKNPQEGLYNELQKDKMAEAYSEIGMKGERR
RGKGHDGLYQGLSTATKDTYDALHMQALPPR
SEQ ID NO: 11:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQGGSPAGSPTSTEEGTSESATPESGPGT
STEPSEGSAPGSPAGSPTIYIWAPLAGTCGVLLLSLVITLYCKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 12:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCRSKRSRGGHSDYMNMTPR
RPGPTRKHYQPYAPPRDFAAYRSRVKFSRSADAPAYQQGQNQLYNELNLGRREEYDVLDK
RRGRDPEMGGKPRRKNPQEGLYNELQKDKMAEAYSEIGMKGERRRGKGHDGLYQGLSTAT
KDTYDALHMQALPPR
SEQ ID NO: 13:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCRSKRSRGGHSDYMNMTPR
RPGPTRKHYQPYAPPRDFAAYRSKRGRKKLLYIFKQPFMRPVQTTQEEDGCSCRFPEEEE
GGCELRVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR
SEQ ID NO: 14:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDMALIVLGGVAGLLLFIGLGIFFCVRKRGRKKLLYIFKQPFMR
PVQTTQEEDGCSCRFPEEEEGGCELRVKFSRSADAPAYQQGQNQLYNELNLGRREEYDVL
DKRRGRDPEMGGKPRRKNPQEGLYNELQKDKMAEAYSEIGMKGERRRGKGHDGLYQGLST
ATKDTYDALHMQALPPR
SEQ ID NO: 15:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDMALIVLGGVAGLLLFIGLGIFFCVRRSKRSRGGHSDYMNMTP
RRPGPTRKHYQPYAPPRDFAAYRSRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 16:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDLCYILDAILFLYGIVLTLLYCRLKKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 17:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDLLLILLGVLAGVLATLAALLARSKKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 18:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDITLGLLVAGVLVLLVSLGVAIHLCKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 19:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDVSFCLVMVLLFAVDTGLYFSVKTNKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 20:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDVAAILGLGLVLGLLGPLAILLALYKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 21:
MALEVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDLCYLLDGILFIYGVILTALFLRVKKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 22:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDVMSVATIVIVDICITGGLLLLVYYWSKNRKRGRKKLLYIFKQ
PFMRPVQTTQEEDGCSCRFPEEEEGGCELRVKFSRSADAPAYQQGQNQLYNELNLGRREE
YDVLDKRRGRDPEMGGKPRRKNPQEGLYNELQKDKMAEAYSEIGMKGERRRGKGHDGLYQ
GLSTATKDTYDALHMQALPPR
SEQ ID NO: 23:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDGFLFAEIVSIFVLAVGVYFIAGQDKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 24:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDGIIVTDVIATLLLALGVFCFAGHETKRGRKKLLYIFKQPFMR
PVQTTQEEDGCSCRFPEEEEGGCELRVKFSRSADAPAYQQGQNQLYNELNLGRREEYDVL
DKRRGRDPEMGGKPRRKNPQEGLYNELQKDKMAEAYSEIGMKGERRRGKGHDGLYQGLSI
ATKDTYDALHMQALPPR
SEQ ID NO: 25:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDVIGFRILLLKVAGFNLLMTLRLWKRGRKKLLYIFKQPFMRPV
QTTQEEDGCSCRFPEEEEGGCELRVKFSRSADAPAYQQGQNQLYNELNLGRREEYDVLDK
RRGRDPEMGGKPRRKNPQEGLYNELQKDKMAEAYSEIGMKGERRRGKGHDGLYQGLSTAT
KDTYDALHMQALPPR
SEQ ID NO: 26:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIIVAVVIATAVAAIVAAVVALIYCRKKRGRKKLLYIFKQPFM
RPVQTTQEEDGCSCRFPEEEEGGCELRVKFSRSADAPAYQQGQNQLYNELNLGRREEYDV
LDKRRGRDPEMGGKPRRKNPQEGLYNELQKDKMAEAYSEIGMKGERRRGKGHDGLYQGLS
TATKDTYDALHMQALPPR
SEQ ID NO: 27:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDVLFYLAVGIMFLVNTVLWVTIRKEKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 28:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIIILVGTAVIAMFFWLLLVIILRTKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 29:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDLGWLCLLLLPIPLIVWVKRKKRGRKKLLYIFKQPFMRPVQTT
QEEDGCSCRFPEEEEGGCELRVKFSRSADAPAYQQGQNQLYNELNLGRREEYDVLDKRRG
RDPEMGGKPRRKNPQEGLYNELQKDKMAEAYSEIGMKGERRRGKGHDGLYQGLSTATKDT
YDALHMQALPPR
SEQ ID NO: 30:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIAIYCIGVFLIACMVVTVILCRMKKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 31:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
FGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 32:
MALPVTALLLPLALLLHAARPQVDTTKAVITLQPPWVSVFQEETVTLHCEVLHLPGSSSI
QWFLNGTATQTSTPSYRITSASVNDSGEYRCQRGLSGRSDPIQLEIHRGWLLLQVSSRVF
TEGEPLALRCHAWKDKLVYNVLYYRNGKAFKFFHWNSNLTILKTNISHNGTYHCSGMGKH
RYTSAGISVTVKELFPAPVLNASVTSPLLEGNLVTLSCETKLLLQRPGLQLYFSFYMGSK
TLRGRNTSSEYQILTARREDSGLYWCEAATEDGNVLKRSPELELQVLGLQLPTPVWFHIY
IWAPLAGTCGVLLLSLVITLYCKRGRKKLLYIFKQPFMRPVQTTQEEDGCSCRFPEEEEG
GCELRVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR
SEQ ID NO: 33:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQEPKSCDKTHTCPPCPAPELLGGPSVFL
FPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTVDKSRWQQGNV
FSCSVMHEALHNHYTQKSLSLSPGKIYIWAPLAGTCGVLLLSLVITLYCKRGRKKLLYIF
KQPFMRPVQTTQEEDGCSCRFPEEEEGGCELRVKFSRSADAPAYQQGQNQLYNELNLGRR
EEYDVLDKRRGRDPEMGGKPRRKNPQEGLYNELQKDKMAEAYSEIGMKGERRRGKGHDGL
YQGLSTATKDTYDALHMQALPPR
SEQ ID NO: 34:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQEPKSCDKTHTCPGQPREPQVYTLPPSR
DELTKNQVSLTCLVKGFYPSDIAVEWESNGQPENNYKTTPPVLDSDGSFFLYSKLTVDKS
RWQQGNVFSCSVMHEALHNHYTQKSLSLSPGKIYIWAPLAGTCGVLLLSLVITLYCKRGR
KKLLYIFKQPFMRPVQTTQEEDGCSCRFPEEEEGGCELRVKFSRSADAPAYQQGQNQLYN
ELNLGRREEYDVLDKRRGRDPEMGGKPRRKNPQEGLYNELQKDKMAEAYSEIGMKGERRR
GKGHDGLYQGLSTATKDTYDALHMQALPPR
SEQ ID NO: 35:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQEPKSCDKTHTCPIYIWAPLAGTCGVLL
LSLVITLYCKRGRKKLLYIFKQPFMRPVQTTQEEDGCSCRFPEEEEGGCELRVKFSRSAD
APAYQQGQNQLYNELNLGRREEYDVLDKRRGRDPEMGGKPRRKNPQEGLYNELQKDKMAE
AYSEIGMKGERRRGKGHDGLYQGLSTATKDTYDALHMQALPPR
SEQ ID NO: 36:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAF
ACDIYIWAPLAGTCGVLLLSLVITLYCKRGRKKLLYIFKQPFMRPVQTTQEEDGCSCRFP
EEEEGGCELRVKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTYDALHMQALPP
R
SEQ ID NO: 37:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPFACDIYIWAPLAGTCG
VLLLSLVITLYCKRGRKKLLYIFKQPFMRPVQTTQEEDGCSCRFPEEEEGGCELRVKFSR
SADAPAYQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPR
SEQ ID NO: 38:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQGGGGSGGGGSGGGGSIYIWAPLAGTCG
VLLLSLVITLYCKRGRKKLLYIFKQPFMRPVQTTQEEDGCSCRFPEEEEGGCELRVKFSR
SADAPAYQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPR
SEQ ID NO: 39:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQGGGGSGGGGSGGGGSGGGGSGGGGSGG
GGSIYIWAPLAGTCGVLLLSLVITLYCKRGRKKLLYIFKQPFMRPVQTTQEEDGCSCRFP
EEEEGGCELRVKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTYDALHMQALPP
R
SEQ ID NO: 40:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQGGGGSGGGGSGGGGSGGGGSGGGGSGG
GGSGGGGSGGGGSGGGGSIYIWAPLAGTCGVLLLSLVITLYCKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 41:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQGGGGSGGGGSGGGGSGGGGSGGGGSGG
GGSGGGGSGGGGSGGGGSGGGGSGGGGSGGGGSIYIWAPLAGTCGVLLLSLVITLYCKRG
RKKLLYIFKQPFMRPVQTTQEEDGCSCRFPEEEEGGCELRVKFSRSADAPAYQQGQNQLY
NELNLGRREEYDVLDKRRGRDPEMGGKPRRKNPQEGLYNELQKDKMAEAYSEIGMKGERR
RGKGHDGLYQGLSTATKDTYDALHMQALPPR
SEQ ID NO: 42:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQGGSPAGSPTSTEEGTSESATPESGPGT
STEPSEGSAPGSPAGSPTSTEEGTSTEPSEGSAIYIWAPLAGTCGVLLLSLVITLYCKRG
RKKLLYIFKQPFMRPVQTTQEEDGCSCRFPEEEEGGCELRVKFSRSADAPAYQQGQNQLY
NELNLGRREEYDVLDKRRGRDPEMGGKPRRKNPQEGLYNELQKDKMAEAYSEIGMKGERR
RGKGHDGLYQGLSTATKDTYDALHMQALPPR
SEQ ID NO: 43:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQGGSPAGSPTSTEEGTSESATPESGPGT
STEIYIWAPLAGTCGVLLLSLVITLYCKRGRKKLLYIFKQPFMRPVQTTQEEDGCSCRFP
EEEEGGCELRVKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTYDALHMQALPP
R
SEQ ID NO: 44:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQGGSPAGSPTSTEEGTIYIWAPLAGTCG
VLLLSLVITLYCKRGRKKLLYIFKQPFMRPVQTTQEEDGCSCRFPEEEEGGCELRVKFSR
SADAPAYQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPR
SEQ ID NO: 45:
MLRLLLALNLFPSIQVTGGMRTEDLPKAVVFLEPQWYRVLEKDSVILKCQGAYSPEDNST
QWFHNESLISSQASSYFIDAATVDDSGEYRCQTNLSTLSDPVQLEVHIGWLLLQAPRWVF
KEEDPIHLRCHSWKNTALHKVTYLQNGKGRKYFHHNSDFYIPKATLKDSGSYFCRGLVGS
KNVSSETVNITITQGLAVSTISSFFPPGYQTTTPAPRPPTPAPTIASQPLSLRPEACRPA
AGGAVHTRGLDFACDIYIWAPLAGTCGVLLLSLVITLYCKRGRKKLLYIFKQPFMRPVQT
TQEEDGCSCRFPEEEEGGCELRVKFSRSADAPAYQQGQNQLYNELNLGRREEYDVLDKRR
GRDPEMGGKPRRKNPQEGLYNELQKDKMAEAYSEIGMKGERRRGKGHDGLYQGLSTATKD
TYDALHMQALPPR
SEQ ID NO: 46:
METDTLLLWVLLLWVPGSTGDGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTA
TKDTYDALHMQALPPR
SEQ ID NO: 47:
MWQLLLPTALLLLVSAGMRTEDLPKAVVFLEPQWYRVLEKDSVTLKCQGAYSPEDNSTQW
FHNESLISSQASSYFIDAATVDDSGEYRCQTNLSTLSDPVQLEVHIGWLLLQAPRWVFKE
EDPIHLRCHSWKNTALHKVTYLQNGKGRKYFHHNSDFYIPKATLKDSGSYFCRGLVGSKN
VSSETVNITITQGLAVSTISSFFPPGYQTTTPAPRPPTPAPTIASQPLSLRPEACRPAAG
GAVHTRGLDFACDIYIWAPLAGTCGVLLLSLVITLYCKRGRKKLLYIFKQPFMRPVQTTQ
EEDGCSCRFPEEEEGGCELRVKFSRSADAPAYQQGQNQLYNELNLGRREEYDVLDKRRGR
DPEMGGKPRRKNPQEGLYNELQKDKMAEAYSEIGMKGERRRGKGHDGLYQGLSTATKDTY
DALHMQALPPR
SEQ ID NO: 48:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCCWLTKKKYSSSVHDPNGE
YMFMRAVNTAKKSRLTDVTLRVKFSRSADAPAYQQGQNQLYNELNLGRREEYDVLDKRRG
RDPEMGGKPRRKNPQEGLYNELQKDKMAEAYSEIGMKGERRRGKGHDGLYQGLSTATKDT
YDALHMQALPPR
SEQ ID NO: 49:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCQRRKYRSNKGESPVEPAE
PCRYSCPREEEGSTIPIQEDYRKPEPACSPRVKFSRSADAPAYQQGQNQLYNELNLGRRE
EYDVLDKRRGRDPEMGGKPRRKNPQEGLYNELQKDKMAEAYSEIGMKGERRRGKGHDGLY
QGLSTATKDTYDALHMQALPPR
SEQ ID NO: 50:
MALEVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCQLGLHIWQLRSQCMWPRE
TQLLLEVPPSTEDARSCQFPEEERGERSAEEKGRLGDLWVRVKFSRSADAPAYQQGQNQL
YNELNLGRREEYDVLDKRRGRDPEMGGKPRRKNPQEGLYNELQKDKMAEAYSEIGMKGER
RRGKGHDGLYQGLSTATKDTYDALHMQALPPR
SEQ ID NO: 51:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCCVKRRKPRGDVVKVIVSV
QRKRQEAEGEATVIEALQAPPDVTTVAVEETIPSFTGRSPNHRVKFSRSADAPAYQQGQN
QLYNELNLGRREEYDVLDKRRGRDPEMGGKPRRKNPQEGLYNELQKDKMAEAYSEIGMKG
ERRRGKGHDGLYQGLSTATKDTYDALHMQALPPR
SEQ ID NO: 52:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCKKYFFKKEVQQLSVSFSS
LQIKALQNAVEKEVQAEDNIYIENSLYATDRVKFSRSADAPAYQQGQNQLYNELNLGRRE
EYDVLDKRRGRDPEMGGKPRRKNPQEGLYNELQKDKMAEAYSEIGMKGERRRGKGHDGLY
QGLSTATKDTYDALHMQALPPR
SEQ ID NO: 53:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCYKVGFFKRNLKEKMEAGR
GVPNGIPAEDSEQLASGQEAGDPGCLKPLHEKDSESGGGKDRVKFSRSADAPAYQQGQNQ
LYNELNLGRREEYDVLDKRRGRDPEMGGKPRRKNPQEGLYNELQKDKMAEAYSEIGMKGE
RRRGKGHDGLYQGLSTATKDTYDALHMQALPPR
SEQ ID NO: 54:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCKRKKQRSRRNDEELETRA
HRVATEERGRKPHQIPASTPQNPATSQHPPPPPGHRSQAPSHRPPPPGHRVQHQPQKRPP
APSGTQVHQQKGPPLPRPRVQPKPPHGAAENSLSPSSNRVKFSRSADAPAYQQGQNQLYN
ELNLGRREEYDVLDKRRGRDPEMGGKPRRKNPQEGLYNELQKDKMAEAYSEIGMKGERRR
GKGHDGLYQGLSTATKDTYDALHMQALPPR
SEQ ID NO: 55:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDPQLCYILDAILFLYGIVLTLLYCRLKIQVRKAAITSYEKSDG
VYTGLSTRNQETYETLKHEKPPQKRGRKKLLYIFKQPFMRPVQTTQEEDGCSCRFPEEEE
GGCEL
SEQ ID NO: 56:
MALPVTALLLPLALLLHAARPGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYFCRGL
VGSKNVSSETVNITITQGLAVSTISSFFPPGYQTTTPAPRPPTPAPTIASQPLSLRPEAC
RPAAGGAVHTRGLDFACDIYIWAPLAGTCGVLLLSLVITLYCKRGRKKLLYIFKQPFMRP
VQTTQEEDGCSCRFPEEEEGGCELRLKIQVRKAAITSYEKSDGVYTGLSTRNQETYETLK
HEKPPQ

In some embodiments, the chimeric receptor described herein may comprise one or more of an extracellular ligand-binding domain of CD16 (CD16F or CD16V, also known as F158 FCGR3A and V158 FCGR3A variant), hinge and transmembrane domains of CD8α, a co-stimulatory signaling domain of 4-1BB, and a cytoplasmic signaling domain of CD3ζ, e.g., the CD16F-BB-ζ and CD16V-BB-ζ disclosed herein. The amino acid sequences and exemplary coding nucleotide sequences of these components are provided in Table 6 below.

TABLE 6
Exemplary sequences

| SEQ ID | | |
|---|---|---|
| NO | SEQUENCE | DESCRIPTION |
| 1 | MALPVTALLLPLALLLHAARPGMRTEDLPKAVVFLEPQWYRVLEKDSVTLKCQGAYS | CD16V-BB-ζ |
| | PEDNSTQWFHNESLISSQASSYFIDAATVDDSGEYRCQTNLSTLSDPVQLEVHIGWL | |
| | LLQAPRWVFKEEDPIHLRCHSWKNTALHKVTYLQNGKGRKYFHHNSDFYIPKATLKD | |
| | SGSYFCRGLVGSKNVSSETVNITITQGLAVSTISSFFPPGYQTTTPAPRPPTPAPTI | |
| | ASQPLSLRPEACRPAAGGAVHTRGLDFACDIYIWAPLAGTCGVLLLSLVITLYCKRG | |
| | RKKLLYIFKQPFMRPVQTTQEEDGCSCRFPEEEEGGCELRVKFSRSADAPAYQQGQN | |
| | QLYNELNLGRREEYDVLDKRRGRDPEMGGKPRRKNPQEGLYNELQKDKMAEAYSEIG | |
| | MKGERRRGKGHDGLYQGLSTATKDTYDALHMQALPPR | |
| 57 | GMRTEDLPKAVVFLEPQWYRVLEKDSVTLKCQGAYSPEDNSTQWFHNESLISSQASS | V158 |
| | YFIDAATVDDSGEYRCQTNLSTLSDPVQLEVHIGWLLLQAPRWVFKEEDPIHLRCHS | FCGR3A |
| | WKNTALHKVTYLQNGKGRKYFHHNSDFYIPKATLKDSGSYFCRGLVGSKNVSSETVN | variant |
| | ITITQGLAVSTISSFFPPGYQ | |
| 58 | TTTPAPRPPTPAPTIASQPLSLRPEACRPAAGGAVHTRGLDFACDIYIWAPLAGTCG | hinge and |
| | VLLLSLVITLYC | transmembr |
| | | ane |
| | | domains of |
| | | CD8alpha |
| 59 | KRGRKKLLYIFKQPFMRPVQTTQEEDGCSCRFPEEEEGGCEL | 4-1BB |
| | | signaling |
| | | domain |
| 60 | RVKFSRSADAPAYQQGQNQLYNELNLGRREEYDVLDKRRGRDPEMGGKPRRKNPQEG | CD3 zeta |
| | LYNELQKDKMAEAYSEIGMKGERRRGKGHDGLYQGLSTATKDTYDALHMQALPPR | signaling |
| | | domain |
| 61 | MALPVTALLLPLALLLHAARP | signal |
| | | peptide of |
| | | CD8alpha |
| 62 | CTTCTGCAGGGGGCTTGTTGGGAGTAAAAATGTGTC | synthetic/ |
| | | primer |
| 63 | GACACATTTTTACTCCCAACAAGCCCCCTGCAGAAG | synthetic/ |
| | | primer |
| 64 | ATGGCCTTACCAGTGACCGCCTTGCTCCTGCCGCTGGCCTTGCTGCTCCACGCCGCC | CD16V-BB-ζ |
| | AGGCCGGGCATGCGGACTGAAGATCTCCCAAAGGCTGTGGTGTTCCTGGAGCCTCAA | |
| | TGGTACAGGGTGCTCGAGAAGGACAGTGTGACTCTGAAGTGCCAGGGAGCCTACTCC | |
| | CCTGAGGACAATTCCACACAGTGGTTTCACAATGAGAGCCTCATCTCAAGCCAGGCC | |
| | TCGAGCTACTTCATTGACGCTGCCACAGTCGACGACAGTGGAGAGTACAGGTGCCAG | |
| | ACAAACCTCTCCACCCTCAGTGACCCGGTGCAGCTAGAAGTCCATATCGGCTGGCTG | |
| | TTGCTCCAGGCCCCTCGGTGGGTGTTCAAGGAGGAAGACCCTATTCACCTGAGGTGT | |
| | CACAGCTGGAAGAACACTGCTCTGCATAAGGTCACATATTTACAGAATGGCAAAGGC | |
| | AGGAAGTATTTTCATCATAATTCTGACTTCTACATTCCAAAAGCCACACTCAAAGAC | |
| | AGCGGCTCCTACTTCTGCAGGGGGCTTGTTGGGAGTAAAAATGTGTCTTCAGAGACT | |
| | GTGAACATCACCATCACTCAAGGTTTGGCAGTGTCAACCATCTCATCATTCTTTCCA | |
| | CCTGGGTACCAAACCACGACGCCAGCGCCGCGACCACCAACACCGGCGCCCACCATC | |
| | GCGTCGCAGCCCCTGTCCCTGCGCCCAGAGGCGTGCCGGCCAGCGGCGGGGGGCGCA | |
| | GTGCACACGAGGGGGCTGGACTTCGCCTGTGATATCTACATCTGGGCGCCCTTGGCC | |
| | GGGACTTGTGGGGTCCTTCTCCTGTCACTGGTTATCACCCTTTACTGCAAACGGGGC | |
| | AGAAAGAAACTCCTGTATATATTCAAACAACCATTTATGAGACCAGTACAAACTACT | |
| | CAAGAGGAAGATGGCTGTAGCTGCCGATTTCCAGAAGAAGAAGAAGGAGGATGTGAA | |
| | CTGAGAGTGAAGTTCAGCAGGAGCGCAGACGCCCCCGCGTACCAGCAGGGCCAGAAC | |
| | CAGCTCTATAACGAGCTCAATCTAGGACGAAGAGAGGAGTACGATGTTTTGGACAAG | |
| | AGACGTGGCCGGGACCCTGAGATGGGGGGAAAGCCGAGAAGGAAGAACCCTCAGGAA | |
| | GGCCTGTACAATGAACTGCAGAAAGATAAGATGGCGGAGGCCTACAGTGAGATTGGG | |
| | ATGAAAGGCGAGCGCCGGAGGGGCAAGGGGCACGATGGCCTTTACCAGGGTCTCAGT | |
| | ACAGCCACCAAGGACACCTACGACGCCCTTCACATGCAGGCCCTGCCCCCTCGCTAA | |
| 65 | GGCATGCGGACTGAAGATCTCCCAAAGGCTGTGGTGTTCCTGGAGCCTCAATGGTAC | V158 |
| | AGGGTGCTCGAGAAGGACAGTGTGACTCTGAAGTGCCAGGGAGCCTACTCCCCTGAG | FCGR3A |
| | GACAATTCCACACAGTGGTTTCACAATGAGAGCCTCATCTCAAGCCAGGCCTCGAGC | variant |
| | TACTTCATTGACGCTGCCACAGTCGACGACAGTGGAGAGTACAGGTGCCAGACAAAC | |
| | CTCTCCACCCTCAGTGACCCGGTGCAGCTAGAAGTCCATATCGGCTGGCTGTTGCTC | |
| | CAGGCCCCTCGGTGGGTGTTCAAGGAGGAAGACCCTATTCACCTGAGGTGTCACAGC | |
| | TGGAAGAACACTGCTCTGCATAAGGTCACATATTTACAGAATGGCAAAGGCAGGAAG | |
| | TATTTTCATCATAATTCTGACTTCTACATTCCAAAAGCCACACTCAAAGACAGCGGC | |
| | TCCTACTTCTGCAGGGGGCTTGTTGGGAGTAAAAATGTGTCTTCAGAGACTGTGAAC | |
| | ATCACCATCACTCAAGGTTTGGCAGTGTCAACCATCTCATCATTCTTTCCACCTGGG | |
| | TACCAA | |
| 66 | ACCACGACGCCAGCGCCGCGACCACCAACACCGGCGCCCACCATCGCGTCGCAGCCC | hinge and |
| | CTGTCCCTGCGCCCAGAGGCGTGCCGGCCAGCGGCGGGGGGCGCAGTGCACACGAGG | transmembr |
| | GGGCTGGACTTCGCCTGTGATATCTACATCTGGGCGCCCTTGGCCGGGACTTGTGGG | ane |
| | GTCCTTCTCCTGTCACTGGTTATCACCCTTTACTGC | domains of |
| | | CD8alpha |
| 67 | AAACGGGGCAGAAAGAAACTCCTGTATATATTCAAACAACCATTTATGAGACCAGTA | 4-1BB |
| | CAAACTACTCAAGAGGAAGATGGCTGTAGCTGCCGATTTCCAGAAGAAGAAGAAGGA | signaling |
| | GGATGTGAACTG | domain |
| 68 | AGAGTGAAGTTCAGCAGGAGCGCAGACGCCCCCGCGTACCAGCAGGGCCAGAACCAG | CD3 zeta |
| | CTCTATAACGAGCTCAATCTAGGACGAAGAGAGGAGTACGATGTTTTGGACAAGAGA | signaling |
| | CGTGGCCGGGACCCTGAGATGGGGGGAAAGCCGAGAAGGAAGAACCCTCAGGAAGGC | domain |
| | CTGTACAATGAACTGCAGAAAGATAAGATGGCGGAGGCCTACAGTGAGATTGGGATG | |
| | AAAGGCGAGCGCCGGAGGGGCAAGGGGCACGATGGCCTTTACCAGGGTCTCAGTACA | |
| | GCCACCAAGGACACCTACGACGCCCTTCACATGCAGGCCCTGCCCCCTCGCTAA | |
| 69 | ATGGCCTTACCAGTGACCGCCTTGCTCCTGCCGCTGGCCTTGCTGCTCCACGCCGCC | signal |
| | AGGCCG | peptide of |
| | | CD8alpha |
| 31 | MALPVTALLLPLALLLHAARPGMRTEDLPKAVVFLEPQWYRVLEKDSVTLKCQGAYS | CD16F-BB-ζ |
| | PEDNSTQWFHNESLISSQASSYFIDAATVDDSGEYRCQTNLSTLSDPVQLEVHIGWL | |
| | LLQAPRWVFKEEDPIHLRCHSWKNTALHKVTYLQNGKGRKYFHHNSDFYIPKATLKD | |
| | SGSYFCRGLFGSKNVSSETVNITITQGLAVSTISSFFPPGYQTTTPAPRPPTPAPTI | |
| | ASQPLSLRPEACRPAAGGAVHTRGLDFACDIYIWAPLAGTCGVLLLSLVITLYCKRG | |
| | RKKLLYIFKQPFMRPVQTTQEEDGCSCRFPEEEEGGCELRVKFSRSADAPAYQQGQN | |
| | QLYNELNLGRREEYDVLDKRRGRDPEMGGKPRRKNPQEGLYNELQKDKMAEAYSEIG | |
| | MKGERRRGKGHDGLYQGLSTATKDTYDALHMQALPPR | |
| 70 | GMRTEDLPKAVVFLEPQWYRVLEKDSVTLKCQGAYSPEDNSTQWFHNESLISSQASS | F158 |
| | YFIDAATVDDSGEYRCQTNLSTLSDPVQLEVHIGWLLLQAPRWVFKEEDPIHLRCHS | FCGR3A |
| | WKNTALHKVTYLQNGKGRKYFHHNSDFYIPKATLKDSGSYFCRGLFGSKNVSSETVN | |
| | ITITQGLAVSTISSFFPPGYQ | |
| 71 | ATGGCCTTACCAGTGACCGCCTTGCTCCTGCCGCTGGCCTTGCTGCTCCACGCCGCC | CD16F-BB-ζ |
| | AGGCCGGGCATGCGGACTGAAGATCTCCCAAAGGCTGTGGTGTTCCTGGAGCCTCAA | |
| | TGGTACAGGGTGCTCGAGAAGGACAGTGTGACTCTGAAGTGCCAGGGAGCCTACTCC | |
| | CCTGAGGACAATTCCACACAGTGGTTTCACAATGAGAGCCTCATCTCAAGCCAGGCC | |
| | TCGAGCTACTTCATTGACGCTGCCACAGTCGACGACAGTGGAGAGTACAGGTGCCAG | |
| | ACAAACCTCTCCACCCTCAGTGACCCGGTGCAGCTAGAAGTCCATATCGGCTGGCTG | |
| | TTGCTCCAGGCCCCTCGGTGGGTGTTCAAGGAGGAAGACCCTATTCACCTGAGGTGT | |
| | CACAGCTGGAAGAACACTGCTCTGCATAAGGTCACATATTTACAGAATGGCAAAGGC | |
| | AGGAAGTATTTTCATCATAATTCTGACTTCTACATTCCAAAAGCCACACTCAAAGAC | |
| | AGCGGCTCCTACTTCTGCAGGGGGCTTTTTGGGAGTAAAAATGTGTCTTCAGAGACT | |
| | GTGAACATCACCATCACTCAAGGTTTGGCAGTGTCAACCATCTCATCATTCTTTCCA | |
| | CCTGGGTACCAAACCACGACGCCAGCGCCGCGACCACCAACACCGGCGCCCACCATC | |
| | GCGTCGCAGCCCCTGTCCCTGCGCCCAGAGGCGTGCCGGCCAGCGGCGGGGGGCGCA | |
| | GTGCACACGAGGGGGCTGGACTTCGCCTGTGATATCTACATCTGGGCGCCCTTGGCC | |
| | GGGACTTGTGGGGTCCTTCTCCTGTCACTGGTTATCACCCTTTACTGCAAACGGGGC | |
| | AGAAAGAAACTCCTGTATATATTCAAACAACCATTTATGAGACCAGTACAAACTACT | |
| | CAAGAGGAAGATGGCTGTAGCTGCCGATTTCCAGAAGAAGAAGAAGGAGGATGTGAA | |
| | CTGAGAGTGAAGTTCAGCAGGAGCGCAGACGCCCCCGCGTACCAGCAGGGCCAGAAC | |
| | CAGCTCTATAACGAGCTCAATCTAGGACGAAGAGAGGAGTACGATGTTTTGGACAAG | |
| | AGACGTGGCCGGGACCCTGAGATGGGGGGAAAGCCGAGAAGGAAGAACCCTCAGGAA | |
| | GGCCTGTACAATGAACTGCAGAAAGATAAGATGGCGGAGGCCTACAGTGAGATTGGG | |
| | ATGAAAGGCGAGCGCCGGAGGGGCAAGGGGCACGATGGCCTTTACCAGGGTCTCAGT | |
| | ACAGCCACCAAGGACACCTACGACGCCCTTCACATGCAGGCCCTGCCCCCTCGCTAA | |
| 72 | GGCATGCGGACTGAAGATCTCCCAAAGGCTGTGGTGTTCCTGGAGCCTCAATGGTAC | F158 |
| | AGGGTGCTCGAGAAGGACAGTGTGACTCTGAAGTGCCAGGGAGCCTACTCCCCTGAG | FCGR3A |
| | GACAATTCCACACAGTGGTTTCACAATGAGAGCCTCATCTCAAGCCAGGCCTCGAGC | variant |
| | TACTTCATTGACGCTGCCACAGTCGACGACAGTGGAGAGTACAGGTGCCAGACAAAC | |
| | CTCTCCACCCTCAGTGACCCGGTGCAGCTAGAAGTCCATATCGGCTGGCTGTTGCTC | |
| | CAGGCCCCTCGGTGGGTGTTCAAGGAGGAAGACCCTATTCACCTGAGGTGTCACAGC | |
| | TGGAAGAACACTGCTCTGCATAAGGTCACATATTTACAGAATGGCAAAGGCAGGAAG | |
| | TATTTTCATCATAATTCTGACTTCTACATTCCAAAAGCCACACTCAAAGACAGCGGC | |
| | TCCTACTTCTGCAGGGGGCTTTTTGGGAGTAAAAATGTGTCTTCAGAGACTGTGAAC | |
| | ATCACCATCACTCAAGGTTTGGCAGTGTCAACCATCTCATCATTCTTTCCACCTGGG | |
| | TACCAA | |

In some examples, the chimeric receptors described herein do not include all of the above-listed components. For example, the chimeric receptor described herein may not be any of the chimeric receptors listed in Table 7 below, or may not comprise one or more of the sequences listed in Table 6 above.

TABLE 7
Exemplary chimeric receptors.

| Exemplary | | | | | | |
|---|---|---|---|---|---|---|
| AA | | | | | | |
| Sequence | | Extracellular | | | Co- | |
| (SEQ ID | Signal | domain of | Hinge | Transmembrane | stimulatory | Signaling |
| NO) | Sequence | Fc receptor | domain | domain | domain | domain |
| 1 | CD8α | CD16A- | CD8α | CD8α | 4-1BB | CD3ζ |
| | | V158 | | | (CD137) | |
| 31 | CD8α | CD16A- | CD8α | CD8α | 4-1BB | CD3ζ |
| | | F158 | | | (CD137) | |

Like other chimeric receptors disclosed herein, expression of these exemplary chimeric receptors in immune cells such as T cells and NK cells, would confer ADCC capability to these cells and, therefore, would significantly augment the anti-tumor potential of monoclonal antibodies (as well as other anti-tumor molecules comprising the Fc portion, such as, e.g., a composite molecule constituted by a ligand (e.g., cytokine, immune cell receptor) binding a tumor surface receptor combined with the Fc-portion of an immunoglobulin or Fc-containing DNA or RNA), regardless of the targeted tumor-antigen.

Like other chimeric receptors described herein, these exemplary chimeric receptors are also universal chimeric receptors with potential for augmenting significantly the efficacy of antibody therapy against multiple tumors. As discussed in Example 1 below, when expressed in human T cells by retroviral transduction, the V158 receptor of the disclosure has a significantly higher affinity for human IgG including humanized antibodies such as the anti-CD20 antibody Rituximab as compared to an identical chimeric receptor containing the common F158 variant (also provided herein). Engagement of the chimeric receptor provokes T-cell activation, exocytosis of lytic granules and proliferation. CD16V-BB-ζ expressing T cells specifically kill lymphoma cell lines and primary chronic lymphocytic leukemia (CLL) cells in the presence of Rituximab at low effector: target ratio, even when CLL cultures are performed on bone marrow-derived mesenchymal cells. The anti-HER2 antibody Trastuzumab trigger chimeric receptor-mediated antibody-dependent cell cytotoxicity (ADCC) against breast and gastric cancer cells, and the anti-GD2 antibody hu14.18K322A against neuroblastoma and osteosarcoma cells. As further disclosed in the Examples section, T cells expressing the chimeric receptor and Rituximab in combination eradicated human lymphoma cells in immunodeficient mice, while T cells or antibody alone did not. To facilitate clinical translation of this technology, a method based on electroporation of the chimeric receptor mRNA was developed, leading to efficient and transient receptor expression without the use of viral vectors.

### H. Preparation of and Pharmaceutical Compositions Comprising Chimeric Receptors

Any of the chimeric receptors described herein can be prepared by a routine method, such as recombinant technology. Methods for preparing the chimeric receptors herein involve generation of a nucleic acid that encodes a polypeptide comprising each of the domains of the chimeric receptors, including the extracellular ligand-binding domain of an Fc receptor, the transmembrane domain, at least one co-stimulatory signaling domain, and the cytoplasmic signaling domain comprising an ITAM. In some embodiments, the nucleic acid also encodes a hinge domain between the extracellular ligand-binding domain of an Fc receptor and the transmembrane domain. The nucleic acid encoding the chimeric receptor may also encode a signal sequence. In some embodiments, the nucleic acid sequence encodes any one of the exemplary chimeric receptors provided by SEQ ID NO: 2-30 and 32-56.

Sequences of each of the components of the chimeric receptors may be obtained via routine technology, e.g., PCR amplification from any one of a variety of sources known in the art. In some embodiments, sequences of one or more of the components of the chimeric receptors are obtained from a human cell. Alternatively, the sequences of one or more components of the chimeric receptors can be synthesized. Sequences of each of the components (e.g., domains) can be joined directly or indirectly (e.g., using a nucleic acid sequence encoding a peptide linker) to form a nucleic acid sequence encoding the chimeric receptor, using methods such as PCR amplification or ligation. Alternatively, the nucleic acid encoding the chimeric receptor may be synthesized. In some embodiments, the nucleic acid is DNA. In other embodiments, the nucleic acid is RNA.

Any of the chimeric receptor proteins, nucleic acid encoding such, and expression vectors carrying such nucleic acid can be mixed with a pharmaceutically acceptable carrier to form a pharmaceutical composition, which is also within the scope of the present disclosure. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g. Remington: The Science and Practice of Pharmacy 20^{th} Ed. (2000) Lippincott Williams and Wilkins, Ed. K. E. Hoover.

The pharmaceutical compositions of the disclosure may also contain one or more additional active compounds as necessary for the particular indication being treated, preferably those with complementary activities that do not adversely affect each other. Non-limiting examples of possible additional active compounds include, e.g., IL2 as well as various agents listed in the discussion of combination treatments, below.

II. Immune Cells Expressing Chimeric receptors

Host cells expressing the chimeric receptors described herein provide a specific population of cells that can recognize target cells bound by Fc-containing therapeutic agents such as antibodies (e.g., therapeutic antibodies) or Fc-fusion proteins. Engagement of the extracellular ligand-binding domain of a chimeric receptor construct expressed on such host cells (e.g., immune cells) with the Fc portion of an antibody or an Fc-fusion protein transmits an activation signal to the co-stimulatory signaling domain(s) and the ITAM-containing cytoplasmic signaling domain of the chimeric receptor construct, which in turn activates cell proliferation and/or effector functions of the host cell, such as ADCC effects triggered by the host cells. The combination of co-stimulatory signaling domain(s) and the cytoplasmic signaling domain comprising an ITAM may allow for robust activation of multiple signaling pathways within the cell. In some embodiments, the host cells are immune cells, such as T cells, NK cells, macrophages, neutrophils, eosinophils, or any combination thereof. In some embodiments, the immune cells are T cells. In some embodiments, the immune cells are NK cells. In other embodiments, the immune cells can be established cell lines, for example, NK-92 cells.

The population of immune cells can be obtained from any source, such as peripheral blood mononuclear cells (PBMCs), bone marrow, tissues such as spleen, lymph node, thymus, or tumor tissue. A source suitable for obtaining the type of host cells desired would be evident to one of skill in the art. In some embodiments, the population of immune cells is derived from PBMCs. The type of host cells desired (e.g., immune cells such as T cells, NK cells, macrophages, neutrophils, eosinophils, or any combination thereof) may be expanded within the population of cells obtained by co-incubating the cells with stimulatory molecules, for example, anti-CD3 and anti-CD28 antibodies may be used for expansion of T cells.

To construct the immune cells that express any of the chimeric receptor constructs described herein, expression vectors for stable or transient expression of the chimeric receptor construct may be constructed via conventional methods as described herein and introduced into immune host cells. For example, nucleic acids encoding the chimeric receptors may be cloned into a suitable expression vector, such as a viral vector in operable linkage to a suitable promoter. The nucleic acids and the vector may be contacted, under suitable conditions, with a restriction enzyme to create complementary ends on each molecule that can pair with each other and be joined with a ligase. Alternatively, synthetic nucleic acid linkers can be ligated to the termini of the nucleic acid encoding the chimeric receptors. The synthetic linkers may contain nucleic acid sequences that correspond to a particular restriction site in the vector. The selection of expression vectors/plasmids/viral vectors would depend on the type of host cells for expression of the chimeric receptors, but should be suitable for integration and replication in eukaryotic cells.

A variety of promoters can be used for expression of the chimeric receptors described herein, including, without limitation, cytomegalovirus (CMV) intermediate early promoter, a viral LTR such as the Rous sarcoma virus LTR, HIV-LTR, HTLV-1 LTR, the simian virus 40 (SV40) early promoter, herpes simplex tk virus promoter. Additional promoters for expression of the chimeric receptors include any constitutively active promoter in an immune cell. Alternatively, any regulatable promoter may be used, such that its expression can be modulated within an immune cell.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression of the chimeric receptor.

In one specific embodiment, such vectors also include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art (see, for example, Suicide Gene Therapy: Methods and Reviews, Springer, Caroline J. (Cancer Research UK Centre for Cancer Therapeutics at the Institute of Cancer Research, Sutton, Surrey, UK), Humana Press, 2004) and include, for example, the Herpes Simplex Virus (HSV) thymidine kinase (TK) gene, cytosine daminase, purine nucleoside phosphorylase, nitroreductase and caspases such as caspase 8.

Suitable vectors and methods for producing vectors containing transgenes are well known and available in the art. Examples of the preparation of vectors for expression of chimeric receptors can be found, for example, in US2014/0106449, herein incorporated in its entirety by reference.

Any of the vectors comprising a nucleic acid sequence that encodes a chimeric receptor construct described herein is also within the scope of the present disclosure. Such a vector, or the sequence encoding a chimeric receptor contained therein, may be delivered into host cells such as host immune cells by a suitable method. Methods of delivering vectors to immune cells are well known in the art and may include DNA electroporation, RNA electroporation, transfection reagents such as liposomes, or viral transduction.

In some embodiments, the vectors for expression of the chimeric receptors are delivered to host cells by viral transduction. Exemplary viral methods for delivery include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors, and adeno-associated virus (AAV) vectors (see, e.g., PCT Publication Nos. WO 94/12649, WO 93/03769; WO 93/19191; WO 94/28938; WO 95/11984 and WO 95/00655). In some embodiments, the vectors for expression of the chimeric receptors are retroviruses. In some embodiments, the vectors for expression of the chimeric receptors are lentiviruses.

Examples of references describing retroviral transduction include Anderson et al., U.S. Pat. No. 5,399,346; Mann et al., Cell 33:153 (1983); Temin et al., U.S. Pat. No. 4,650,764; Temin et al., U.S. Pat. No. 4,980,289; Markowitz et al., J. Virol. 62:1120 (1988); Temin et al., U.S. Pat. No. 5,124,263; International Patent Publication No. WO 95/07358, published Mar. 16, 1995, by Dougherty et al.; and Kuo et al., Blood 82:845 (1993). International Patent Publication No. WO 95/07358 describes high efficiency transduction of primary B lymphocytes. See also the Examples section, below, for examples of specific techniques for retroviral transduction and mRNA electroporation which can be used.

In examples in which the vectors encoding chimeric receptors are introduced to the host cells using a viral vector, viral particles that are capable of infecting the immune cells and carry the vector may be produced by any method known in the art and can be found, for example in PCT Application No. WO 1991/002805A2, WO 1998/009271 A1, and U.S. Patent 6,194,191. The viral particles are harvested from the cell culture supernatant and may be isolated and/or purified prior to contacting the viral particles with the immune cells.

In some embodiments, RNA molecules encoding any of the chimeric receptors as described herein may be prepared by a conventional method (e.g., in vitro transcription) and then introduced into suitable host cells, e.g., those described herein, via known method, e.g., Rabinovich et al., Human Gene Therapy 17:1027-1035. As demonstrated in the Examples below, mRNA electroporation results in effective expression of the chimeric receptors of the disclosure in T lymphocytes.

Following introduction into the host cells a vector encoding any of the chimeric receptors provided herein, or the nucleic acid encoding a chimeric vector (e.g., an RNA molecule), the cells are cultured under conditions that allow for expression of the chimeric receptor. In examples in which the nucleic acid encoding the chimeric receptor is regulated by a regulatable promoter, the host cells are cultured in conditions wherein the regulatable promoter is activated. In some embodiments, the promoter is an inducible promoter and the immune cells are cultured in the presence of the inducing molecule or in conditions in which the inducing molecule is produced. Determining whether the chimeric receptor is expressed will be evident to one of skill in the art and may be assessed by any known method, for example, detection of the chimeric receptor-encoding mRNA by quantitative reverse transcriptase PCR (qRT-PCR) or detection of the chimeric receptor protein by methods including Western blotting, fluorescence microscopy, and flow cytometry. Alternatively, expression of the chimeric receptor may take place in vivo after the immune cells are administered to a subject.

Alternatively, expression of a chimeric receptor construct in any of the immune cells disclosed herein can be achieved by introducing RNA molecules encoding the chimeric receptor constructs. Such RNA molecules can be prepared by in vitro transcription or by chemical synthesis. The RNA molecules can then introduced into suitable host cells such as immune cells (e.g., T cells, NK cells, macrophages, neutrophils, eosinophils, or any combination thereof) by, e.g., electroporation. For example, RNA molecules can be synthesized and introduced into host immune cells following the methods described in Rabinovich et al., Human Gene Therapy, 17:1027-1035 and WO WO2013/040557.

Methods for preparing host cells expressing any of the chimeric receptors described herein may also comprise activating the host cells ex vivo. Activating a host cell means stimulating a host cell into an activate state in which the cell may be able to perform effector functions (e.g., ADCC). Methods of activating a host cell will depend on the type of host cell used for expression of the chimeric receptors. For example, T cells may be activated ex vivo in the presence of one or more molecule such as an anti-CD3 antibody, an anti-CD28 antibody, IL-2, or phytohemoagglutinin. In other examples, NK cells may be activated ex vivo in the presence of one or molecules such as a 4-1BB ligand, an anti-4-1BB antibody, IL-15, an anti-IL-15 receptor antibody, IL-2, IL12, IL-21, and K562 cells. In some embodiments, the host cells expressing any of the chimeric receptors described herein are activated ex vivo prior to administration to a subject. Determining whether a host cell is activated will be evident to one of skill in the art and may include assessing expression of one or more cell surface markers associated with cell activation, expression or secretion of cytokines, and cell morphology.

The methods of preparing host cells expressing any of the chimeric receptors described herein may comprise expanding the host cells ex vivo. Expanding host cells may involve any method that results in an increase in the number of cells expressing chimeric receptors, for example, allowing the host cells to proliferate or stimulating the host cells to proliferate. Methods for stimulating expansion of host cells will depend on the type of host cell used for expression of the chimeric receptors and will be evident to one of skill in the art. In some embodiments, the host cells expressing any of the chimeric receptors described herein are expanded ex vivo prior to administration to a subject.

In some embodiments, the host cells expressing the chimeric receptors are expanded and activated ex vivo prior to administration of the cells to the subject. Host cell activation and expansion may be used to allow integration of a viral vector into the genome and expression of the gene encoding a chimeric receptor as described herein. If mRNA electroporation is used, no activation and/or expansion may be required, although electroporation may be more effective when performed on activated cells. In some instances, a chimeric receptor is transiently expressed in a suitable host cell (e.g., for 3-5 days). Transient expression may be advantageous if there is a potential toxicity and should be helpful in initial phases of clinical testing for possible side effects.

### IV. Application of Immune Cells Expressing A Chimeric Receptor in Immunotherapy

The exemplary chimeric receptors of the present disclosure confer antibody-dependent cell cytotoxicity (ADCC) capacity to T lymphocytes and enhance ADCC in NK cells. When the receptor is engaged by an antibody (or another anti-tumor molecule comprising the Fc portion) bound to tumor cells, it triggers T-cell activation, sustained proliferation and specific cytotoxicity against cancer cells targeted by the antibody (or such other anti-tumor molecule comprising the Fc portion). As disclosed in the Examples section, below, T lymphocytes comprising the chimeric receptors of the disclosure were highly cytotoxic against a wide range of tumor cell types, including B-cell lymphoma, breast and gastric cancer, neuroblastoma and osteosarcoma, as well as primary chronic lymphocytic leukemia (CLL). Cytotoxicity was entirely dependent on the presence of a specific antibody bound to target cells: soluble antibodies did not induce exocytosis of cytolytic granules and did not provoke non-specific cytotoxicity.

The degree of affinity of CD16 for the Fc portion of Ig is a critical determinant of ADCC and thus to clinical responses to antibody immunotherapy. The CD16 with the V158 polymorphism which has a high binding affinity for Ig and mediates superior ADCC was selected as an example. Although the F158 receptor has lower potency than the V158 receptor in induction of T cell proliferation and ADCC, the F158 receptor may have lower in vivo toxicity than the V158 receptor making it useful in some clinical contexts.

The chimeric receptors of the present disclosure facilitate T-cell therapy by allowing one single receptor to be used for multiple cancer cell types. It also allows the targeting of multiple antigens simultaneously, a strategy that may ultimately be advantageous given immunoescape mechanism exploited by tumors. Grupp et al., N Engl J Med. 2013. Antibody-directed cytotoxicity could be stopped whenever required by simple withdrawal of antibody administration. Because the T cells expressing the chimeric receptors of the disclosure are only activated by antibody bound to target cells, unbound immunoglobulin should not exert any stimulation on the infused T cells. Clinical safety can be further enhanced by using mRNA electroporation to express the chimeric receptors transiently, to limit any potential autoimmune reactivity.

The results disclosed in the Examples section, below, suggest that the infusion of autologous T cells, activated and expanded ex vivo and re-infused after genetic modification with the chimeric receptors of the disclosure should significantly boost ADCC. Because the combined CD3/4-1BB signaling also causes T-cell proliferation, there should be an accumulation of activated T cells at the tumor site which may further potentiate their activity.

Thus, in one embodiment, the disclosure provides a method for enhancing efficacy of an antibody-based immunotherapy of a cancer in a subject in need thereof, which subject is being treated with an antibody which can bind to cancer cells and has a humanized Fc portion which can bind to human CD16, said method comprising introducing into the subject a therapeutically effective amount of T lymphocytes or NK cells, which T lymphocytes or NK cells comprise a chimeric receptor of the disclosure.

### A. Enhancing Immune Therapy Efficacy

Host cells (e.g., immune cells) expressing chimeric receptors (the encoding nucleic acids or vectors comprising such) described herein are useful for enhancing ADCC in a subject and/or for enhancing the efficacy of an antibody-based immunotherapy. In some embodiments, the subject is a mammal, such as a human, monkey, mouse, rabbit, or domestic mammal. In some embodiments, the subject is a human. In some embodiments, the subject is a human cancer patient. In some embodiments, the subject has been treated or is being treated with any of the therapeutic antibodies described herein.

The immune cells can be mixed with a pharmaceutically acceptable carrier to form a pharmaceutical composition, which is also within the scope of the present disclosure.

To perform the methods described herein, an effective amount of the immune cells expressing any of the chimeric receptor constructs described herein can be administered into a subject in need of the treatment. The immune cells may be autologous to the subject, i.e., the immune cells are obtained from the subject in need of the treatment, genetically engineered for expression of the chimeric receptor constructs, and then administered to the same subject. Administration of autologous cells to a subject may result in reduced rejection of the host cells as compared to administration of non-autologous cells. Alternatively, the host cells are allogeneic cells, i.e., the cells are obtained from a first subject, genetically engineered for expression of the chimeric receptor construct, and administered to a second subject that is different from the first subject but of the same species. For example, allogeneic immune cells may be derived from a human donor and administered to a human recipient who is different from the donor.

In some embodiments, the immune cells are administered to a subject in an amount effective in enhancing ADCC activity by least 20%, e.g., 50%, 80%, 100%, 2-fold, 5-fold, 10-fold, 20-fold, 50-fold, 100-fold or more.

In some embodiments, the immune cells are co-used with a therapeutic Fc-containing therapeutic agent (e.g., an antibody or Fc fusion molecule such as Fc fusion protein) so as to enhance the efficacy of the anti-based immunotherapy. Antibody-based immunotherapy is used to treat, alleviate, or reduce the symptoms of any disease or disorder for which the immunotherapy is considered useful in a subject. In such therapy, a therapeutic antibody may bind to a cell surface antigen that is differentially expressed on cancer cells (i.e., not expressed on non-cancer cells or expressed at a lower level on non-cancer cells). Examples of antigens or target molecules that are bound by therapeutic antibodies and indicate that the cell expressing the antigen or target molecule should be subjected to ADCC include, without limitation, CD17/L1-CAM, CD19, CD20, CD22, CD30, CD33, CD37, CD52, CD56, CD70, CD79b, CD138, CEA, DS6, EGFR, EGFRvIII, ENPP3, FR, GD2, GPNMB, HER2, IL-13Rα2, Mesothelin, MUC1, MUC16, Nectin-4, PSMA, and SCL44A4.

The efficacy of an antibody-based immunotherapy may be assessed by any method known in the art and would be evident to a skilled medical professional. For example, the efficacy of the antibody-based immunotherapy may be assessed by survival of the subject or tumor or cancer burden in the subject or tissue or sample thereof. In some embodiments, the immune cells are administered to a subject in need of the treatment in an amount effective in enhancing the efficacy of an antibody-based immunotherapy by at least 20%, e.g., 50%, 80%, 100%, 2-fold, 5-fold, 10-fold, 20-fold, 50-fold, 100-fold or more, as compared to the efficacy in the absence of the immune cells.

In any of the methods described herein, the immune cells such as the T lymphocytes or NK cells, can be autologous cells isolated from the subject who is subject to the treatment. In one specific embodiment, prior to re-introduction into the subject, the autologous immune cells (e.g., T lymphocytes or NK cells) are activated and/or expanded ex vivo. In another embodiment, the immune cells (e.g., T lymphocytes or NK cells) are allogeneic cells.

In one specific embodiment, the T lymphocytes are allogeneic T lymphocytes in which the expression of the endogenous T cell receptor has been inhibited or eliminated. In one specific embodiment, prior to introduction into the subject, the allogeneic T lymphocytes are activated and/or expanded ex vivo. T lymphocytes can be activated by any method known in the art, e.g., in the presence of anti-CD3/CD28, IL-2, and/or phytohemoagglutinin.

NK cells can be activated by any method known in the art, e.g., in the presence of one or more agents selected from the group consisting of CD137 ligand protein, CD137 antibody, IL-15 protein, IL-15 receptor antibody, IL-2 protein, IL-12 protein, IL-21 protein, and K562 cell line. See, e.g., U.S. Pat. Nos. 7,435,596 and 8,026,097 for the description of useful methods for expanding NK cells. For example, NK cells used in the methods of the disclosure may be preferentially expanded by exposure to cells that lack or poorly express major histocompatibility complex I and/or II molecules and which have been genetically modified to express membrane bound IL-15 and 4-1BB ligand (CDI37L). Such cell lines include, but are not necessarily limited to, K562 [ATCC, CCL 243; Lozzio et al., Blood 45(3): 321-334 (1975); Klein et al., Int. J. Cancer 18: 421-431 (1976)], and the Wilms tumor cell line HFWT (Fehniger et al., Int Rev Immunol 20(3-4):503-534 (2001); Harada H, et al., Exp Hematol 32(7):614-621 (2004)), the uterine endometrium tumor cell line HHUA, the melanoma cell line HMV-II, the hepatoblastoma cell line HuH-6, the lung small cell carcinoma cell lines Lu-130 and Lu-134-A, the neuroblastoma cell lines NB 19 and N1369, the embryonal carcinoma cell line from testis NEC 14, the cervix carcinoma cell line TCO-2, and the bone marrow-metastasized neuroblastoma cell line TNB 1 [Harada, et al., Jpn. J. Cancer Res 93: 313-319 (2002)]. Preferably the cell line used lacks or poorly expresses both MHC I and II molecules, such as the K562 and HFWT cell lines. A solid support may be used instead of a cell line. Such support should preferably have attached on its surface at least one molecule capable of binding to NK cells and inducing a primary activation event and/or a proliferative response or capable of binding a molecule having such an affect thereby acting as a scaffold. The support may have attached to its surface the CD137 ligand protein, a CD137 antibody, the IL-15 protein or an IL-15 receptor antibody. Preferably, the support will have IL-15 receptor antibody and CD137 antibody bound on its surface.

In one embodiment of the above methods, introduction (or re-introduction) of T lymphocytes or NK cells to the subject is followed by administering to the subject a therapeutically effective amount of IL-2.

The chimeric receptors of the disclosure may be used for treatment of any cancer, including, without limitation, carcinomas, lymphomas, sarcomas, blastomas, and leukemias, for which a specific antibody with an Fc portion that binds to the Fc binder in the chimeric receptor exists or is capable of being generated. Specific non-limiting examples of cancers, which can be treated by the chimeric receptors of the disclosure include, e.g., cancers of B-cell origin (e.g., B-lineage acute lymphoblastic leukemia, B-cell chronic lymphocytic leukemia and B-cell non-Hodgkin's lymphoma), breast cancer, gastric cancer, neuroblastoma, and osteosarcoma.

To practice the method disclosed herein, an effective amount of the immune cells expressing chimeric receptors, Fc-containing therapeutic agents (e.g., Fc-containing therapeutic proteins such as Fc fusion proteins and therapeutic antibodies), or compositions thereof can be administered to a subject (e.g., a human cancer patient) in need of the treatment via a suitable route, such as intravenous administration. Any of the immune cells expressing chimeric receptors, Fc-containing therapeutic agents, or compositions thereof may be administered to a subject in an effective amount. As used herein, an effective amount refers to the amount of the respective agent (e.g., the host cells expressing chimeric receptors, Fc-containing therapeutic agents, or compositions thereof) that upon administration confers a therapeutic effect on the subject. Determination of whether an amount of the cells or compositions described herein achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human.

In some embodiments, the subject is a human cancer patient. For example, the subject can be a human patient suffering from carcinoma, lymphoma, sarcoma, blastoma, or leukemia. Examples of cancers for which administration of the cells and compositions disclosed herein may be suitable include, for example, lymphoma, breast cancer, gastric cancer, neuroblastoma, osteosarcoma, lung cancer, skin cancer, prostate cancer, colon cancer, renal cell carcinoma, ovarian cancer, rhabdomyosarcoma, leukemia, mesothelioma, pancreatic cancer, head and neck cancer, retinoblastoma, glioma, glioblastoma, and thyroid cancer.

In accordance with the present disclosure, patients can be treated by infusing therapeutically effective doses of immune cells such as T lymphocytes or NK cells comprising a chimeric receptor of the disclosure in the range of about 10⁵ to 10¹⁰ or more cells per kilogram of body weight (cells/Kg). The infusion can be repeated as often and as many times as the patient can tolerate until the desired response is achieved. The appropriate infusion dose and schedule will vary from patient to patient, but can be determined by the treating physician for a particular patient. Typically, initial doses of approximately 10⁶ cells/Kg will be infused, escalating to 10⁸ or more cells/Kg. IL-2 can be co-administered to expand infused cells post-infusion. The amount of IL-2 can about 1-5×10⁶ international units per square meter of body surface.

In some embodiments, the immune cells expressing any of the chimeric receptors disclosed herein are administered to a subject who has been treated or is being treated with an Fc-containing therapeutic agent (e.g., an Fc-fusion protein or a therapeutic antibody). The immune cells expressing any one of the chimeric receptors disclosed herein may be co-administered with an Fc-containing therapeutic agent. For example, the immune cells may be administered to a human subject simultaneously with a therapeutic antibody. Alternatively, the immune cells may be administered to a human subject during the course of an antibody-based immunotherapy. In some examples, the immune cells and an therapeutic antibody can be administered to a human subject at least 4 hours apart, e.g., at least 12 hours apart, at least 1 day apart, at least 3 days apart, at least one week apart, at least two weeks apart, or at least one month apart.

Examples of therapeutic Fc-containing therapeutic protein include, without limitation, Adalimumab, Ado-Trastuzumab emtansine, Alemtuzumab, Basiliximab, Bevacizumab, Belimumab, Brentuximab, Canakinumab, Cetuximab, Daclizumab, Denosumab, Dinoutuzimab, Eculizumab, Efalizumab, Epratuzumab, Gemtuzumab, Golimumab, Infliximab, Ipilimumab, Labetuzumab, Natalizumab, Obinutuzumab, Ofatumumab, Omalizumab, Palivizumab, Panitumumab, Pertuzumab, Ramucirumab, Ritutimab, Tocilizumab, Tratuzumab, Ustekinumab, and Vedolizumab.

The appropriate dosage of the Fc-containing therapeutic agent used will depend on the type of cancer to be treated, the severity and course of the disease, previous therapy, the patient's clinical history and response to the antibody, and the discretion of the attending physician. The antibody can be administered to the patient at one time or over a series of treatments. The progress of the therapy of the disclosure can be easily monitored by conventional techniques and assays.

The administration of Fc-containing therapeutic agent can be performed by any suitable route, including systemic administration as well as administration directly to the site of the disease (e.g., to primary tumor).

### B. Combination Treatments

The compositions and methods described in the present disclosure may be utilized in conjunction with other types of therapy for cancer, such as chemotherapy, surgery, radiation, gene therapy, and so forth. Such therapies can be administered simultaneously or sequentially (in any order) with the immunotherapy according to the present disclosure.

When co-administered with an additional therapeutic agent, suitable therapeutically effective dosages for each agent may be lowered due to the additive action or synergy.

The treatments of the disclosure can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 41BB, OX40, etc.).

Non-limiting examples of other therapeutic agents useful for combination with the immunotherapy of the disclosure include: (i) anti-angiogenic agents (e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000)); (ii) a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof; and (iii) chemotherapeutic compounds such as, e.g., pyrimidine analogs (5-fluorouracil, floxuridine, capecitabine, gemcitabine and cytarabine), purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

For examples of additional useful agents see also Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

### V. Kits for Therapeutic Use

The present disclosure also provides kits for use of the chimeric receptors in enhancing antibody-dependent cell-mediated cytotoxicity and enhancing an antibody-based immunotherapy. Such kits may include one or more containers comprising a first pharmaceutical composition that comprises any nucleic acid or host cells (e.g., immune cells such as those described herein), and a pharmaceutically acceptable carrier, and a second pharmaceutical composition that comprises a therapeutic antibody and a pharmaceutically acceptable carrier.

In some embodiments, the kit can comprise instructions for use in any of the methods described herein. The included instructions can comprise a description of administration of the first and second pharmaceutical compositions to a subject to achieve the intended activity, e.g., enhancing ADCC activity, and/or enhancing the efficacy of an antibody-based immunotherapy, in a subject. The kit may further comprise a description of selecting a subject suitable for treatment based on identifying whether the subject is in need of the treatment. In some embodiments, the instructions comprise a description of administering the first and second pharmaceutical compositions to a subject who is in need of the treatment.

The instructions relating to the use of the chimeric receptors and the first and second pharmaceutical compositions described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. At least one active agent in the pharmaceutical composition is a chimeric receptor as described herein.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D.N. Glover ed. 1985); Nucleic Acid Hybridization (B. D. Hames & S. J. Higgins eds.(1985>>; Transcription and Translation (B. D. Hames & S. J. Higgins, eds. (1984>>; Animal Cell Culture (R. I. Freshney, ed. (1986>>; Immobilized Cells and Enzymes (IRL Press, (1986>>; and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1

### T Lymphocytes Expressing a CD16 Signaling Receptor Exert Antibody Dependent Cancer Cell Killing

### Materials and Methods

### Cells

The human B-lineage lymphoma cell lines Daudi and Ramos, the T-cell acute lymphoblastic leukemia cell line Jurkat, and the neuroblastoma cell lines CHLA-255, NB1691 and SK-N-SH were available at St. Jude Children's Research Hospital. The breast carcinoma cell lines MCF-7 (ATCC HTB-22) and SK-BR-3 (ATCC HTB-30), and the osteosarcoma cell line U-2 OS (ATCC HTB-96) were obtained from the American Type Culture Collection (ATCC; Rockville, MD); the gastric carcinoma cell line MKN7 was from National Institute of Biomedical Innovation (Osaka, Japan). Daudi, CHLA-255, NB1691, SK-N-SH, SK-BR-3, MCF-7, U-2 OS and MKN7 were also transduced with a murine stem cell virus (MSCV)-internal ribosome entry site (IRES)-green fluorescent protein (GFP) retroviral vector containing the firefly luciferase gene.³⁴ Transduced cells were selected for their expression of GFP with a FACSAria cell sorter (BD Biosciences, San Jose, Calif.). Peripheral blood or bone marrow samples from newly diagnosed and untreated patients with B-chronic lymphocytic leukemia (CLL) were obtained following informed consent and approval from the Domain Specific Ethics Board governing Singapore's National University Hospital.

Peripheral blood samples were obtained from de-identified by-products of platelet donations from healthy adult donors. Mononuclear cells were enriched by centrifugation on Accu-Prep Human Lymphocytes Cell Separation Media (Accurate Chemical & Scientific Corp., Westbury, N.Y.), and cultured with anti-CD3/CD28 beads (Invitrogen, Carlsbad, Calif.) in RPMI-1640 with 10% fetal bovine serum (FBS), antibiotics, 100 IU interleukin (IL)-2 (Roche, Mannheim, Germany) for 3days. On day 4, T cells were purified by negative selection with a mixture of CD14, CD16, CD19, CD36, CD56, CD123 and CD235a antibodies and magnetic beads (Pan T Cell Isolation Kit II; Miltenyi Biotec, Bergisch Gladbach, Germany) (purity, >98%). Purified T cells were maintained in the above medium, with the addition of 100 IU IL-2 every other day.

Plasmids, virus production and gene transduction

The pMSCV-IRES-GFP, pEQ-PAM3(-E), and pRDF were obtained from the St. Jude Children's Research Hospital Vector Development and Production Shared Resource (Memphis, Tenn.).¹° The FCRG3A cDNA was obtained from Origene (Rockville, Md.) and its V158F variant was generated using site-directed mutagenesis by PCR using primers “F” CTTCTGCAGGGGGCTTGTTGGGAGTAAAAATGTGTC (SEQ ID NO: 73) and “R” GACACATTTTTACTCCCAACAAGCCCCCTGCAGAAG (SEQ ID NO: 74). The polynucleotides encoding CD8α hinge and transmembrane domain (SEQ ID NO: 66), and the intracellular domains of 4-1BB (SEQ ID NO: 67) and CD3ζ (SEQ ID NO: 68) were subcloned from an anti-CD19-41BB-CD3ζ cDNA previously made. Imai et al., 2004. These molecules were assembled using splicing by overlapping extension by PCR. The constructs (“CD16F-BB-ζ” and “CD16V-BB-ζ”) and the expression cassette were subcloned into EcoRI and MLu1 sites of the MSCV-IRES-GFP vector.

To generate RD114-pseudotyped retrovirus, fuGENE 6 or X-tremeGENE 9 (Roche, Indianapolis, Ind.) was used to transfect 3×10⁶ 293T cells with 3.5 μg of cDNA encoding CD16V-BB-ζ, 3.5 μg of pEQ-PAM3(-E), and 3 μg of pRDF. Imai et al., 2004. After replacing the medium with RPMI-1640 with 10% FBS at 24 hours, the medium containing retrovirus was harvested after 48-96 hours and added to RetroNectin (TakaRa, Otsu, Japan)-coated polypropylene tubes, which were centrifugated at 1400 g for 10 min and incubated at 37° C. for 6 hours. After additional centrifugation, and removal of the supernatant, T cells (1×10⁵) were added to the tubes and left in at 37° C. for 24 hours. Cells were then maintained in RPMI-1640 with FBS, antibiotics and 100 IU/mL IL-2 until the time of the experiments, 7-21 days after transduction.

Surface expression of CD16 was analyzed by flow cytometry using R-Phycoerythrin conjugated anti-human CD16 (clone B73.1, BD Biosciences Pharmingen, San Diego, Calif.). For western blotting, 2×10⁷ T cells were lysed in Cellytic M lysis Buffer (Sigma, St Louis, MO) containing 1% protease inhibitor cocktail (Sigma) and 1% phosphatase inhibitor cocktail 2 (Sigma). After centrifugation, lysate supernatants were boiled with an equal volume of LDS buffer (Invitrogen, Carlsbad, CA) with or without reducing buffer (Invitrogen) and then separated by NuPAGE Novex 12% Bis-Tris Gel (Invitrogen). The proteins were transferred to a polyvinylidene fluoride (PVDF) membrane, which was incubated with a mouse anti-human CD3ζ (clone 8D3; BD eBioscience Pharmingen) and then with a goat anti-mouse IgG horseradish peroxidase-conjugated secondary antibody (Cell Signaling Technology, Danvers, Mass.). Antibody binding was revealed by using the Amersham ECL Prime detection reagent (GE Healthcare).

mRNA Electroporation

The pVAX1 vector (Invitrogen, Carlsbad, Calif.) was used as a template for in vitro mRNA transcription. The CD16V-BB-ζ cDNA was subcloned into EcoRI and Xbal sites of pVAX1. The corresponding mRNA was transcribed in vitro with T7 mScript mRNA production system (CellScript, Madison, Wis.). Shimasaki et al., Cytotherapy. 2012;14(7):830-40.

For electroporation, the Amaxa Nucleofector (Lonza, Walkersville, Md.) was used; 1×10⁷ of purified T cells activated with 200 IU/mL IL-2 overnight were mixed with 200 μg/mL mRNA in Cell Line Nucleofector Kit V (Lonza), transferred into the processing chamber, and transfected using the program X-001. Immediately after electroporation, cells were transferred from the processing chamber into a 24-well plate and then cultured in RPMI-1640 with FBS, antibiotics and 100 IU/mL IL-2 (Roche, Mannheim, Germany). See also Shimasaki et al., Cytotherapy, 2012, 1-11.

### Antibody Binding, Cell Conjugation and Cell Proliferation Assays

To measure the chimeric receptors' antibody-binding capacity, T lymphocytes (5×10⁵) transduced with chimeric receptors or a vector containing GFP only were incubated with Rituximab (Rituxan, Roche; 0.1-1 μg/mL), Trastuzumab (Herceptin; Roche; 0.1-1 μg/mL) and/or purified human IgG (R&D Systems, Minneapolis, Minn. ; 0.1-1 μg/mL) for 30 minutes at 4° C. After washing twice with phosphate buffered saline (PBS), cells were incubated with goat anti-human IgG-PE (Southern Biotechnology Associates, Birmingham, Ala.) for 10 minutes at room temperature and cell staining was measured using an Accuri C6 flow cytometer (BD Biosciences).

To determine whether antibody binding to the receptor promoted cell aggregation, CD20-positive Daudi cells were labeled with CellTrace calcein red-orange AM (Invitrogen) and then incubated with Rituximab (0.1 μg/mL) for 30 minutes at 4° C. After washing twice in PBS, cells with Jurkat cells transduced with the chimeric receptor or mock-transduced at 1:1 E:T ratio in 96 round bottom plates (Costar, Corning, N.Y.) for 60 min at 37° C. The proportion of cells forming heterologous cell aggregates (calcein AM-GFP double positive) was determined by flow cytometry.

To measure cell proliferation, 1×10⁶ of T cells transduced with the chimeric receptor or mock-transduced were placed in the wells of a 24-well plate (Costar, Corning, N.Y.) in RPMI-1640 with FBS, antibiotics and 50 IU/mL IL-2. Daudi cells were treated with Streck cell preservative (Streck Laboratories, Omaha, Nebr.) to stop proliferation and labeled with Rituximab (0.1 μg/mL) for 30 min at 4° C. They were added to the wells, at 1:1 ratio with T cells, on days 0, 7, 14 and 21. The n number of viable T cells after culture was measured by flow cytometry.

### CD107 Degranulation and Cytotoxicity Assays

To determine whether CD16 cross-linking caused exocytosis of lytic granules, chimeric receptor- and mock transduced T cells (1×10⁵) were placed into each well of a Rituximab-coated 96-well flat bottom plate and cultured for 4 hours at 37° C. In other experiments, T cells were co-cultured with Daudi cells pre-incubated with Rituximab. An anti-human CD107a antibody conjugated to phycoerythrin (BD Biosciences) was added at the beginning of the cultures and one hour later GolgiStop (0.15 μl; BD Biosciences) was added. CD107a positive T cells were analyzed by flow cytometry.

To test cytotoxicity, target cells were suspended in RPMI-1640 with 10% FBS, labeled with calcein AM (Invitrogen) and plated into 96-well round bottom plates (Costar). T cells were added at various E: T ratio as indicated in Results, and co-cultured with target cells for 4 hours, with or without the antibodies Rituximab (Rituxan, Roche), Trastuzumab (Herceptin, Roche), or hu14.18K322A (obtained from Dr. James Allay, St Jude Children's GMP, Memphis, Tenn.; at 1 μg/mL). At the end of the cultures, cells were collected, resuspended in an identical volume of PBS, propidium iodide was added. The number of viable target cells (calcein AM-positive, propidium-iodide negative) was counted using the Accuri C6 flow cytometer.³⁴ For adherent cell lines, cytotoxicity was tested using luciferase-labeled target cells. To measure cytotoxicity against the adherent cell lines NB1691, CHLA-255, SK-BR-3, MCF-7, U-2 OS and MKN7, their luciferase-labeled derivatives were used. After plating for at least 4 hours, T cells were added as described above. After 4 hours of co-culture, the Promega Bright-Glo luciferase reagent (Promega, Madison, Wis.) was added to each well; 5 minutes later, luminescence was measured using a plate reader Biotek FLx800 (BioTek, Tucson, Ariz.) and analyzed with the GenS 2.0 Data Analysis Software.

### Xenograft Experiments

Daudi cells expressing luciferase were injected intraperitoneally (i.p.; 0.3×10⁶ cells per mouse) in NOD.Cg-Prkdc^{scid} IL2rg^{tm1wjl}/SzJ (NOD/scid IL2RGnull) mice (Jackson Laboratory, Bar Harbor). Some mice received Rituximab (100 μg) i.p. 4 days after Daudi inoculation, with or without i.p. injection of human primary T cells on days 5 and 6. T cells had been activated with anti-CD3/CD28 beads for 3 days, transduced with the CD16V-BB-ζ receptor, resuspended in RPMI-1640 plus 10% FBS and then injected at 1×10⁷ cells per mouse. Rituximab injection was repeated weekly for 4 weeks, with no further T lymphocyte injection. All mice received i.p. injections of 1000-2000 IU of IL-2 twice a week for 4 weeks. A group of mice received tissue culture medium instead of Rituximab or T cells.

Tumor engraftment and growth was measured using a Xenogen IVIS-200 system (Caliper Life Sciences, Hopkinton, Mass.). Imaging commenced 5 minutes after i.p. injection of an aqueous solution of D-luciferin potassium salt (3 mg/mouse) and photons emitted from luciferase-expressing cells were quantified using the Living Image 3.0 software.

### Results

Expression of the CD16V-BB-ζ receptor

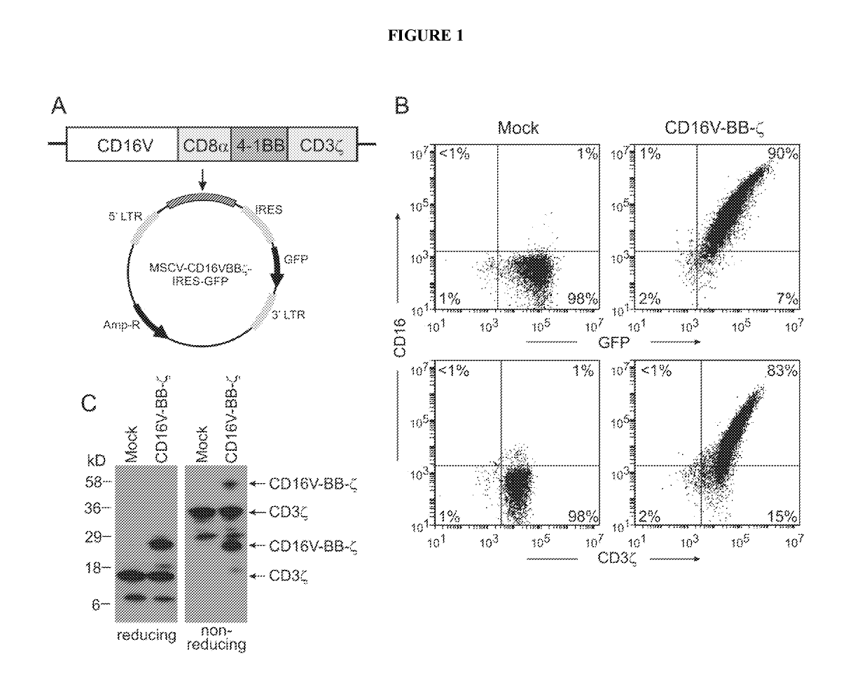

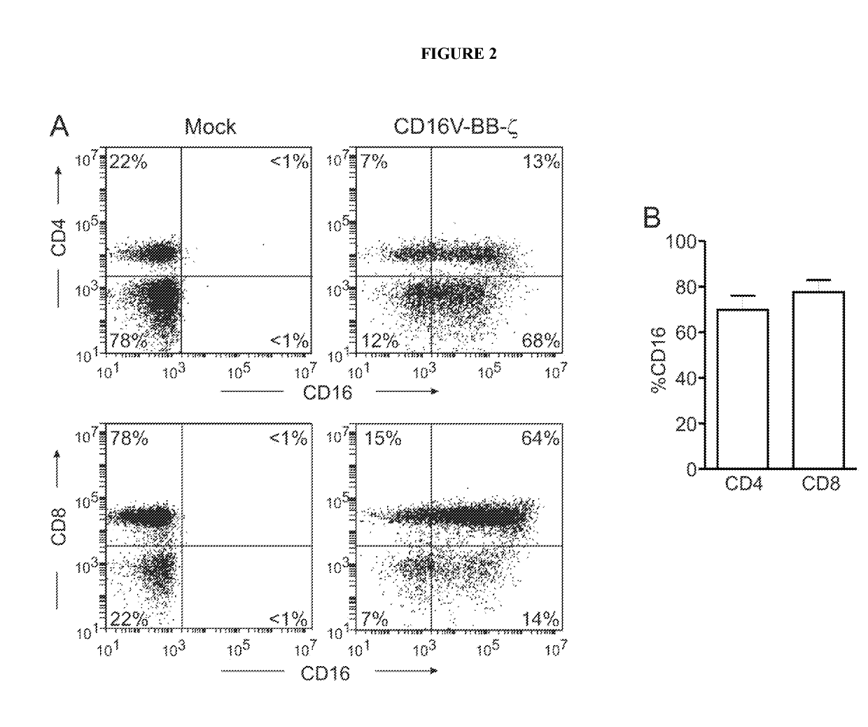

The V158 polymorphism of FCGR3A (CD16), expressed in about one-fourth of individuals, encodes a high-affinity immunoglobulin Fc receptor and is associated with favorable responses to antibody therapy . A V158 variant of the FCGR3A gene was combined with the hinge and transmembrane domain of CD8α, the T-cell stimulatory molecule CD3ζ, and the co-stimulatory molecule 4-1BB (FIG. 1A). An MCSV retroviral vector containing the CD16V-BB-ζ construct and GFP was used to transduce peripheral blood T lymphocytes from 12 donors: median GFP expression in CD3+cells was 89.9% (range, 75.3%-97.1%); in the same cells, median chimeric receptor surface expression as assessed by anti-CD16 staining was 83.0% (67.5%-91.8%) (FIG. 1B). T lymphocytes from the same donors transduced with a vector containing only GFP had a median GFP expression of 90.3% (67.8%-98.7%) but only 1.0% (0.1%-2.7%) expressed CD16 (FIG. 1B). Expression of the receptor did not differ significantly between CD4+ and CD8+ T cells: 69.8%±10.8% CD4+ cells were CD16+ after transduction with CD16V-BB-ζ, as compared to 77.6%±9.2% CD8+ cells (FIG. 2).

To ensure that the other components of the chimeric receptor were expressed, levels of expression of CD3 were measured by flow cytometry. As shown in FIG. 1B, CD16V-BB-ζ-transduced T lymphocytes expressed CD3 at levels much higher than those expressed by mock-transduced cells: mean (±SD) of the mean fluorescence intensity was 45,985±16,365 in the former versus 12,547±4,296 in the latter (P=0.027 by t test; n=3; FIG. 1B). The presence of the chimeric protein was also determined by western blotting probed with the anti-CD3 antibody. As shown in FIG. 1C, CD16V-BB-ζ-transduced T lymphocytes expressed a chimeric protein of approximately 25 kDa under reducing conditions, in addition to the endogenous CD3ζ of 16 kDa. Under non-reducing conditions, the CD16V-BB-ζ protein was shown to be expressed as either a monomer or a dimer of 50 kDa.

### Antibody-binding Capacity of V158 Versus F158 CD16 Receptors

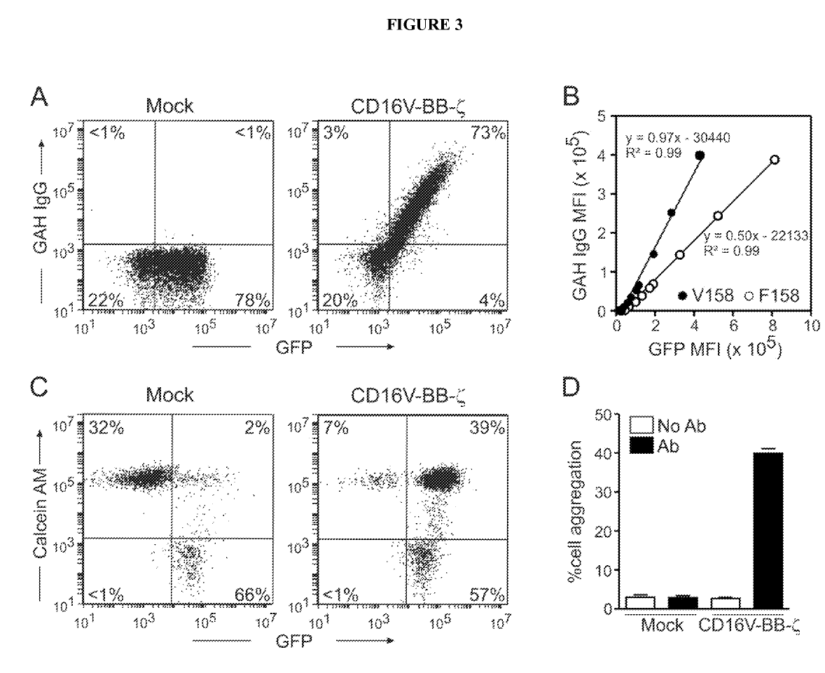

To test the capacity of the CD16V-BB-ζ chimeric receptor to bind immunoglobulin (Ig), peripheral blood T lymphocytes from 3 donors were transduced. As shown in FIG. 3A, CD16V-BB-ζ-expressing T lymphocytes were coated with the antibody after incubation with Rituximab. Similar results were obtained with Trastuzumab and human IgG.

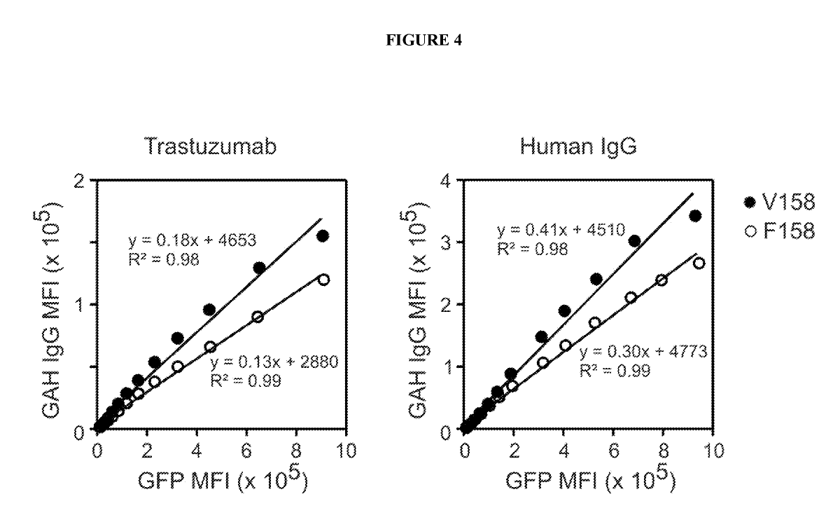

The Ig-binding capacity of the CD16V-BB-ζ receptor, which contained the high-affinity V158 polymorphism of FCGR3A (CD16), was then compared to that of an identical receptor containing the F158 variant instead (“CD16F-BB-ζ”). After transducing Jurkat cells with either receptor, they were incubated with Rituximab and an anti-human Ig PE antibody (binding Rituximab) and the PE fluorescence intensity was related to that of GFP. As shown in FIG. 3B, at any given level of GFP, cells transduced with the CD16V-BB-ζ receptor had a higher PE fluorescence intensity than that of cells transduced with the CD16F-BB-ζ receptor, indicating that the former had a significantly higher antibody-binding affinity. Trastuzumab and human IgG were also bound by CD16V-BB-ζ receptors with a higher affinity (FIG. 4).

To determine whether antibody binding to the CD16V-BB-ζ receptor could promote aggregation of effector and target cells, Jurkat cells expressing CD16V-BB-ζ (and GFP) were mixed at a 1:1 ratio with the CD20⁺ Daudi cell line (labeled with Calcein AM red-orange) for 60 minutes, and the formation of GFP-Calcein doublets was measured with or without addition of Rituximab. In 3 experiments, 39.0% ±1.9% of events in the coculture were doublets if Jurkat cells expressed CD16V-BB-ζ receptors and Rituximab was present (FIG. 3C and D). By contrast, there were <5% doublets with human IgG instead of Rituximab, or with mock-transduced Jurkat cells regardless of whether Rituximab was present.

### Binding of Ig to CD16V-BB-ζ Induces T Cell Activation, Degranulation and Cell Proliferation

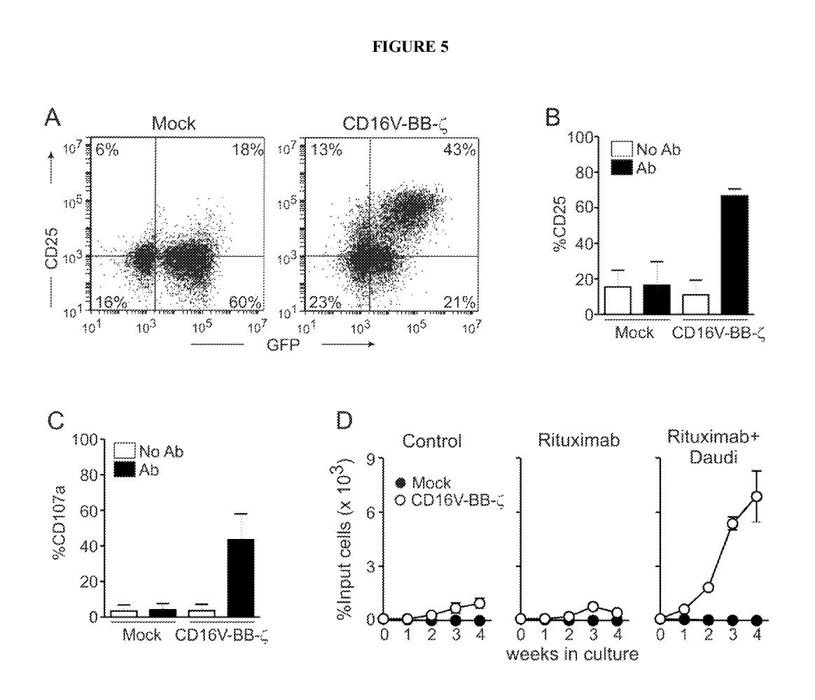

It was assessed whether CD16V-BB-ζ receptor cross-linking by an immobilized antibody could induce activation signals in T lymphocytes. Indeed, T lymphocytes transduced with CD16V-BB-ζ markedly increased IL-2 receptor expression (CD25) when cultured on plates coated with Rituximab whereas no changes were detected in the absence of antibody, or in mock-transduced cells regardless of whether the antibody was present (FIG. 5A and B).

In addition to expression of IL-2 receptors, CD16V-BB-ζ receptor cross-linking triggered exocytosis of lytic granules in T lymphocytes, as detected by CD107a staining. Thus, in 6 experiments in which T lymphocytes from 4 donors were either seeded onto microtiter plates coated with Rituximab (n=3) or cocultured with Daudi cells in the presence of Rituximab (n=3), T lymphocytes expressing CD16V-BB-ζ became CD107a positive (FIG. 5C).

Finally, it was determined whether receptor cross-linking could induce cell proliferation. As shown in FIG. 5D, T lymphocytes expressing CD16V-BB-ζ expanded in the presence of Rituximab and Daudi cells (at a 1 : 1 ratio with T lymphocytes): in 3 experiments, mean T cell recovery after 7 days of culture was 632% (±97%) of input cells; after 4 weeks of culture, it was 6877% (±1399%). Of note, unbound Rituximab, even at a very high concentration (1-10 μg/mL), had no significant effect on cell proliferation in the absence of target cells, and no cell growth occurred without Rituximab, or in mock-transduced T cells regardless of the presence of the antibody and/or target cells (FIG. 5D). Thus, CD16V-BB-ζ receptor cross-linking induces signals that result in sustained proliferation.

T lymphocytes Expressing CD16V-BB-ζ Mediate ADCC in vitro and in vivo

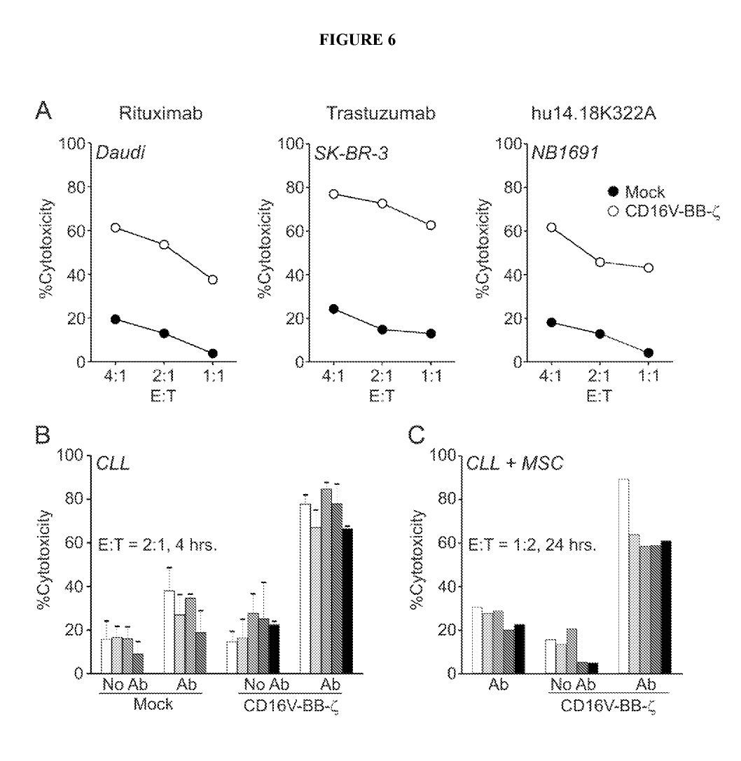

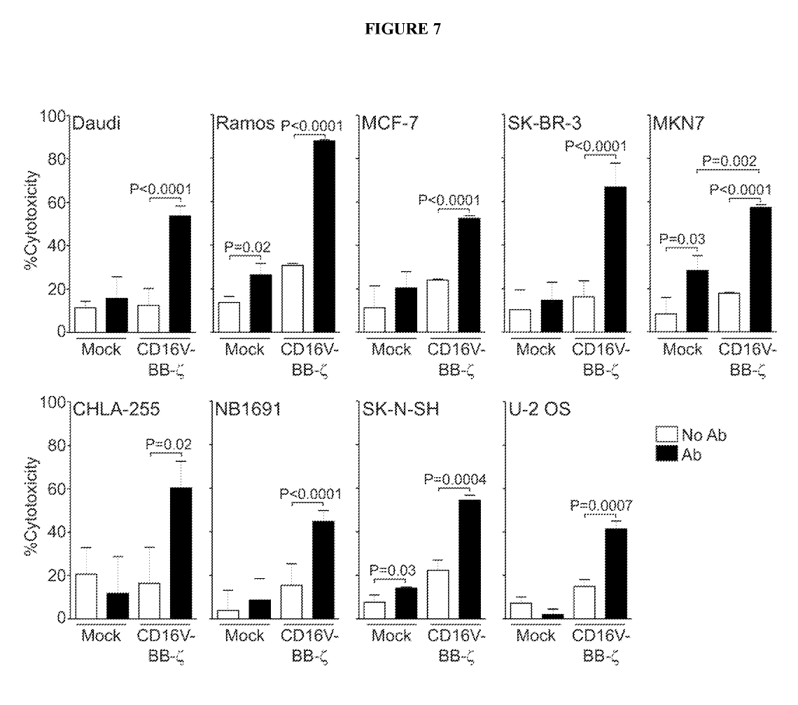

The observation that CD16V-BB-ζ cross-linking provoked exocytosis of lytic granules implied that CD16V-BB-ζ T lymphocytes should be capable of killing target cells in the presence of specific antibodies. Indeed, in 4-hour in vitro cytotoxicity assays, CD16V-BB-ζ T lymphocytes were highly cytotoxic against the B-cell lymphoma cell lines Daudi and Ramos in the presence of Rituximab: more than 50% target cells were typically lysed after 4 hours of co-culture at a 2 : 1 E : T ratio (FIG. 6 and FIG. 7). By contrast, target cell killing was low in the absence of the antibody or with mock-transduced T cells (FIG. 6 and FIG. 7). Notably, the effector cells used in these experiments were highly enriched with CD3+T lymphocytes (>98%) and contained no detectable CD3-CD56+ NK cells. Rituximab-mediated cytotoxicity of CD16V-BB-ζ T lymphocytes was also clear with CD20+primary CLL cells (n=5); as shown in FIG. 6B, cytotoxicity typically exceeded 70% after 4 hours of coculture at a 2:1 E:T ratio. Bone marrow mesenchymal stromal cells have been shown to exert immunosuppressive effects. To test whether this would affect the cytotoxic capacity of CD16V-BB-ζ T lymphocytes, they were co-cultured with CLL cells in the presence of bone marrow-derived mesenchymal stromal cells for 24 hours at a 1:2 E:T. As shown in FIG. 6C, mesenchymal cells did not diminish the killing capacity of the ADCC-mediating lymphocytes.

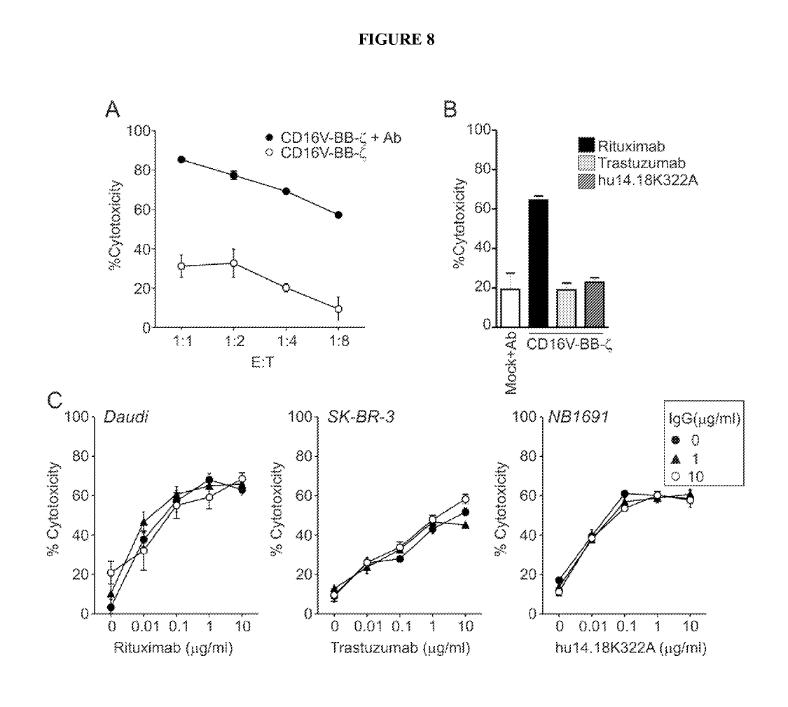

Next, it was investigated as to whether different immunotherapeutic antibodies could trigger similar cytotoxicities against tumor cells expressing the corresponding antigen. Thus, the cytotoxicity of CD16V-BB-ζ T lymphocytes was tested against solid tumor cells expressing HER2 (the breast cancer cell lines MCF-7 and SK-BR-3 and the gastric cancer cell line MKN7) or GD2 (the neuroblastoma cell lines CHLA-255, NB1691 and SK-N-SH, and the osteosarcoma cell line U2-OS). The antibodies Trastuzumab were used to target HER2 and hu14.18K322A were used to target GD2. CD16V-BB-ζ T lymphocytes were highly cytotoxic against these cells in the presence of the corresponding antibody (FIG. 6 and FIG. 7). In experiments with NB1691, it was also tested whether cytotoxicity could be achieved at even lower E:T ratios by prolonging the culture to 24 hours. As shown in Fig.8, cytotoxicity exceeded 50% at 1:8 ratio in the presence of hu14.18K322A. To further test the specificity of the CD16V-BB-ζ-mediated cell killing, the CD20+ Daudi cells were cultured with CD16V-BB-ζ T lymphocytes and antibodies of different specificity: only Rituximab mediated cytotoxicity, while there was no increase in cytotoxicity in the presence of Trastuzumab or hu14.18K322A (FIG. 8). Finally, it was determined whether CD16V-BB-ζ-mediated cell killing in the presence of immunotherapeutic antibodies could be inhibited by unbound monomeric IgG. As shown in FIG. 8, T cell cytotoxicity was not affected even if IgG was present at up to 1000 times higher concentration than the cell-bound immunotherapeutic antibody.

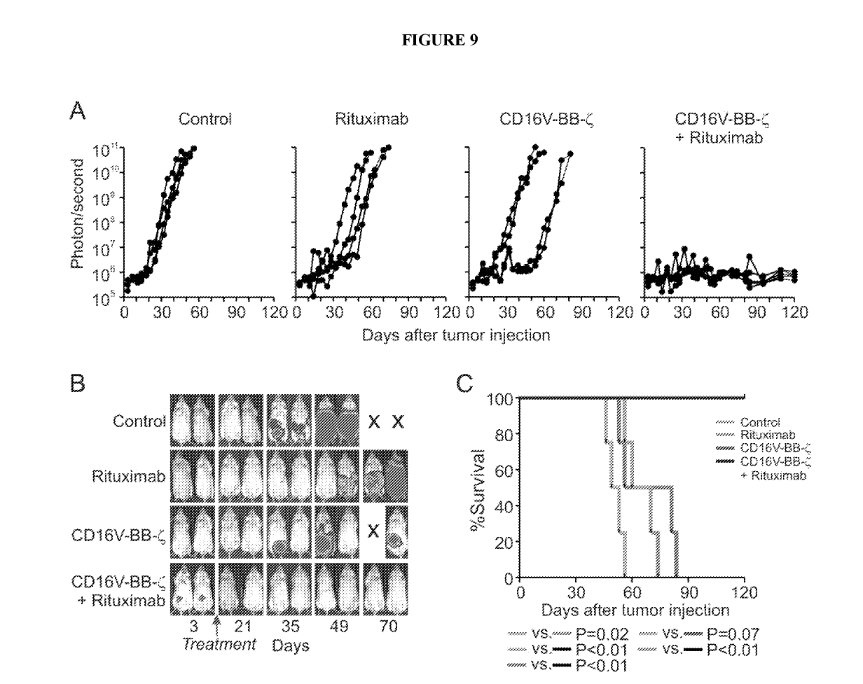

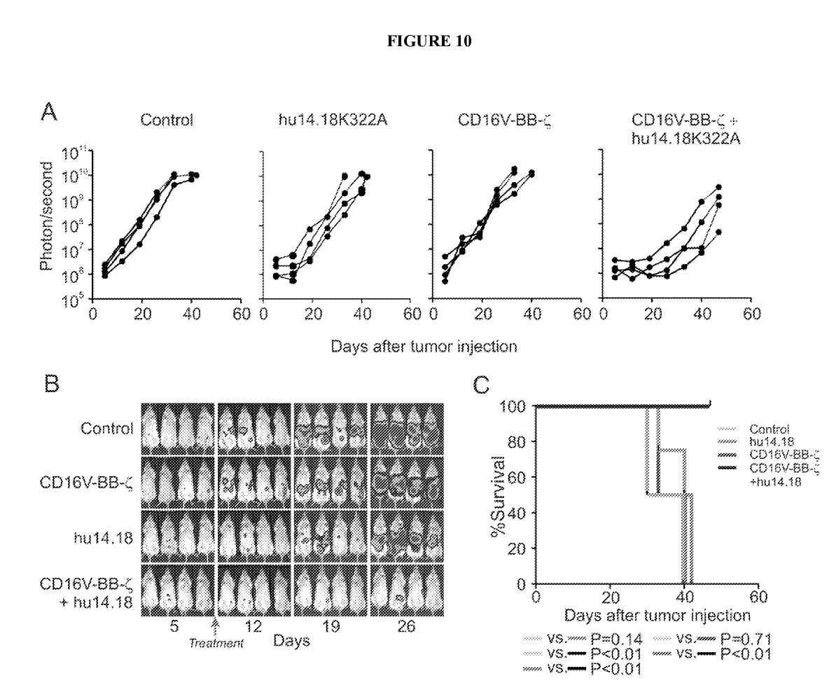

To gauge the anti-tumor capacity of CD16V-BB-ζ T lymphocytes in vivo, experiments were performed with NOD/scid IL2RGnull mice engrafted with luciferase-labeled Daudi cells. Tumor growth was measured by live imaging in mice receiving CD16V-BB-ζ T lymphocytes plus Rituximab, and their outcome was compared to mice receiving either Rituximab or T lymphocytes alone, or no treatment. As shown in FIG. 9, tumor cells expanded in all mice except those that received Rituximab followed by CD16V-BB-ζ T lymphocytes. All 5 mice treated with this combination were still in remission >120 days after tumor injection, in contrast to 0 of 12 mice that were untreated or received antibody or cells alone. A strong anti-tumor activity was also observed in mice engrafted with the neuroblastoma cell line NB1691 and treated with hu14.18K322A and CD16V-BB-ζ T lymphocytes (FIG. 10).

Comparison of CD16V-BB-ζ with other Receptors

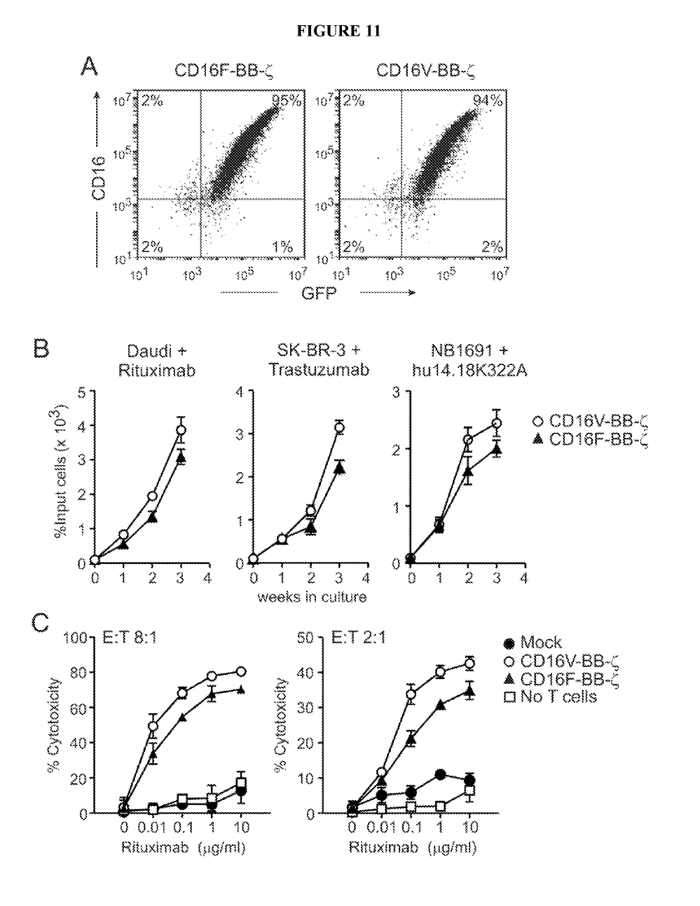

It was first compared the function of T cells bearing either CD16V-BB-ζ or CD16F-BB-ζ receptors. CD16F-BB-ζ receptors induced T cell proliferation and ADCC which was higher than that measured in mock-transduced T cells. Nevertheless, in line with their higher affinity for Ig, CD16V-BB-ζ receptors induced significantly higher T cell proliferation and ADCC than that triggered by the lower affinity CD16F-BB-ζ receptors (FIG. 11).

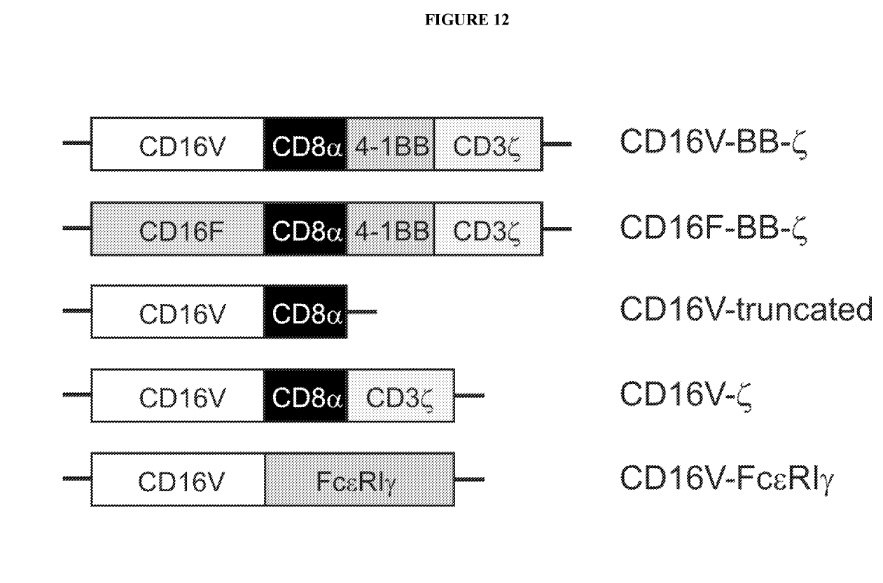

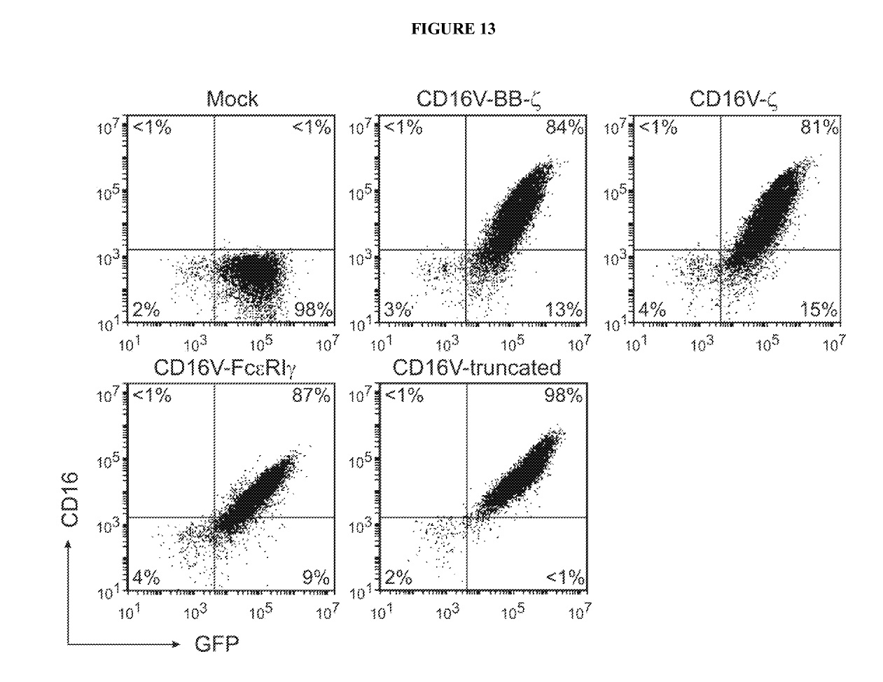

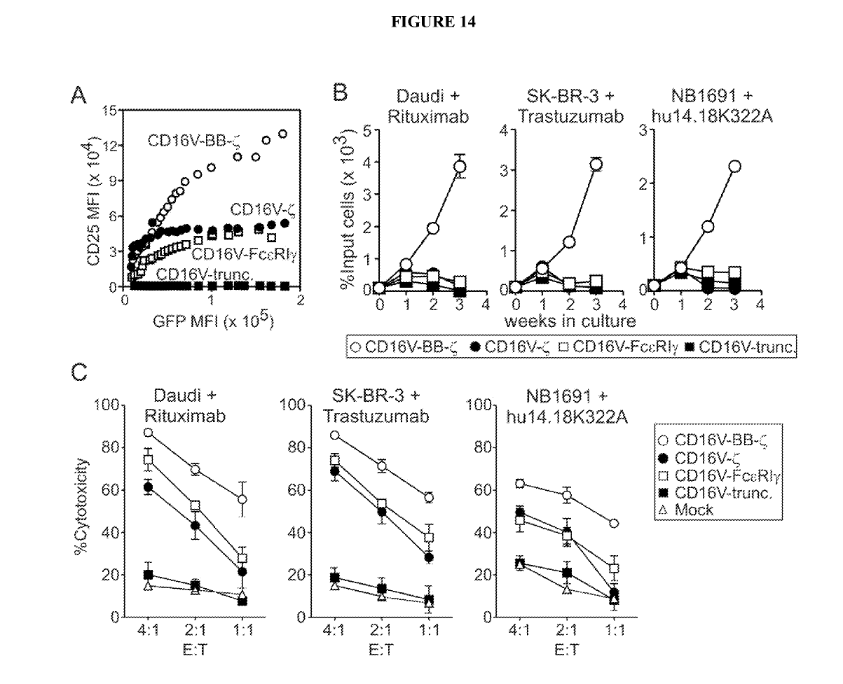

Next, the function of T cells bearing CD16V-BB-ζ was compared to that of T cells expressing other receptors with different signaling properties. These included a receptor with no signaling capacity (“CD16V-truncated”), one with CD3ζ but no 4-1BB (“CD16V-ζ”), and a previously described receptor that combined CD16V with the transmembrane and cytoplasmic domains of FcϵRIγ (“CD16V-FcϵRIγ”) (FIG. 12). After retroviral transduction in activated T cells, all receptors were highly expressed (FIG. 13). As shown in FIG. 14, CD16V-BB-ζ induced significantly higher activation, proliferation and specific cytotoxicity than all other constructs.

Expression of CD16V-BB-ζ Receptors by mRNA Electroporation

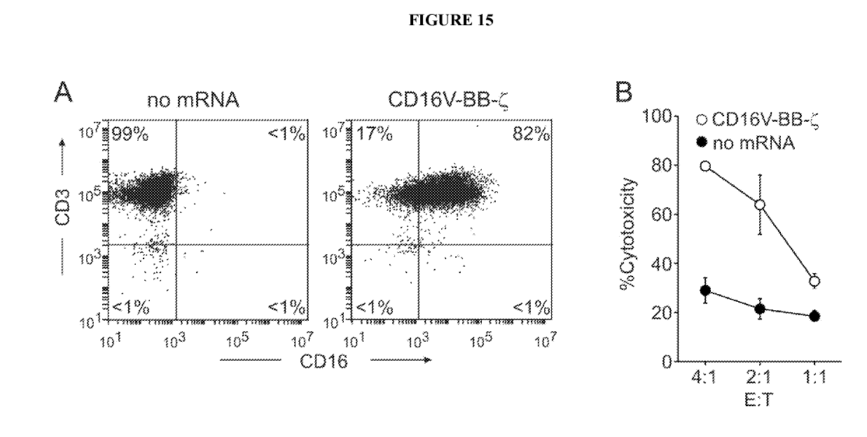

In all the above experiments, CD16V-BB-ζ expression was enforced by retroviral transduction. It was tested whether an alternative method, electroporation of mRNA, could also confer ADCC capacity to T lymphocytes. Activated T lymphocytes from 2 donors were electroporated and high expression efficiencies were obtained: 55% and 82% of T lymphocytes became CD16+ 24 hours after electroporation (FIG. 15A). In the second donor, receptor expression was also tested on day 3, when it was 43%, a result similar to those of previous experiments with another receptor where expression persisted for 72 to 96 hours . ADCC was activated in T lymphocytes electroporated with CD16V-BB-ζ mRNA: in the presence of Rituximab, 80% Ramos cells were killed after 4 hours at a 2 : 1 E : T ratio, while cells electroporated without mRNAs were ineffective (FIG. 15B).

See also Kudo et al., Cancer Res. 2014 Jan 1;74(1):93-103, the entire content of which is incorporated by reference herein.

### Discussion

Described herein is the development of chimeric receptors which endow T lymphocytes with the capacity to exert ADCC. When the CD16V-BB-ζ receptor is engaged by an antibody bound to tumor cells, it triggers T-cell activation, sustained proliferation and specific cytotoxicity against cancer cells targeted by antibody. CD16V-BB-ζ T lymphocytes were highly cytotoxic against a wide range of tumor cell types, including B-cell lymphoma, breast and gastric cancer, neuroblastoma and osteosarcoma, as well as primary CLL cells. Cytotoxicity was entirely dependent on the presence of a specific antibody bound to target cells; unbound antibodies did not provoke non-specific cytotoxicity nor affected cytotoxicity with cell-bound antibodies. CD16V-BB-ζ T cells also killed CLL cells when these were cultured on mesenchymal cell layers, regardless of the known immunosuppressive effects of this microenvironment. Moreover, CD16V-BB-ζ T lymphocytes infused after Rituximab eradicated B-cell lymphoma cells engrafted in immunodeficient mice, and had considerable anti-tumor activity in mice engrafted with neuroblastoma cells in the presence of an anti-GD2 antibody. In sum, T cells expressing CD16V-BB-ζ effected strong ADCC in vitro and in vivo.

The affinity of CD16 for the Fc portion of Ig is a critical determinant of ADCC and, thus, influences clinical responses to antibody immunotherapy. Hence, considerable efforts are being made to further enhance the affinity of Fc fragments for FcyR, for example by glycoengineering . To construct the chimeric receptor of the disclosure, the FCGR3A (CD16) gene with the V158 polymorphism (SEQ ID NO: 65) was selected as an example. This variant encodes a receptor with higher binding affinity for Ig and has been shown to mediate superior ADCC . Indeed, in side-to-side comparisons with an identical chimeric receptor containing the more common F158 variant, the CD16V-BB-ζ had a significantly higher capacity to bind human Ig Fc, and induced more vigorous proliferation and cytotoxicity, evoking results of recent studies addressing the role of affinity in chimeric antigen receptor function . Current “second generation” chimeric receptors combine a stimulatory molecule with a co-stimulatory one to augment signaling and prevent activation-induced apoptosis. Therefore, CD16 V158 was combined with a stimulatory molecular tandem constituted by CD3ζ and 4-1BB (CD137). Indeed, the CD16V-BB-ζ receptor induced a markedly superior T cell activation, proliferation and cytotoxicity than did receptors acting through CD3 alone, or of FcϵRIγ.

The clinical potential of genetically modified T cells expressing receptors that recognize antigens of the surface of tumor cells and can transduce stimulatory signals is being increasingly demonstrated by results of clinical trials. Most notably, significant tumor reductions and/or complete remissions have been reported in patients with B-cell malignancies who received autologous T lymphocytes expressing chimeric antigen receptors against CD19 or CD20 by viral transduction . Expanding this strategy to other tumors involves considerable effort, including the development of another chimeric antigen receptor construct, and the optimization of large-scale transduction conditions in compliance with regulatory requirements. In this regard, the CD16V-BB-ζ receptor described herein should facilitate the implementation of T-cell therapy by allowing one single receptor to be used for multiple cancer cell types. It should also allow the targeting of multiple antigens simultaneously, a strategy that may ultimately be advantageous given immunoescape mechanisms exploited by tumors, as illustrated by the recent report of a leukemia relapse driven by a subclone lacking the marker targeted by a chimeric receptor with single specificity. Antibody-directed cytotoxicity could be stopped whenever required by simple withdrawal of antibody administration. Because the T cells expressing CD16V-BB-ζ are only activated by antibody bound to target cells, soluble immunoglobulin should not exert any stimulation on the infused T cells. As demonstrated herein, mRNA electroporation can express the receptor very effectively.

Antibody therapy has become standard-of-care for many cancer subtypes; its clinical efficacy is mostly determined by its capacity to trigger ADCC through the engagement of Fc receptors. The main effectors of ADCC are NK cells but their function can be impaired in patients with cancer. For example, it was reported that Trastuzumab-mediated ADCC of gastric cancer cells overexpressing HER2 was significantly lower with peripheral blood mononuclear cells from gastric cancer patients and advanced disease as compared to that obtained with samples from patients with early disease or healthy donors. Moreover, responses are likely to be influenced by other factors, including the genotype of NK-cell inhibitory receptors and their ligands . The results presented herein suggest that the infusion of autologous T cells genetically engineered with the CD16V-BB-ζ receptor should significantly boost ADCC. Because the combined CD3ζ/4-1BB signaling also causes T-cell proliferation, there should be an accumulation of activated T cells at the tumor site which may further potentiate their activity. CD16V-BB-ζ receptors can be expressed by mRNA electroporation not only in activated T lymphocytes but also in resting peripheral blood mononuclear cells, a procedure that would take only a few hours from blood collection to infusion of CD16V-BB-ζ-expressing cells and is therefore well suited for clinical application.

### Example 2

### Construction of Various Chimeric Receptors

Nucleic acid sequences encoding chimeric receptors SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, and SEQ ID NO: 14 were cloned into the HindIII and Xbal sites of vector pVAX1. The DNA vectors were linearized by digestion with restriction endonuclease XbaI and transcribed into RNA with T7 RNA polymerase. The RNA was subsequently enzymatically capped at its 5′-end with ScriptCap Capping Enzyme and ScriptCap 2′-O-Methyltransferase from Cellscript to give a Cap 1 structure and then poly-adenylated at its 3′-end with poly-A polymerase. The resulting mRNA was electroporated into Jurkat cells using an Invitrogen Neon electroporation system and grown in RPMI-1640 media with 10% fetal bovine serum at 37° C. for 6 hr.

Electroporated cells in media were then incubated with the CD20-specific antibody Rituxan (10 μg/mL) at 37° C. for 30 min. Cells were harvested, washed twice with flow cytometry buffer (FC buffer; DPBS without Ca2+and Mg2+, 0.2% bovine serum albumin, 0.2% NaN3) and stained with a PE-labeled anti-CD16 antibody or anti-CD32 antibody (for SEQ ID NO: 6) to detect chimeric receptor expression or a PE-labeled goat-anti-human antibody to detect bound Rituxan. Stained cells were analyzed by flow cytometry. Chimeric receptor proteins from all constructs were detected with the PE-labeled anti-CD16 or anti-CD32 antibodies with mean fluorescence values that ranged from 36,000 to 537,000. Uconstruct 1 (SEQ ID NO: 1) showed the highest expression level.

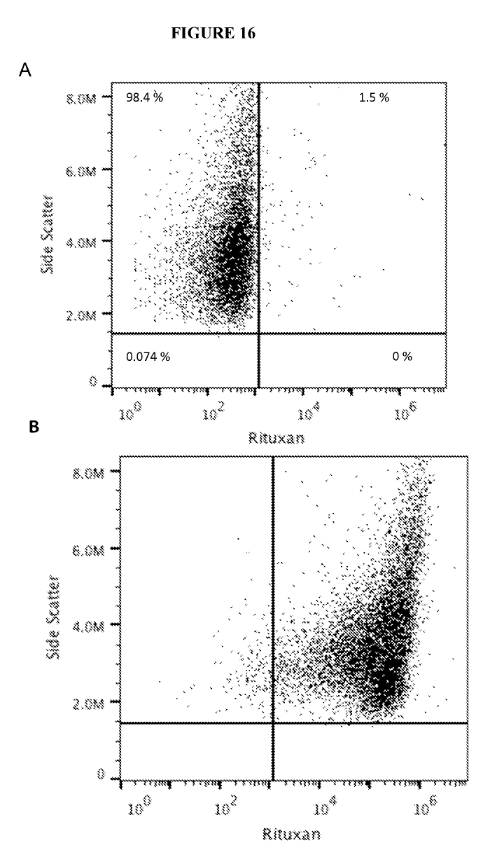

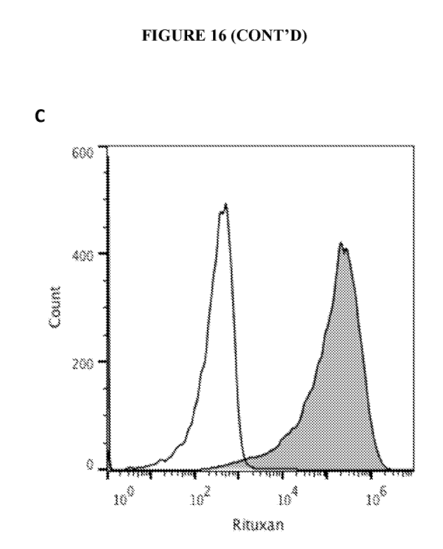

FIG. 16 (panels A to C) shows flow cytometry data for Rituxan binding to cells electroporated with SEQ ID NO: 1 mRNA and mock-electroporated cells. Greater than 95% of the cells electroporated with mRNA encoding SEQ ID NO: 1 were stained with the goat-anti-human antibody, as compared to less than 2% of mock-electroporated cells, indicating the chimeric receptor expressed on the surface of Jurkat cells was able to bind to Rituxan (FIGS. 16A and 16B). The median fluorescence value of SEQ ID NO: 1 mRNA-electroporated cells was approximately 700-fold higher than the median fluorescence value of mock-electroporated cells when stained with PE-labeled goat-anti-human antibody (FIG. 16C, Table 8). A similar analysis was carried out for SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9), SEQ ID NO: 10, SEQ ID NO: 11, and SEQ ID NO: 14. The median fluorescence values for cells electroporated with chimeric receptor mRNA for these constructs were approximately 14- to 680-fold higher than the median fluorescence value of mock-electroporated cells when stained with PE-labeled goat-anti-human antibody (Table 8), indicating that all of these chimeric receptor proteins that were expressed on the surface of Jurkat cells were able to bind to Rituxan.

These experiments indicated that chimeric receptors SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, and SEQ ID NO: 14 were all expressed in Jurkat cells and all bound the CD20-specific antibody Rituxan.

TABLE 8
Relative median fluorescence for chimeric receptor binding to Rituxan, and
CD25 and CD69 expression in activity experiments for chimeric receptor constructs.

| | | | Rituxan | CD25 | CD69 |
|---|---|---|---|---|---|
| | | | binding | expression | expression |
| mRNA | | | relative | relative | relative |
| electroporated into | | variation relative to | median | median | median |
| Jurkat cells | SEQ ID | SEQ ID NO: 1 | fluorescence | fluorescence | fluorescence |
| none | — | — | 1 | 1 | 1 |
| SEQ ID NO: 1 | 1 | — | 694.7 | 6.7 | 69.1 |
| SEQ ID NO: 2 | 2 | 4-1BB TM domain | 22.4 | 2.3 | 10.9 |
| SEQ ID NO: 3 | 3 | CD28 TM domain | 77.7 | 5.1 | 25.0 |
| SEQ ID NO: 4 | 4 | CD34 TM domain | 25.8 | 3.0 | 10.6 |
| SEQ ID NO: 5 | 5 | generic TM domain | 14.2 | 2.4 | 10.2 |
| SEQ ID NO: 6 | 6 | CD32 Fc receptor | 682.4 | 5.4 | 64.1 |
| SEQ ID NO: 7 | 7 | CD28 costimulatory | 232.7 | 4.7 | 35.2 |
| | | domain | | | |
| SEQ ID NO: 8 | 8 | OX40 costimulatory | 322.7 | 5.3 | 37.6 |
| | | domain | | | |
| SEQ ID NO: 9 | 9 | CD28 + 4-1BB | 102.4 | 5.3 | 25.7 |
| | | costimulatory | | | |
| | | domains | | | |
| SEQ ID NO: 10 | 10 | no hinge | 24.0 | 5.8 | 13.0 |
| SEQ ID NO: 11 | 11 | XTEN hinge | 55.4 | 7.6 | 20.6 |
| SEQ ID NO: 14 | 14 | CD4 TM domain | 32.1 | 4.8 | 13.0 |

### Example 3

### Cells Expressing Chimeric Receptors Display T cell Activation Markers

Jurkat cells expressing the chimeric receptors disclosed in Example 2 above were evaluated for activity by monitoring for the presence of the cell-surface activity markers CD25 and CD69. For these experiments, Jurkat cells were electroporated without mRNA (mock) or with mRNA encoding the chimeric receptor constructs described in Example 2 above, using an Invitrogen Neon electroporation system and grown in RPMI-1640 media with 10% FBS at 37° C. for 8-9 hr. Cells were harvested, washed with RPMI-1640 media with 10% fetal bovine serum, 50 U/mL penicillin, and 50 μg/mL streptomycin. These cells were mixed at a one to one ratio with Daudi target cells, which have cell-surface-expressed CD20, that had been fixed with Streck's cell preservative, and the CD20-specific antibody Rituxan (10 μg/mL). This mixture was incubated at 37° C. for 18-20 hr in RPMI-1640 media with 10% fetal bovine serum, 50 U/mL penicillin, and 50 μg/mL streptomycin. Cells were harvested and stained with a PE-labeled anti-CD7 antibody to detect Jurkat cells and an APC-labeled anti-CD25 antibody or an APC-labeled anti-CD69 antibody to detect CD25 and CD69 expression, respectively, on Jurkat cells. Stained cells were evaluated by flow cytometry.

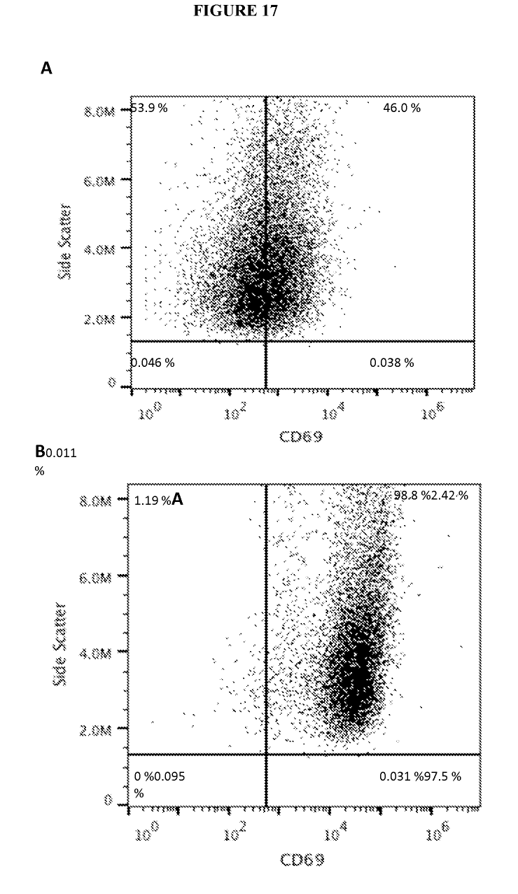

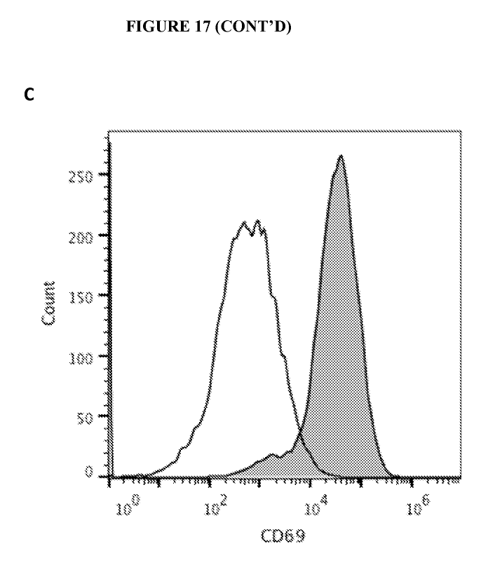

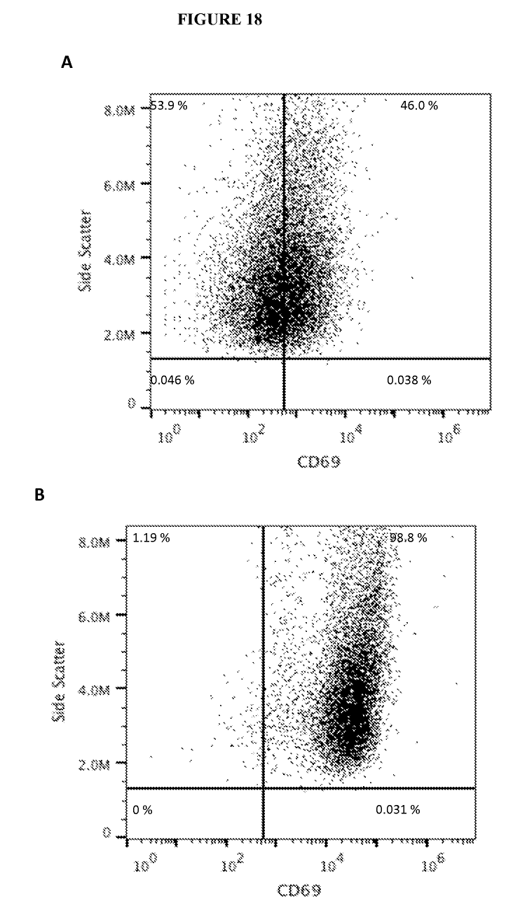

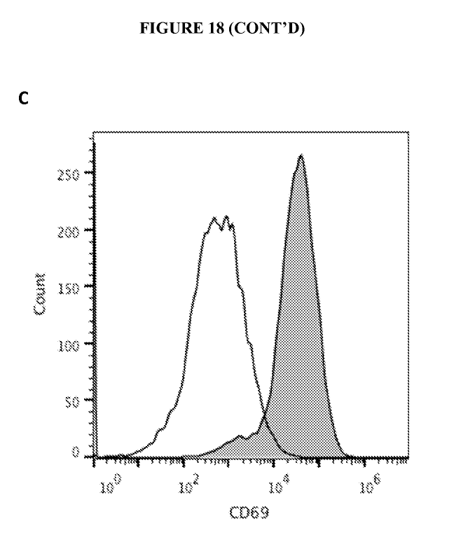

CD7 positive cells were evaluated for expression of both CD25 and CD69. Greater than 45% of the CD7-positive cells in the condition with mRNA encoding SEQ ID NO: 1 were stained with the APC-labeled anti-CD25 antibody, as compared to less than 3% of CD7-positive cells in the mock-electroporation condition, indicating increased expression of the CD25 activity marker on Jurkat cells expressing chimeric receptor versus cells that do not express the receptor under the conditions of these experiments (FIGS. 17A and 17B). The median fluorescence value in the SEQ ID NO: 1 mRNA condition was approximately 6.7-fold higher than the median fluorescence value of mock-electroporated cells when CD7-positive cells were evaluated for staining with APC-labeled anti-CD25 antibody (FIG. 17C, Table 8). Greater than 98% of the CD7-positive cells in the condition with mRNA encoding SEQ ID NO: 1 were stained with the APC-labeled anti-CD69 antibody, as compared to approximately 46% of CD7-positive cells in the mock-electroporation condition, indicating increased expression of the CD69 activity marker on Jurkat cells expressing chimeric receptor versus cells that do not express the receptor under the conditions of these experiments (FIGS. 18A and 18B). The median fluorescence value in the SEQ ID NO: 1 mRNA condition was approximately 69-fold higher than the median fluorescence value of mock-electroporated cells when CD7-positive cells were evaluated for staining with APC-labeled anti-CD69 antibody (FIG. 18C, Table 8).

A similar analysis was carried out for SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, and SEQ ID NO: 14. The median fluorescence values for conditions in which cells were expressing chimeric receptors for these constructs were approximately 2.3- to 7.6-fold higher than the median fluorescence value of mock-electroporated cells when CD7-positive cells were evaluated for staining with APC-labeled anti-CD25 antibody (Table 8), indicating increased expression of the CD25 activity marker on Jurkat cells expressing each of these chimeric receptor versus cells that do not express the receptor under the conditions of these experiments (Table 8). The median fluorescence values for conditions in which cells were expressing chimeric receptors for these constructs were approximately 10- to 64-fold higher than the median fluorescence value of mock-electroporated cells when CD7-positive cells were evaluated for staining with APC-labeled anti-CD69 antibody (Table 8), indicating increased expression of the CD69 activity marker on Jurkat cells expressing each of these chimeric receptor versus cells that do not express the receptor under the conditions of these experiments (Table 8).

These experiments indicate that Jurkat cells expressing these chimeric receptors show an increase in the activity markers CD25 and CD69 relative to Jurkat cells that do not express a chimeric receptor under conditions where these receptors interact with the CD20-specific antibody Rituxan and CD20-expressing Daudi target cells.

### Example 4

### Chimeric Receptors Are Expressed on Jurkat Cells

Jurkat cells electroporated with mRNA encoding chimeric receptors were analyzed for chimeric receptor expression by Western blot analysis with an anti-CDζ antibody. For these experiments, Jurkat cells were electroporated without mRNA (mock) or with mRNA encoding the constructs disclosed in Example 2 above, using an Invitrogen Neon electroporation system and grown in RPMI-1640 media with 10% FBS at 37° C. for 8 - 9 hr. Cells were harvested and lysed with RIPA buffer (50 mM Tris-HCl, 150 mM NaCl, 1 mM EDTA, 1% NP-40, 0.5% sodium deoxycholate, pH 7.4) in the presence of phosphatase and protease inhibitors. For each lysate, 25 μg of total protein was loaded onto one lane of a 4-12% Bis-Tris polyacrylamide gel. Proteins were transferred to a PVDF membrane and the membrane was blocked with 5% milk in TBST buffer (500 mM Tris-HCl, 1.5M NaCl, 1% Tween-20, pH 7.4) for 1 hr at room temperature. The membrane was probed with an anti-CDζ antibody overnight at 4° C., washed 3 times with TBST buffer, and probed with a horseradish-peroxidase-linked goat-anti-human secondary antibody. Protein bands were visualized using a horseradish peroxidase chemiluminescent substrate.

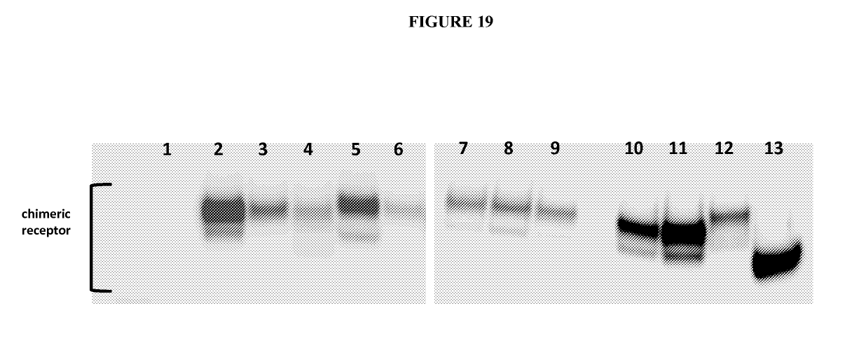

The results of the Western blot experiments are shown in FIG. 19. The anti-CDζ antibody detects the C-terminal region of the chimeric receptor proteins that contain the CDζ intracellular protein sequence. For all chimeric receptor constructs, bands corresponding to the full-length receptor protein were detected (lanes 2-13). The mobility of the chimeric receptor proteins varies in a manner that is consistent with the different molecular weights of the proteins.

These results demonstrate that these chimeric receptors were all expressed in Jurkat cells after electroporation with the corresponding mRNA.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one of skill in the art can easily ascertain the essential characteristics of the present disclosure, and without departing from the spirit and scope thereof, can make various changes and modifications of the disclosure to adapt it to various usages and conditions. Thus, other embodiments are also within the claims.

1. A chimeric receptor, comprising:
(a) an Fc binding domain;
(b) a transmembrane domain;
(c) at least one co-stimulatory signaling domain; and
(d) a cytoplasmic signaling domain comprising an immunoreceptor tyrosine-based activation motif (ITAM); wherein either (c) or (d) is located at the C-terminus of the chimeric receptor; and wherein
(i) if (a) is an extracellular ligand-binding domain of CD16A, (d) does not comprise an ITAM domain of an Fc receptor, and
(ii) the chimeric receptor is not a receptor comprising, from N-terminus to C-terminus, an extracellular ligand-binding domain of F158 CD16A or V158 CD16A, a hinge and transmembrane domain of CD8a, a co-stimulatory signaling domain of 4-1BB, and a cytoplasmic signaling domain of CD3ζ.

2. The chimeric receptor of claim 1, wherein (d) is located at the C-terminus of the chimeric receptor.

3. The chimeric receptor of claim 1, further comprising (e) a hinge domain, which is located at the C-terminus of (a) and the N-terminus of (b).

4. The chimeric receptor of claim 1, wherein the chimeric receptor further comprises a signal peptide at its N-terminus.

5. The chimeric receptor of claim 1, wherein the Fc binding domain of (a) is selected from the group consisting of:
(i) an extracellular ligand-binding domain of an Fc-receptor, which optionally is an Fc-gamma receptor, an Fc-alpha receptor, or an Fc-episilon receptor,
(ii) an antibody fragment that binds the Fc portion of an immunoglobulin,
(iii) a naturally-occurring protein that binds the Fc portion of an immunoglobulin or an Fc-binding fragment thereof, and
(iv) a synthetic polypeptide that binds the Fc portion of an immunoglobulin.

6. The chimeric receptor of claim 5, wherein the Fc binding domain is (i), which is an extracellular ligand-binding domain of CD16A, CD32A, or CD64A.

7. The chimeric receptor of claim 6, wherein the Fc binding domain is an extracellular ligand-binding domain of CD32A or CD64A.

8. The chimeric receptor of claim 5, wherein the Fc binding domain is (ii), which is a single chain variable fragment (ScFv), a domain antibody, or a nanobody.

9. The chimeric receptor of claim 5, wherein the Fc binding domain is (iii), which is Protein A or Protein G.

10. The chimeric receptor of claim 5, wherein the Fc binding domain is (iv), which is a Kunitz peptide, a SMIP, an avimer, an affibody, a DARPin, or an anticalin.

11. The chimeric receptor of claim 1, wherein the transmembrane domain of (b) is of a single-pass membrane protein.

12. The chimeric receptor of claim 11, wherein the transmembrane domain is of a membrane protein selected from the group consisting of CD8α, CD8β, 4-1BB, CD28, CD34, CD4, FcϵRIγ, CD16A, OX40, CD3ζ, CD3ϵ, CD3γ, CD3δ, TCRα, CD32, CD64, VEGFR2, FAS, and FGFR2B.

13. The chimeric receptor of claim 11, wherein the transmembrane domain is of a membrane protein selected from the group consisting of CD8β, 4-1BB, CD28, CD34, CD4, FcϵRIγ, CD16A, OX40, CD3ζ, CD3ϵ, CD3γ, CD3δ, TCRα, CD32, CD64, VEGFR2, FAS, and FGFR2B.

14. The chimeric receptor of claim 1, wherein the transmembrane domain of (b) is a non-naturally occurring hydrophobic protein segment.

15. The chimeric receptor of claim 1, wherein the at least one co-stimulatory signaling domain of (c) is of a co-stimulatory molecule selected from the group consisting of 4-1BB, CD28, CD28_{LL→GG} variant, OX40, ICOS, CD27, GITR, HVEM, TIM1, LFA1, and CD2.

16. The chimeric receptor of claim 1, wherein the at least one co-stimulatory signaling domain of (c) is of a co-stimulatory molecule selected from the group consisting of CD28, CD28_{LL→GG} variant, OX40, ICOS, CD27, GITR, HVEM, TIM1, LFA1, and CD2.

17. The chimeric receptor of claim 1, wherein the chimeric receptor comprises two co-stimulatory signaling domains.

18. The chimeric receptor of claim 17, wherein the two co-stimulatory domains are:
(i) CD28 and 4-1BB; or
(ii) CD28_{LL→GG} variant and 4-1BB.

19. The chimeric receptor of claim 1, wherein the cytoplasmic signaling domain of (d) is a cytoplasmic domain of CD3 or FcϵR1γ.

20. The chimeric receptor of claim 3, wherein the hinge domain of (e) is of CD8α or IgG.

21. The chimeric receptor of claim 3, wherein the hinge domain is a non-naturally occurring peptide.

22. The chimeric receptor of claim 21, wherein the hinge domain is an extended recombinant polypeptide (XTEN) or a (Gly₄Ser)ₙ polypeptide, in which n is an integer of 3-12, inclusive.

23. The chimeric receptor of claim 3, wherein the chimeric receptor comprises components (a)-(e) as shown in Table 3.

24. The chimeric receptor of claim 23, wherein the chimeric receptor comprises the amino acid sequence selected from SEQ ID NOs:2-11.

25. The chimeric receptor of claim 3, wherein the chimeric receptor comprises components (a)-(e) as shown in Table 4.

26. The chimeric receptor of claim 25, wherein the chimeric receptor comprises the amino acid sequence selected from SEQ ID NOs:12-17.

27. The chimeric receptor of claim 3, wherein the chimeric receptor comprises components (a)-(e) as shown in Table 5.

28. The chimeric receptor of claim 27, wherein the chimeric receptor comprises the amino acid sequence selected from SEQ ID NOs:18-30, and 32-56.

29. A nucleic acid comprising a nucleotide sequence encoding a chimeric receptor of claim 1.

30. The nucleic acid of claim 29, wherein the nucleic acid is an RNA molecule.

31. A vector comprising the nucleic acid of claim 29.

32. The vector of claim 31, wherein the vector is an expression vector.

33. The vector of claim 32, wherein the vector is a viral vector.

34. The vector of claim 33, wherein the viral vector is a lentiviral vector or a retroviral vector.

35. A host cell, comprising a nucleic acid of claim 29.

36. The host cell of claim 35, wherein the host cell is an immune cell.

37. The host cell of claim 36, wherein the immune cell is a natural killer cell, macrophage, neutrophil, eosinophil, or T cell.

38. The host cell of claim 35, wherein the host cell is a T cell in which the expression of the endogenous T cell receptor has been inhibited or eliminated.

39. A pharmaceutical composition, comprising (a) a host cell of claim 35 and (b) a pharmaceutically acceptable carrier.

40. The pharmaceutical composition of claim 39, wherein the composition further comprises an Fc-containing therapeutic agent.

41. The pharmaceutical composition of claim 40, wherein the Fc-containing therapeutic agent is an Fc fusion protein.

42. The pharmaceutical composition of claim 40, wherein the Fc-containing therapeutic agent is a therapeutic antibody or an Fc fusion protein.

43. The pharmaceutical composition of claim 42, wherein the Fc-containing therapeutic agent is a therapeutic antibody selected from the group consisting of Adalimumab, Ado-Trastuzumab emtansine, Alemtuzumab, Basiliximab, Bevacizumab, Belimumab, Brentuximab, Canakinumab, Cetuximab, Certolizumab, Daclizumab, Denosumab, Dinutuximab, Eculizumab, Efalizumab, Epratuzumab, Gemtuzumab, Golimumab, Ibritumomab, Infliximab, Ipilimumab, Labetuzumab, Muromonab, Natalizumab, Obinutuzumab, Ofatumumab, Omalizumab, Palivizumab, Panitumumab, Pertuzumab, Ramucirumab, Ranibizumab, Rituximab, Tocilizumab, Trastuzumab, Tositumomab. Ustekinumab, and Vedolizumab.

44. A kit, comprising:
a first pharmaceutical composition that comprises (i) a host cell of claim 35 and (ii) a pharmaceutically acceptable carrier; and
a second pharmaceutical composition that comprises an Fc-containing therapeutic agent and a pharmaceutically acceptable carrier.

45. The kit of claim 44, wherein the Fc-containing therapeutic agent is an Fc fusion protein.

46. The kit of claim 44, wherein the Fc-containing therapeutic agent is a therapeutic antibody.

47. The kit of claim 46, wherein the therapeutic antibody is selected from the group consisting of Adalimumab, Ado-Trastuzumab emtansine, Alemtuzumab, Basiliximab, Bevacizumab, Belimumab, Brentuximab, Canakinumab, Cetuximab, Certolizumab, Daclizumab, Denosumab, Dinutuximab, Eculizumab, Efalizumab, Epratuzumab, Gemtuzumab, Golimumab, Ibritumomab, Infliximab, Ipilimumab, Labetuzumab, Muromonab, Natalizumab, Obinutuzumab, Ofatumumab, Omalizumab, Palivizumab, Panitumumab, Pertuzumab, Ramucirumab, Ranibizumab, Rituximab, Tocilizumab, Trastuzumab, Tositumomab. Ustekinumab, and Vedolizumab.

48. A method for enhancing antibody-dependent cell-mediated cytotoxicity (ADCC) or for enhancing efficacy of an antibody-based immunotherapy in a subject, comprising administering to the subject an effective amount of host immune cells that express a chimeric receptor of claim 1.

49. The method of claim 48, wherein the host immune cells are natural killer cells, macrophages, neutrophils, eosinophils, T cells, or a combination thereof.

50. The method of claim 48, wherein the host immune cells are autologous.

51. The method of claim 48, wherein the host immune cells are allogeneic.

52. The method of claim 48, wherein the host immune cells are activated, expanded, or both ex vivo.

53. method of claim 48, wherein the subject has been treated or is being treating with an Fc-containing therapeutic agent.

54. The method of claim 53, wherein the Fc-containing therapeutic agent is a therapeutic antibody or a Fc fusion protein.

55. The method of claim 54, wherein the Fc-containing therapeutic agent is a therapeutic antibody selected from the group consisting of Adalimumab, Ado-Trastuzumab emtansine, Alemtuzumab, Basiliximab, Bevacizumab, Belimumab, Brentuximab, Canakinumab, Cetuximab, Certolizumab, Daclizumab, Denosumab, Dinutuximab, Eculizumab, Efalizumab, Epratuzumab, Gemtuzumab, Golimumab, Ibritumomab, Infliximab, Ipilimumab, Labetuzumab, Muromonab, Natalizumab, Obinutuzumab, Ofatumumab, Omalizumab, Palivizumab, Panitumumab, Pertuzumab, Ramucirumab, Ranibizumab, Rituximab, Toclizumab, Tositumomab, Trastuzumab, Ustekinumab, and Vedolizumab.

56. The method of claim 53, wherein the subject is a human patient suffering from a cancer and the Fc-containing therapeutic agent is for treating the cancer.

57. The method of claim 56, wherein the cancer is selected from the group consisting of carcinoma, lymphoma, sarcoma, blastoma, and leukemia.

58. The method of claim 57, wherein the cancer is selected from the group consisting of a cancer of B-cell origin, breast cancer, gastric cancer, neuroblastoma, osteosarcoma, lung cancer, skin cancer, prostate cancer, colon cancer, renal cell carcinoma, ovarian cancer, rhabdomyosarcoma, leukemia, mesothelioma, pancreatic cancer, head and neck cancer, retinoblastoma, glioma, glioblastoma, and thyroid cancer.

59. The method of claim 58, wherein the cancer of b-cell original is selected from the group consisting of B-lineage acute lymphoblastic leukemia, B-cell chronic lymphocytic leukemia, and B-cell non-Hodgkin's lymphoma.

60. A method for preparing immune cells expressing a chimeric receptor, comprising:
(i) providing a population of immune cells;
(ii) introducing into the immune cells a nucleic acid encoding a chimeric receptor of claim 1; and
(iii) culturing the immune cells under conditions allowing for expression of the chimeric receptor.

61. The method of claim 60, wherein the population of immune cells is derived from peripheral blood mononuclear cells (PBMC).

62. The method of claim 60, wherein the immune cells are natural killer cells, macrophages, neutrophils, eosinophils, T cells, or a combination thereof.

63. The method of claim 60, wherein the immune cells are derived from a human patient.

64. The method of claim 63, wherein the human patient is a cancer patient.

65. The method of claim 60, wherein the nucleic acid is a viral vector.

66. The method of claim 65, wherein the viral vector is a lentiviral vector or a retroviral vector.

67. The method of claim 60, wherein the nucleic acid is an RNA molecule.

68. The method of claim 60, wherein the vector is introduced into the immune cells by lentiviral transduction, retroviral transduction, DNA electroporation, or RNA electroporation.

69. The method of claim 60, further comprising (iv) activating the immune cells expressing the chimeric receptor.

70. The method of claim 69, wherein the immune cells comprise T cells, which are activated in the presence of one or more of anti-CD3 antibody, anti-CD28 antibody, IL-2, and phytohemoagglutinin.

71. The method of claim 70, wherein the immune cells are T cells in which the endogenous T cell receptors are inhibited or eliminated.

72. The method of claim 69, wherein the immune cells comprise natural killer cells, which are activated in the presence of one or more of 4-1BB ligand, anti-4-1BB antibody, IL-15, anti-IL-15 receptor antibody, IL-2, IL-12, IL-21 and K562 cells.


SEQUENCE LISTING (87 sequences)

SEQ ID NO: 1 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 442 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIISFFLALTSTALLFLLFFLTLRFSVVKRGKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 442 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDFWVLVVVGGVLACYSLLVTVAFIIFWVRSKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLIALVTSGALLAVLGITGYFLMNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLLAALLALLAALLALLAALLARSKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 428 aa, Artificial Sequence
  Synthetic Polypeptide
MALPVTALLLPLALLLHAARPQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 435 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCALYLLRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 477 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 391 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGSPAGSPTSTEEGTSESATPESGPGTSTEPSEGSAPGSPAGSPTIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 435 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 477 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRGGHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 437 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDMALIVLGGVAGLLLFIGLGIFFCVRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDMALIVLGGVAGLLLFIGLGIFFCVRRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLCYILDAILFLYGIVLTLLYCRLKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLLLILLGVLAGVLATLAALLARSKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDITLGLLVAGVLVLLVSLGVAIHLCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVSFCLVMVLLFAVDTGLYFSVKTNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVAAILGLGLVLGLLGPLAILLALYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLCYLLDGILFIYGVILTALFLRVKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 441 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVMSVATIVIVDICITGGLLLLVYYWSKNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDGFLFAEIVSIFVLAVGVYFIAGQDKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 437 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDGIIVTDVIATLLLALGVFCFAGHETKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 435 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVIGFRILLLKVAGFNLLMTLRLWKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 438 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIIVAVVIATAVAAIVAAVVALIYCRKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVLFYLAVGIMFLVNTVLWVTIRKEKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIIILVGTAVIAMFFWLLLVIILRTKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 432 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLGWLCLLLLPIPLIVWVKRKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIAIYCIGVFLIACMVVTVILCRMKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 476 aa, Artificial Sequence
  Synthetic Polypeptide
MALPVTALLLPLALLLHAARPQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 623 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 510 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQEPKSCDKTHTCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 403 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQEPKSCDKTHTCPIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 421 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEAFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 406 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 406 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGGGSGGGGSGGGGSIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 421 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGGGSGGGGSGGGGSGGGGSGGGGSGGGGSIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 436 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGGGSGGGGSGGGGSGGGGSGGGGSGGGGSGGGGSGGGGSGGGGSIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 451 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGGGSGGGGSGGGGSGGGGSGGGGSGGGGSGGGGSGGGGSGGGGSGGGGSGGGGSGGGGSIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 451 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGSPAGSPTSTEEGTSESATPESGPGTSTEPSEGSAPGSPAGSPTSTEEGTSTEPSEGSAIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 421 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGSPAGSPTSTEEGTSESATPESGPGTSTEIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 406 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGSPAGSPTSTEEGTIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 433 aa, Artificial Sequence
  Synthetic Polypeptide
MLRLLLALNLFPSIQVTGGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 436 aa, Artificial Sequence
  Synthetic Polypeptide
METDTLLLWVLLLWVPGSTGDGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 431 aa, Artificial Sequence
  Synthetic Polypeptide
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 432 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCCWLTKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 442 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCQRRKYRSNKGESPVEPAEPCRYSCPREEEGSTIPIQEDYRKPEPACSPRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 452 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCQLGLHIWQLRSQCMWPRETQLLLEVPPSTEDARSCQFPEEERGERSAEEKGRLGDLWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 454 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCCVKRRKPRGDVVKVIVSVQRKRQEAEGEATVIEALQAPPDVTTVAVEETIPSFTGRSPNHRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 442 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKKYFFKKEVQQLSVSFSSLQIKALQNAVEKEVQAEDNIYIENSLYATDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 453 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCYKVGFFKRNLKEKMEAGRGVPNGIPAEDSEQLASGQEAGDPGCLKPLHEKDSESGGGKDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 510 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 365 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDPQLCYILDAILFLYGIVLTLLYCRLKIQVRKAAITSYEKSDGVYTGLSTRNQETYETLKHEKPPQKRGRKKLLYIFKQPFMRPVQTTQEEDGCSCRFPEEEEGGCEL

SEQ ID NO: 56 - protein, 366 aa, Artificial Sequence
  Synthetic Polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRLKIQVRKAAITSYEKSDGVYTGLSTRNQETYETLKHEKPPQ

SEQ ID NO: 57 - protein, 192 aa, Artificial Sequence
  Synthetic Polypeptide
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQ

SEQ ID NO: 58 - protein, 69 aa, Artificial Sequence
  Synthetic Polypeptide
TTTPAPRPPTPAPTIASQPLSLRPEACRPAAGGAVHTRGLDFACDIYIWAPLAGTCGVLLLSLVITLYC

SEQ ID NO: 59 - protein, 42 aa, Artificial Sequence
  Synthetic Polypeptide
KRGRKKLLYIFKQPFMRPVQTTQEEDGCSCRFPEEEEGGCEL

SEQ ID NO: 60 - protein, 112 aa, Artificial Sequence
  Synthetic Polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 21 aa, Artificial Sequence
  Synthetic Polypeptide
MALPVTALLLPLALLLHAARP

SEQ ID NO: 62 - DNA, 36 bp, Artificial Sequence
  Synthetic Polynucleotide
cttctgcagggggcttgttgggagtaaaaatgtgtc

SEQ ID NO: 63 - DNA, 36 bp, Artificial Sequence
  Synthetic Polynucleotide
gacacatttttactcccaacaagccccctgcagaag

SEQ ID NO: 64 - DNA, 1311 bp, Artificial Sequence
  Synthetic Polynucleotide
atggccttaccagtgaccgccttgctcctgccgctggccttgctgctccacgccgccaggccgggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccatcactcaaggtttggcagtgtcaaccatctcatcattctttccacctgggtaccaaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 65 - DNA, 576 bp, Artificial Sequence
  Synthetic Polynucleotide
ggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccatcactcaaggtttggcagtgtcaaccatctcatcattctttccacctgggtaccaa

SEQ ID NO: 66 - DNA, 207 bp, Artificial Sequence
  Synthetic Polynucleotide
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgc

SEQ ID NO: 67 - DNA, 126 bp, Artificial Sequence
  Synthetic Polynucleotide
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 68 - DNA, 339 bp, Artificial Sequence
  Synthetic Polynucleotide
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 69 - DNA, 63 bp, Artificial Sequence
  Synthetic Polynucleotide
atggccttaccagtgaccgccttgctcctgccgctggccttgctgctccacgccgccaggccg

SEQ ID NO: 70 - protein, 192 aa, Artificial Sequence
  Synthetic Polypeptide
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQ

SEQ ID NO: 71 - DNA, 1311 bp, Artificial Sequence
  Synthetic Polynucleotide
atggccttaccagtgaccgccttgctcctgccgctggccttgctgctccacgccgccaggccgggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 72 - DNA, 576 bp, Artificial Sequence
  Synthetic Polynucleotide
ggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaa

SEQ ID NO: 73 - DNA, 36 bp, Artificial Sequence; identical to SEQ ID NO: 62
  Synthetic Polynucleotide

SEQ ID NO: 74 - DNA, 36 bp, Artificial Sequence; identical to SEQ ID NO: 63
  Synthetic Polynucleotide

SEQ ID NO: 75 - protein, 254 aa, Artificial Sequence
  Synthetic Polypeptide
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 76 - protein, 5 aa, Artificial Sequence
  Synthetic Polypeptide
GGGGS

SEQ ID NO: 77 - protein, 15 aa, Artificial Sequence
  Synthetic Polypeptide
GGGGSGGGGSGGGGS

SEQ ID NO: 78 - protein, 30 aa, Artificial Sequence
  Synthetic Polypeptide
GGGGSGGGGSGGGGSGGGGSGGGGSGGGGS

SEQ ID NO: 79 - protein, 45 aa, Artificial Sequence
  Synthetic Polypeptide
GGGGSGGGGSGGGGSGGGGSGGGGSGGGGSGGGGSGGGGSGGGGS

SEQ ID NO: 80 - protein, 60 aa, Artificial Sequence
  Synthetic Polypeptide
GGGGSGGGGSGGGGSGGGGSGGGGSGGGGSGGGGSGGGGSGGGGSGGGGSGGGGSGGGGS

SEQ ID NO: 81 - protein, 75 aa, Artificial Sequence
  Synthetic Polypeptide
GGGGSGGGGSGGGGSGGGGSGGGGSGGGGSGGGGSGGGGSGGGGSGGGGSGGGGSGGGGSGGGGSGGGGSGGGGS

SEQ ID NO: 82 - protein, 150 aa, Artificial Sequence
  Synthetic Polypeptide
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 83 - protein, 225 aa, Artificial Sequence
  Synthetic Polypeptide
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 84 - protein, 300 aa, Artificial Sequence
  Synthetic Polypeptide
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 85 - protein, 20 aa, Artificial Sequence
  Synthetic Polypeptide
ETQRCTWHMGELVWCEREHN

SEQ ID NO: 86 - protein, 20 aa, Artificial Sequence
  Synthetic Polypeptide
KEASCSYWLGELVWCVAGVE

SEQ ID NO: 87 - protein, 13 aa, Artificial Sequence
  Synthetic Polypeptide
DCAWHLGELVWCT